(19) United States
(12) Patent Application Publication
(10) Pub. No.: US 2023/0101980 A1
(43) Pub. Date: Mar. 30, 2023

(54) METHOD, SYSTEM AND APPARATUS FOR SUBSTANCE IDENTIFICATION

(71) Applicant: Trillitye PAULLIN, Meridian, ID (US)

(72) Inventor: Trillitye PAULLIN, Meridian, ID (US)

(73) Assignee: Trillitye PAULLIN, Meridian, ID (US)

(21) Appl. No.: 17/611,894

(22) PCT Filed: May 18, 2020
(86) PCT No.: PCT/US20/33489

§ 371 Date: Nov. 16, 2021

Related U.S. Application Data
(60) Provisional application No. 62/849,032, filed on May 16, 2019.

(51) Int. Cl. G01N 33/53; G01N 33/543; G01N 33/68

(52) U.S. Cl. CPC G01N 33/5308; G01N 33/54366; G01N 33/6848

(57) ABSTRACT

This disclosure generally relates to embodiments for detecting presence of one or more allergens in mammalian milk. An exemplary embodiment relates to a method to detect presence of one or more allergen molecules in a composition of mammalian milk, the method includes the steps of: providing a substrate having a plurality of detection sites thereon, each of the plurality of detection sites configured to detect presence of one or more allergen molecules; exposing the plurality of detection sites to a quantity of mammalian milk; detecting presence of a first allergen molecule at a first of the plurality of detection sites by detecting a fragment of DNA, RNA, or amino acids corresponding to the first allergen molecule; wherein the detected fragment excludes naturally occurring molecules present in the composition of mammalian milk.

The instant disclosure claims priority to the filing date of Provisional Application No. 2/849,032, filed May 16, 2019, the specification of which is incorporated herein in its entirety.

### BACKGROUND

### Field

The disclosure generally relates to method, system and apparatus for substance identification. In certain embodiments, the disclosure provides methods to identify presence of at least partially digested enzymes in mammalian liquid to indicate presence of one or more allergens.

### Description of the Related Art

Various testing substrates, some commonly known as test strips, are often used in many fields, including the chemical, medical, and veterinary arts, as well as in various industry sectors such as the food, medical, or cosmetic industries. These strips, as they are known, enable quick and hassle-free testing because they can be used in situ and their application often involves only exposing the strip to the substance to be tested. Accordingly, they are inexpensive, accessible, portable, and easy to use. These features are among the assorted attributes that make them useful and popular.

There are limitations which impact the utility of these test strips. For example, test strips may not provide identification of a desirable substance, a sufficient number of substances, or the identification may not be as accurate as necessary. A combination of the ability to identify and provide error-free results has not been achieved in the products currently available—even though a great need exists for an inexpensive, accessible, and easy manner of testing and identifying substances.

### SUMMARY

According to embodiments of the disclosure, systems, apparatuses, kits, and methods are provided which allow testing of one or more substances when the substrate according to the present disclosure is brought into contact therewith. The present disclosure contemplates use of interfaces including software applications with the systems, apparatuses, kits, and methods for further benefit.

An exemplary embodiment of the disclosure provides a substrate having thereon a plurality of active sites (interchangeably, detection site). Each active site comprise site may comprise one or more detection agents. Each detection agent may comprise one or more probes with an active binding site. The binding site may comprise one or more components configured to selectively bind to a target molecule. The target molecule may comprise an enzyme, a partially digested enzyme or a substance whose presence is detectable.

In another exemplary embodiment, each detection site is in communication with a circuitry such that upon binding of a target molecule at a detection site, an electronic signal is generated to identify detection of the target molecule.

In certain embodiments, a substrate may comprise a plurality of active site. Each detection site may be configured to detect presence of a target molecule. The target molecule may be a protein, a partially digested protein, an epitope or a molecular fraction of a larger molecule that was subjected to human digestion. In an implementation, the detection sites may be configured to exclusively detect the presence of the target molecule. In another implementation, the detection sites may be configured to detect presence of the target molecule without detecting presence of a host protein, a partially digested host protein or other molecular structure commonly present in the host.

In still another exemplary embodiment, the plurality of detection sites may be configured to detect the presence of fragments of DNA, RNA, or amino acids which are specific to the target molecule. In another embodiment, the plurality of detection sites may be configured to detect the presence of fragments of DNA, RNA, or amino acids which may be specific to the target molecule and do not occur naturally nor are expectedly present in the host. In one embodiment, each of the plurality of detection sites may be configured to detect the presence of the same fragment of a target molecule. In some embodiments, each of the plurality of target sites may be configured to detect the presence of a different fragment of the same target molecule. The different fragments may be specifically selected from a portions of the target molecule to produce a signature specific and unique to the target molecule.

### BRIEF DESCRIPTION OF DRAWINGS

The following drawings are exemplary and illustrative of the disclosed embodiments, in which like elements are numbered similarly and where:

FIG. 1 Illustrates a substance identifying substrate according to an embodiment of the disclosure;

FIG. 2 Illustrates a side view of the embodiment as illustrated in FIG. 1;

FIG. 3 Illustrates a substance identifying substrate according to another embodiment of the disclosure;

FIG. 4 Illustrates a substance identifying substrate according to yet another embodiment of the disclosure;

FIG. 5 is a schematic illustration of a detection substrate 600 according to one embodiment of the disclosure;

FIG. 6 schematically illustrates a circuitry for indicating presence of a target molecule according to one embodiment of the disclosure;

FIG. 7 represents Table 2 which is an exemplary listing of sequences relating to different allergens that may be used to identify fragments of interest;

FIG. 8 shows exemplary fragments of interest for αₛ₁-casein protein which can be found in bovine milk;

FIG. 9 shows exemplary fragments of interest for αₛ₂-casein protein which can be found in bovine milk;

FIG. 10 shows exemplary fragments of interest for β-casein protein which can be found in bovine milk;

FIG. 11 shows exemplary fragments of interest for β-casein protein which can be found in bovine milk;

FIG. 12 shows exemplary fragments of interest for Pancreatic trypsin inhibitor which can be found in bovine milk;

FIG. 13 shows exemplary fragments of interest for β-lactoglobulin which can be found in bovine milk;

FIG. 14 shows exemplary fragments of interest for Lactoferrin which can be found in bovine milk;

FIG. 15 shows exemplary fragments of interest for β2-microglobulin which can be found in bovine milk;

FIG. 16 shows exemplary fragments of interest for serum albumin which can be found in chicken;

FIG. 17 shows exemplary fragments of interest for ovalbumin protein which can be found in chicken;

FIG. 18 shows exemplary fragments of interest for gallus protein which can be found in chicken;

FIG. 19 shows exemplary fragments of interest for Ovomuvoid protein which can be found in chicken;

FIG. 20 shows exemplary fragments of interest for Ovoglobulin G2/G3 protein which can be found in chicken;

FIG. 21 shows exemplary fragments of interest for Ovomucin protein which can be found in chicken;

FIG. 22 shows exemplary fragments of interest for lysozyme protein which can be found in chicken;

FIG. 23 shows exemplary fragments of interest for α-parvalbumin proteins which can be found in salmon and pike fish;

FIG. 24 shows exemplary fragments of interest for α-parvalbumin protein which can be found in pike fish;

FIG. 25 shows exemplary fragments of interest for α-enolase protein which can be found in tuna;

FIG. 26 shows exemplary fragments of interest for β-enolase protein which can be found in salmon;

FIG. 27 shows exemplary fragments of interest for β-enolase protein which can be found in carp;

FIG. 28 shows exemplary fragments of interest for [symbol] -enolase protein which can be found in salmon;

FIG. 29 shows exemplary fragments of interest for [symbol] -enolase protein which can be found in salmon;

FIG. 30 shows exemplary fragments of interest for Tropomyosin protein which can be found in crustacean;

FIG. 31 shows exemplary fragments of interest for Tropomyosin protein which can be found in crustacean (crab);

FIG. 32 shows exemplary fragments of interest for Arginine kinase protein which can be found in crustacean (crab);

FIG. 33 shows exemplary fragments of interest for Vicilins protein which can be found in tree nuts (pecan, walnut);

FIG. 34 shows exemplary fragments of interest for Vicilins protein which can be found in tree nuts (cashew, pistachio);

FIG. 35 shows exemplary fragments of interest for Vicilins protein which can be found in tree nuts (macadamia);

FIG. 36 shows exemplary fragments of interest for Vicilins protein which can be found in tree nuts (almond);

FIG. 37 shows exemplary fragments of interest for Vicilins protein which can be found in tree nuts (coconut);

FIG. 38, shows exemplary fragments of interest for Globulin protein which can be found in tree nuts (pecan, walnut, chestnut);

FIG. 39 shows exemplary fragments of interest for Globulin protein which can be found in tree nuts (cashew, pistachio), the exemplary fragments of FIG. 39 do not show human comparatives;

FIG. 40 shows exemplary fragments of interest for Globulin protein which can be found in tree nuts (almond);

FIG. 41 shows exemplary fragments of interest for Globulin protein which can be found in tree nuts (cashew and pistachio);

FIG. 42 shows exemplary fragments of interest for Profilin protein which can be found in tree nuts (hazelnut);

FIG. 43 shows exemplary fragments of interest for 2S Albumin protein which can be found in tree nuts (hazelnut);

FIG. 44 shows exemplary fragments of interest for 2S Albumin protein which can be found in tree nuts (cashew);

FIG. 45 shows exemplary fragments of interest for LTPs which can be found in tree nuts (hazelnut);

FIG. 46 shows exemplary fragments of interest for legumins protein which can be found in legumes (pea, chickpea);

FIG. 47 shows exemplary fragments of interest for Globulins protein which can be found in legumes (soybean, pea, chickpea and peanut);

FIG. 48 shows exemplary fragments of interest for Globulin-Ara h 1 which can be found in legumes (peanut);

FIG. 49 shows exemplary fragments of interest for 2S Albumin (Prolamin-Ara h 2) which can be found in legumes (peanut);

FIG. 50 shows exemplary fragments of interest for Glycinin-Ara h 3 protein which can be found in legumes (peanuts);

FIG. 51 shows exemplary fragments of interest for Prolamins protein which can be found in legumes (soybean);

FIG. 53 shows exemplary fragments of interest for Glutenin which can be found in cereals/grains (wheat, rye);

FIG. 54 shows exemplary fragments of interest for Avenin 3 (prolamin) which can be found in cereals/grains (oat);

FIG. 55 shows exemplary fragments of interest for Avenin 3 (prolamin) which can be found in cereals/grains (corn);

FIG. 56 shows exemplary fragments of interest for [symbol] -Prolamin which can be found in cereals/grains (wheat, rye, barley);

FIG. 57 shows fragments of interest for Gliadin (prolamin) which can be found in cereals/grains (wheat);

FIG. 58 shows fragments of interest for Proline aminopeptidase 1/Leucyl aminopeptidase which can be found in cereals/grains (rice); and

FIG. 59 shows fragments of interest for Profilin3 protein which can be found in found in cereals/grains (corn).

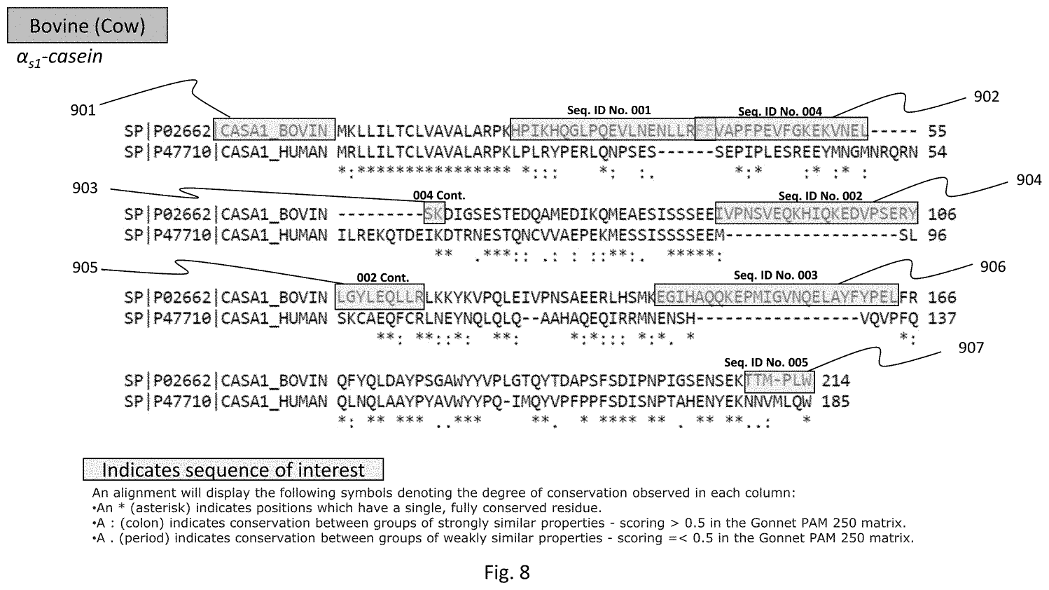

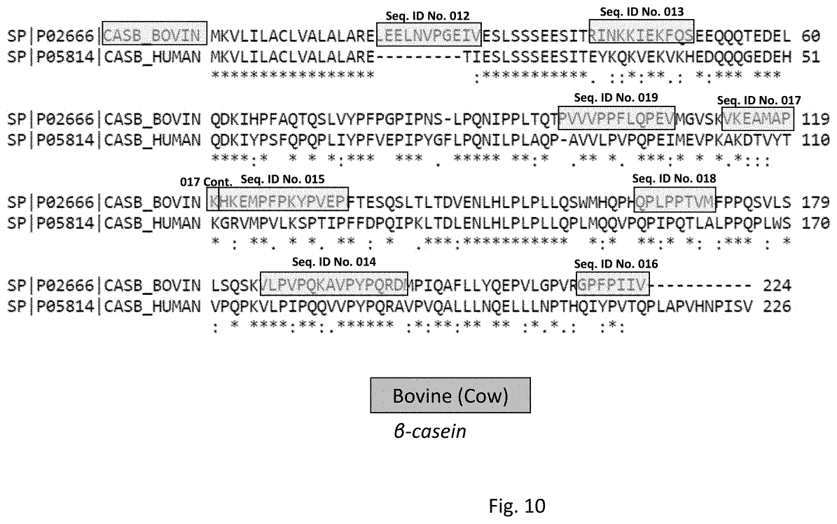

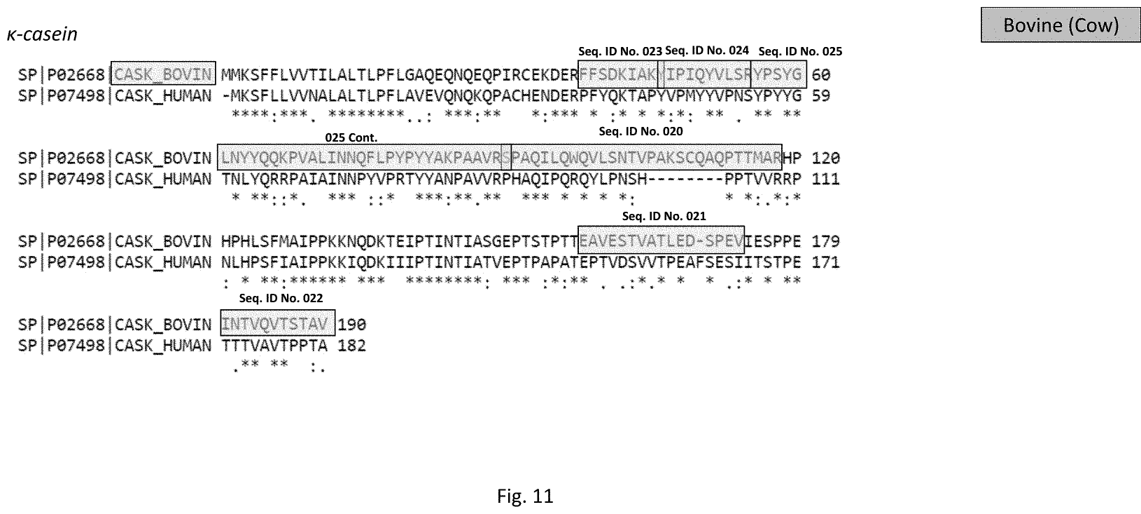

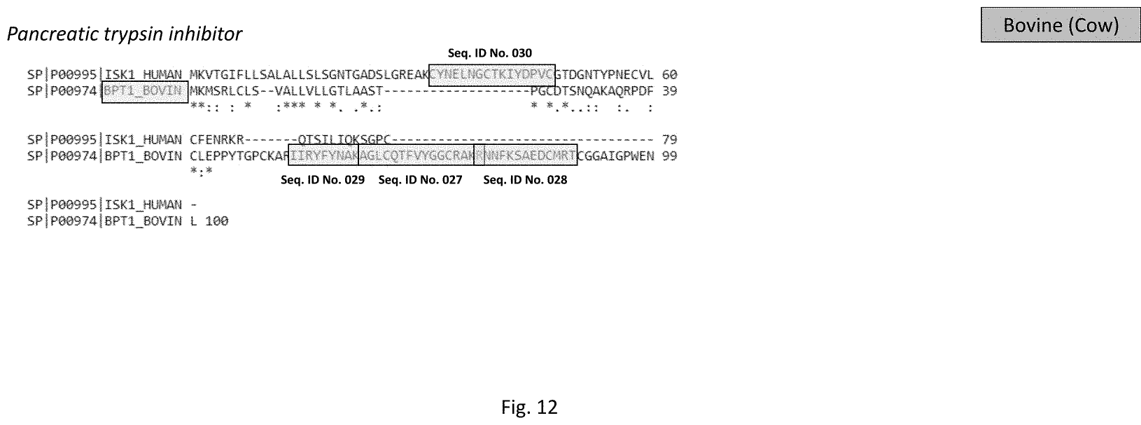

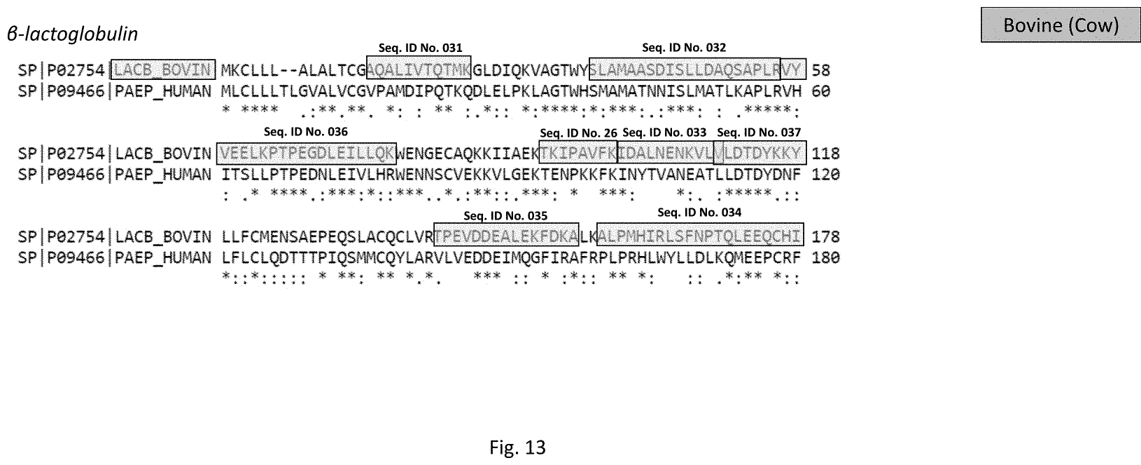

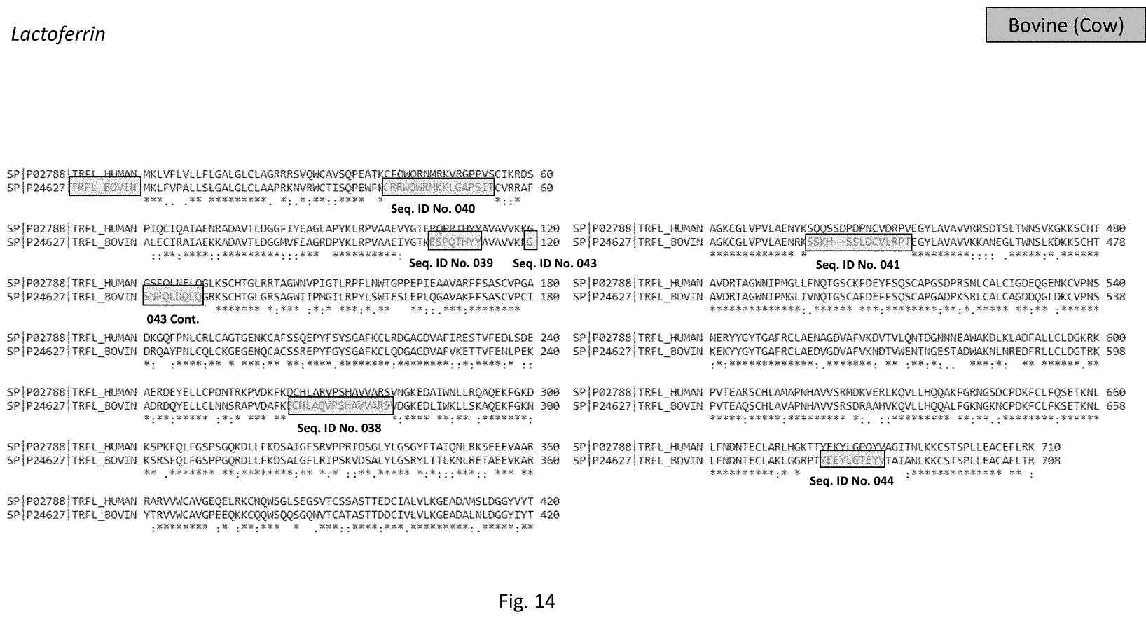

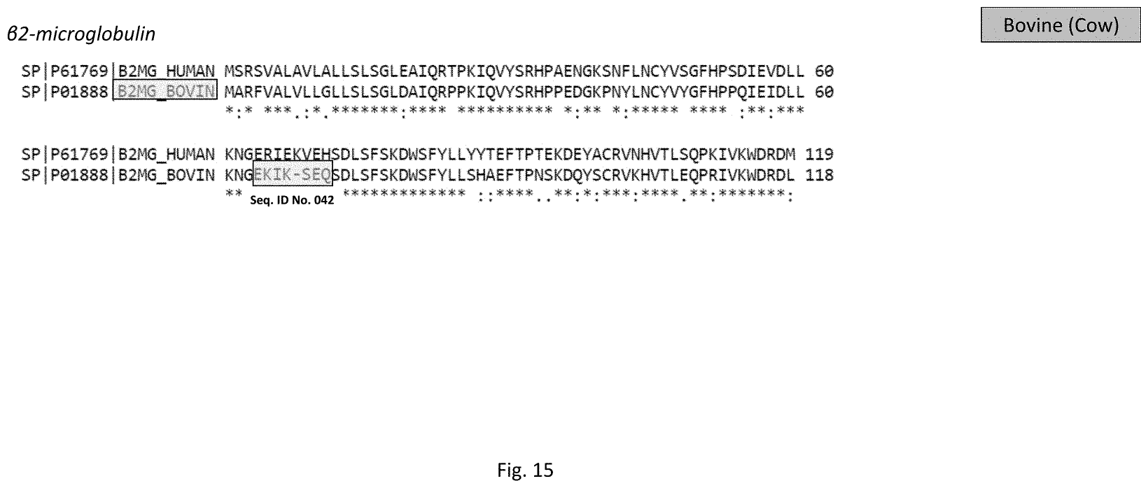

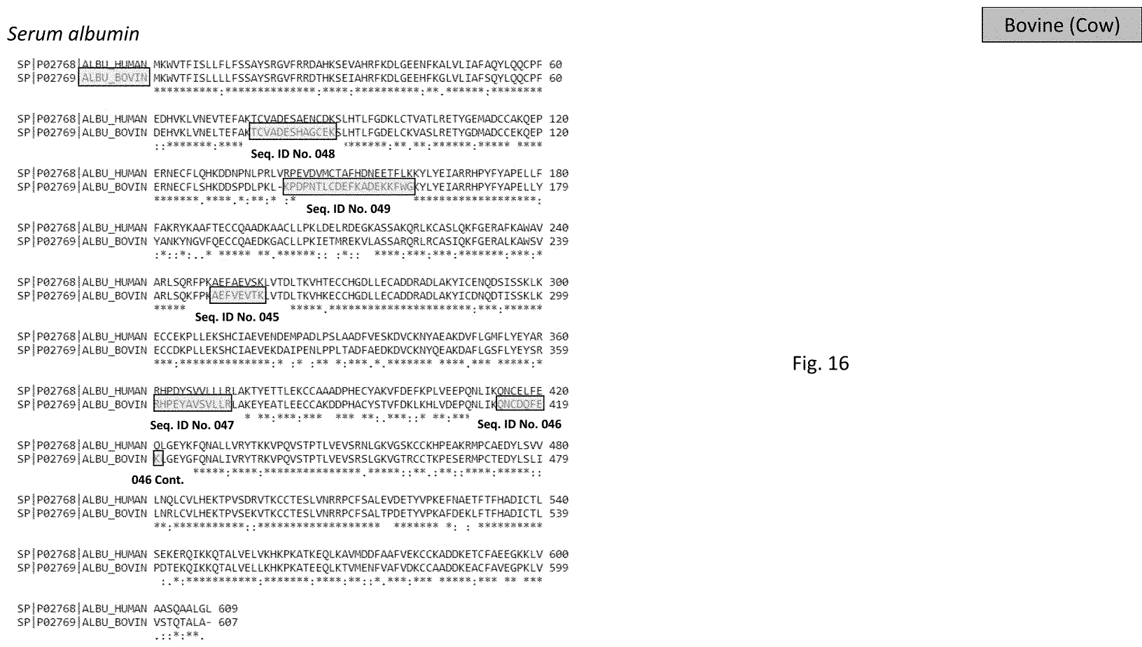

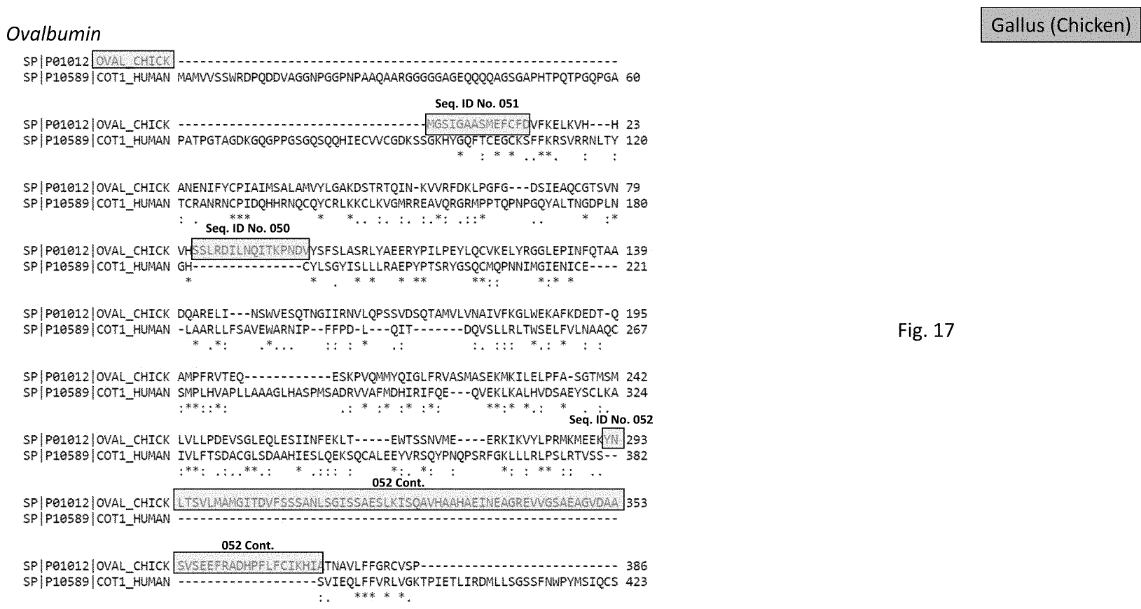

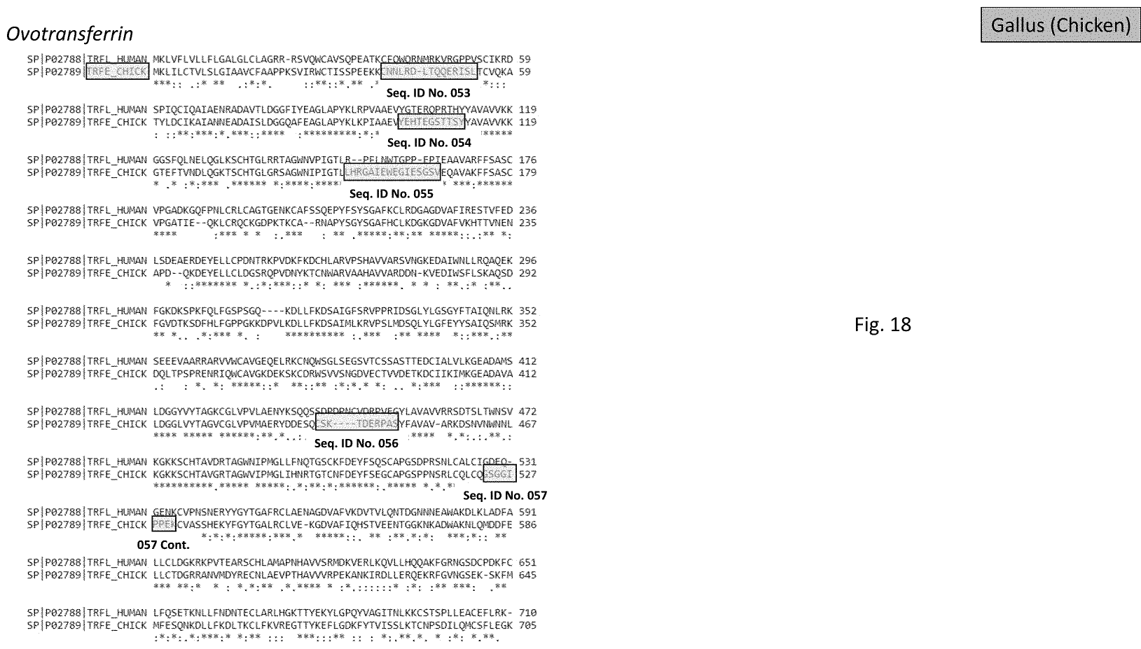

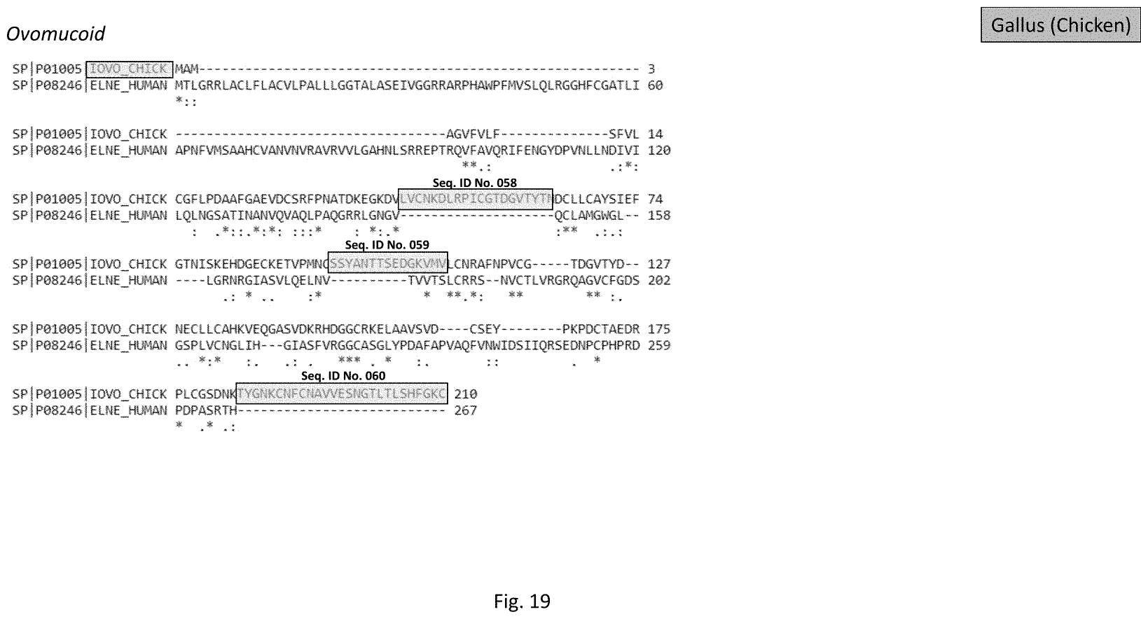

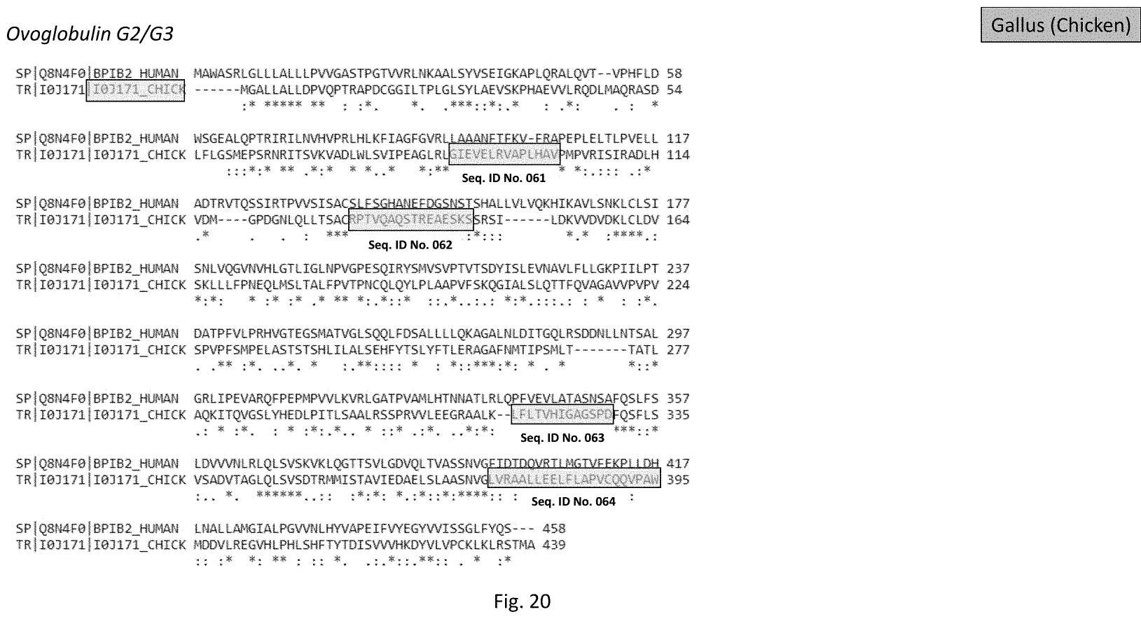

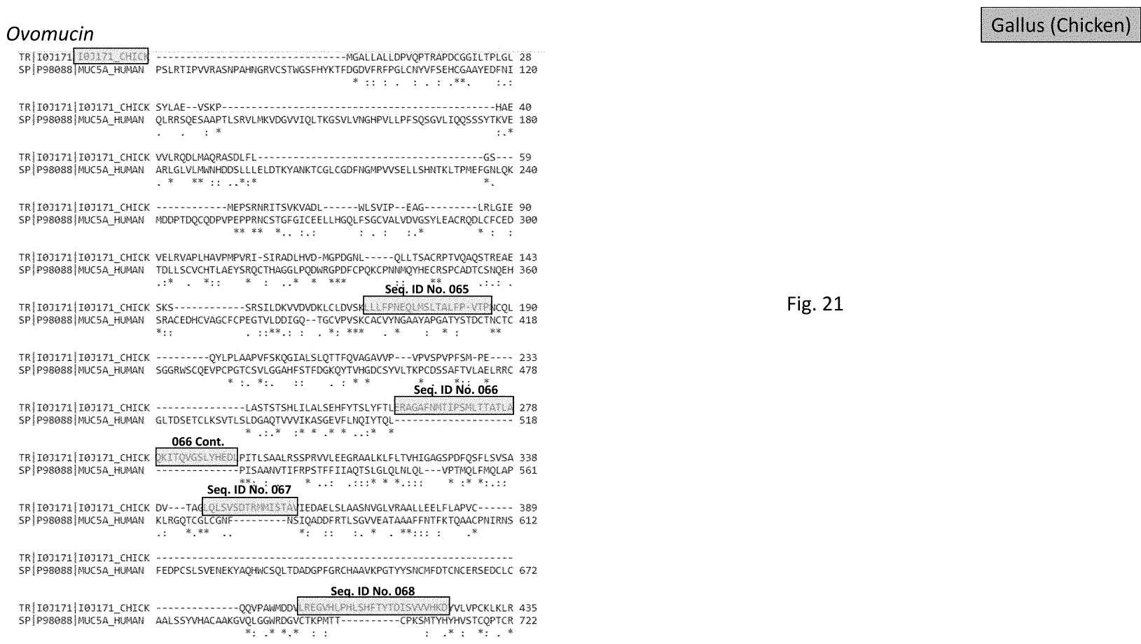

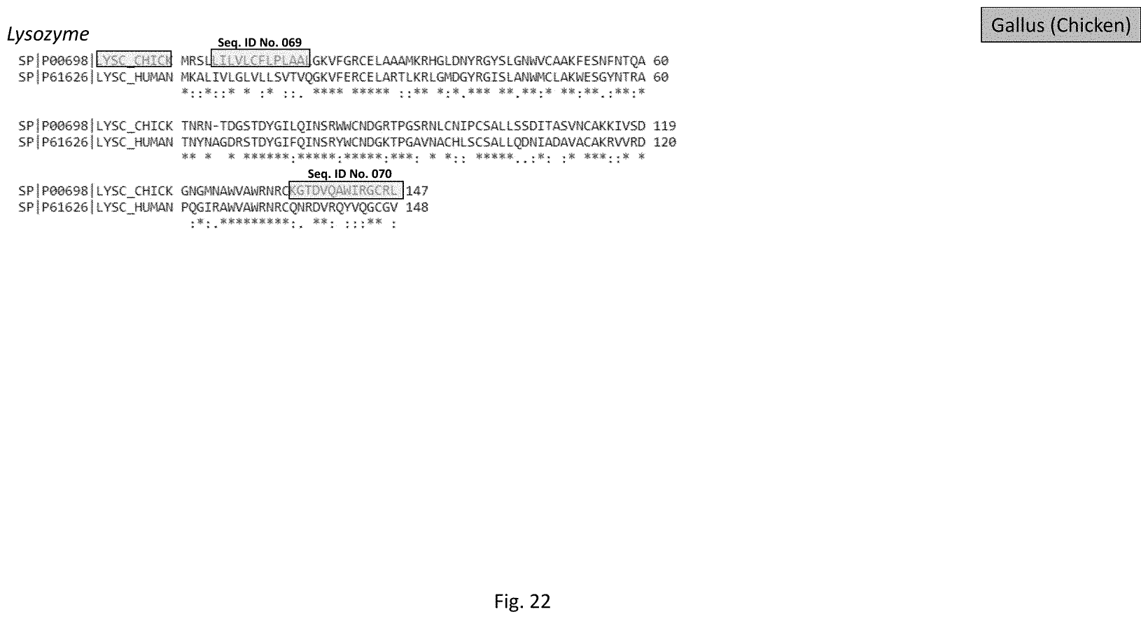

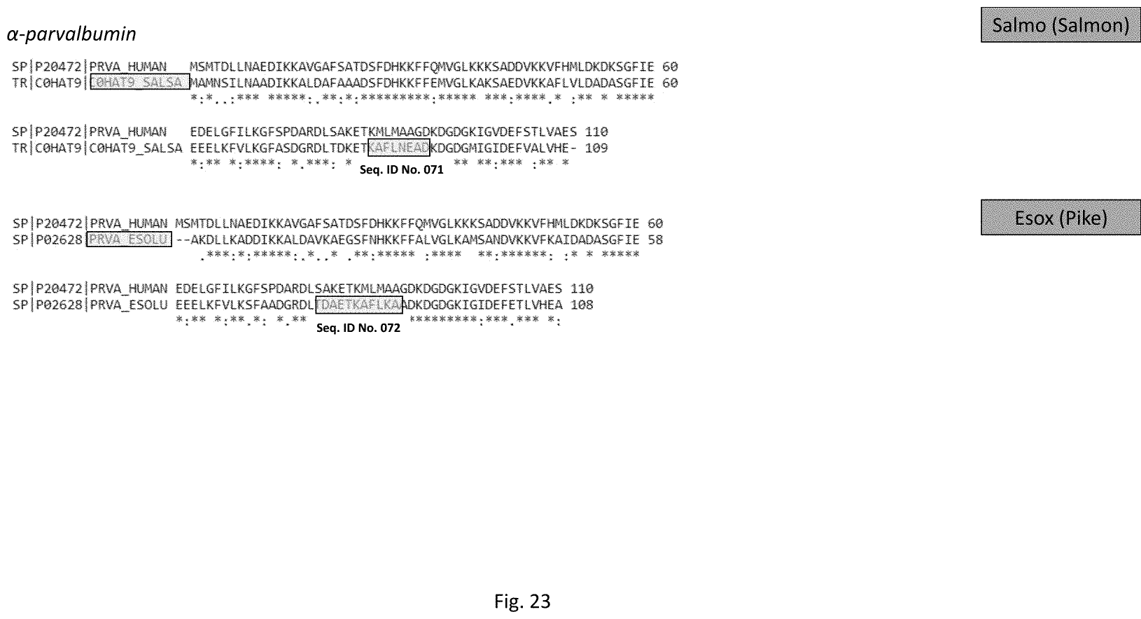

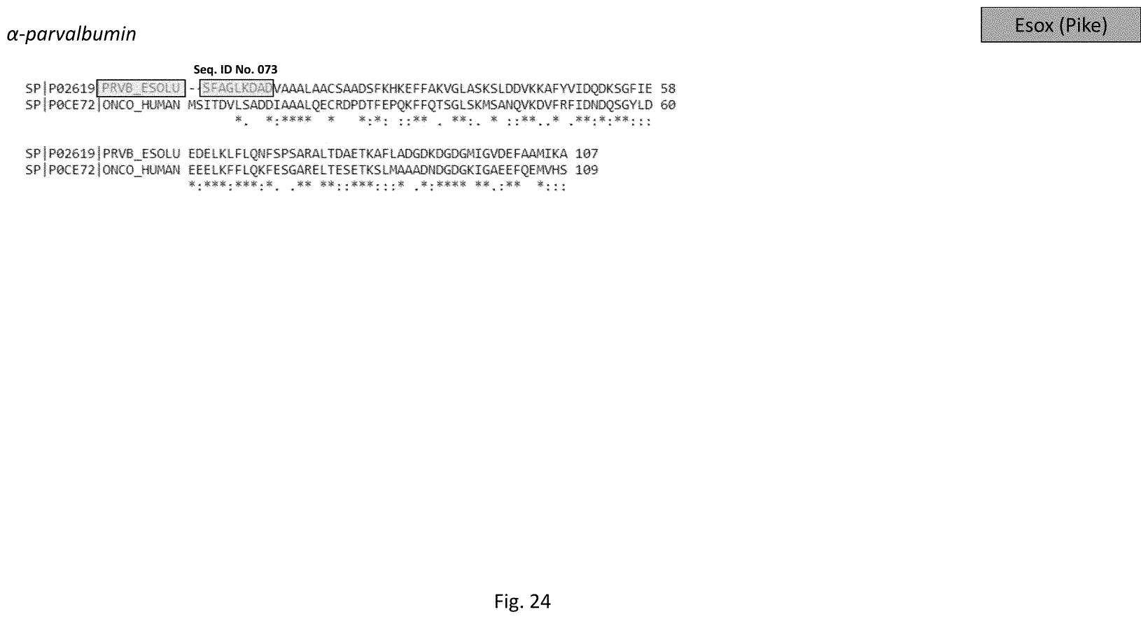

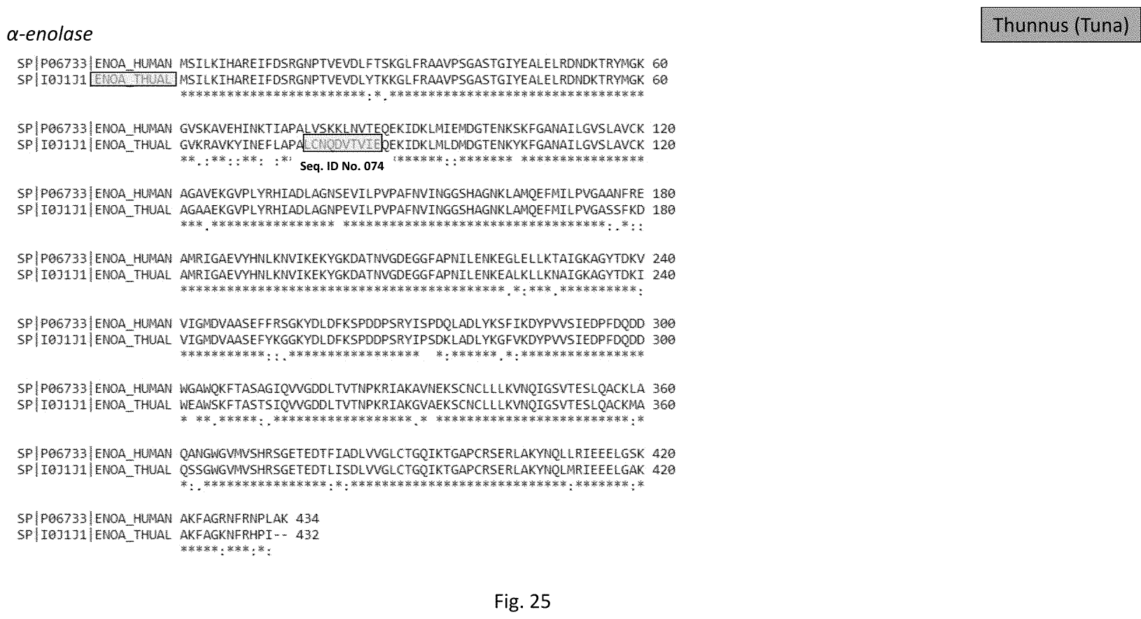

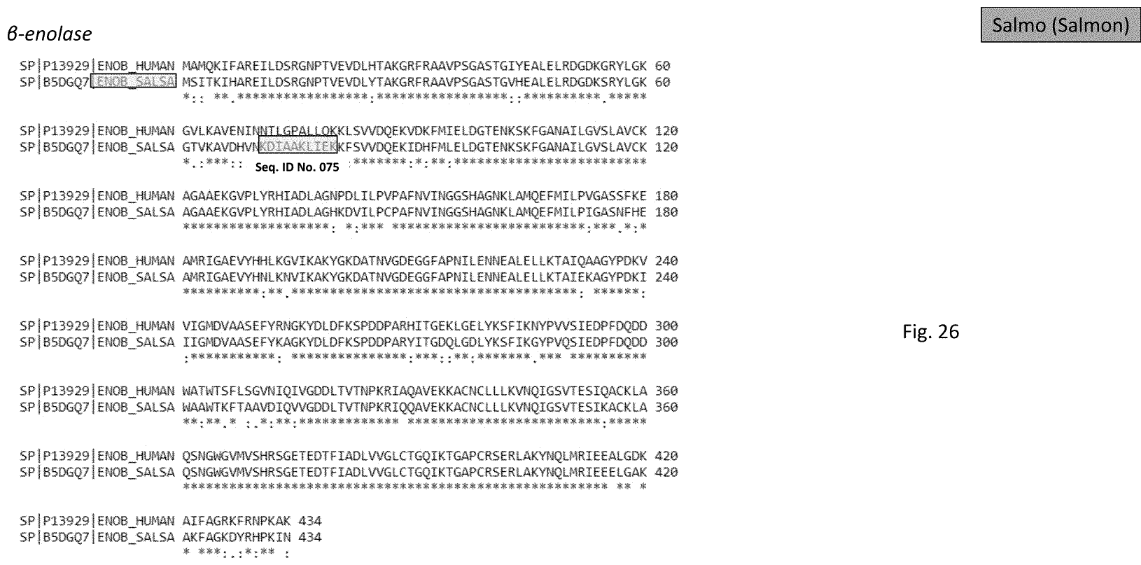

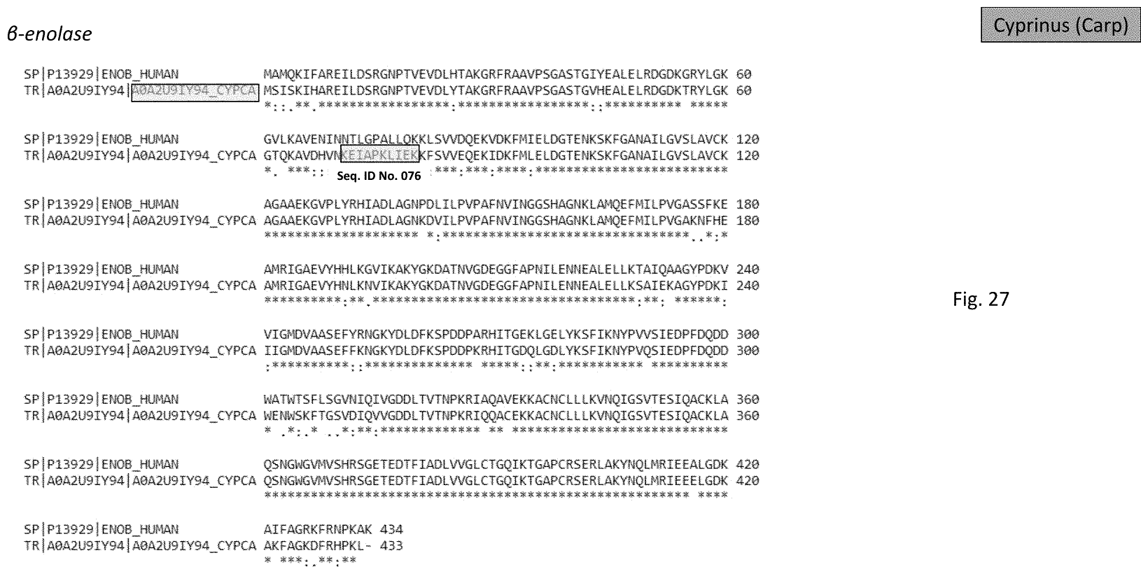

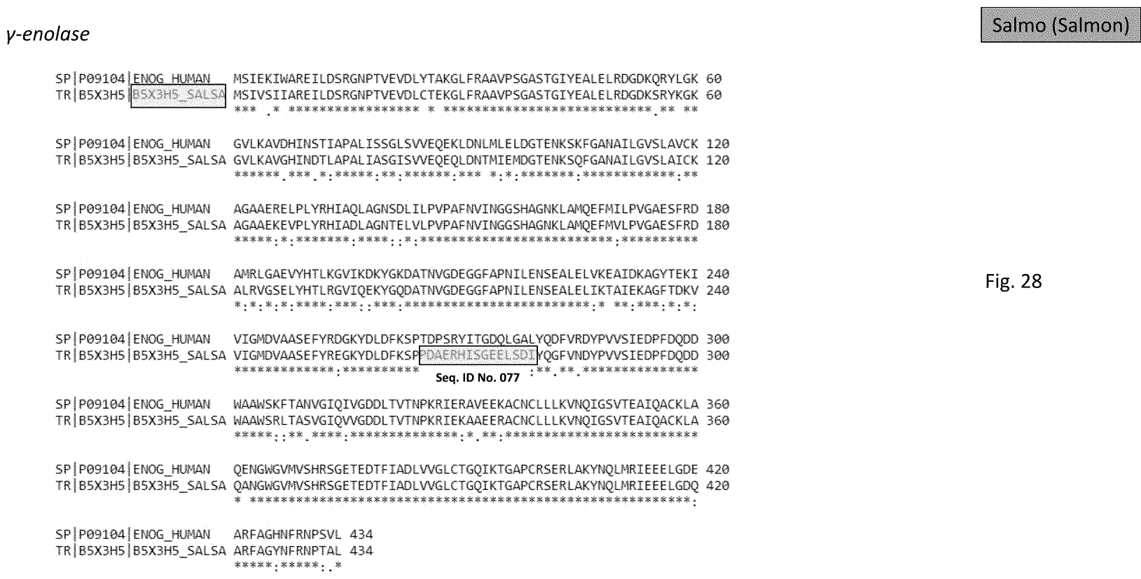

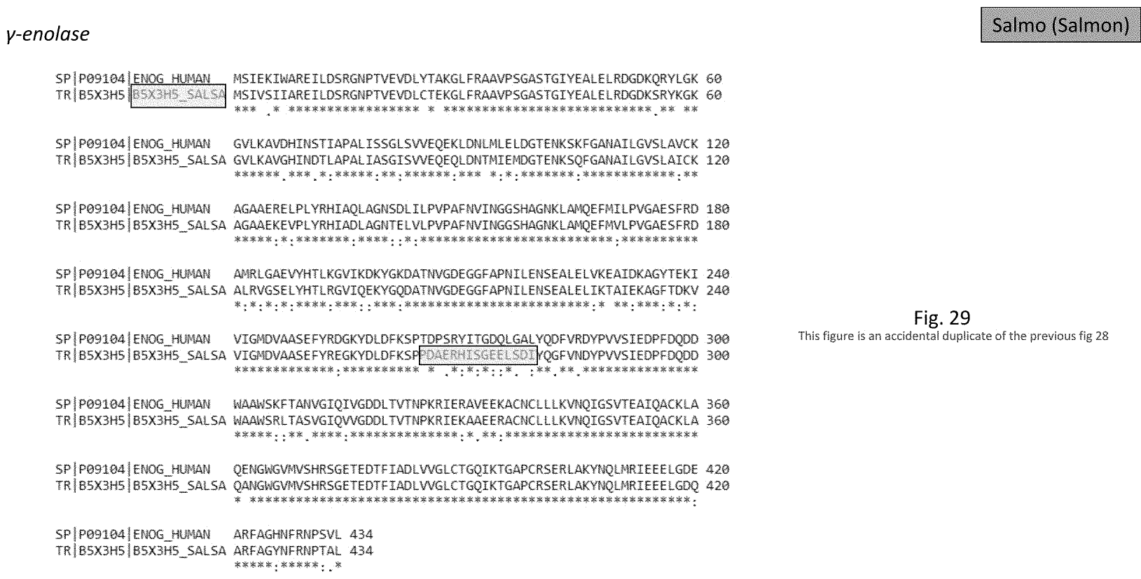

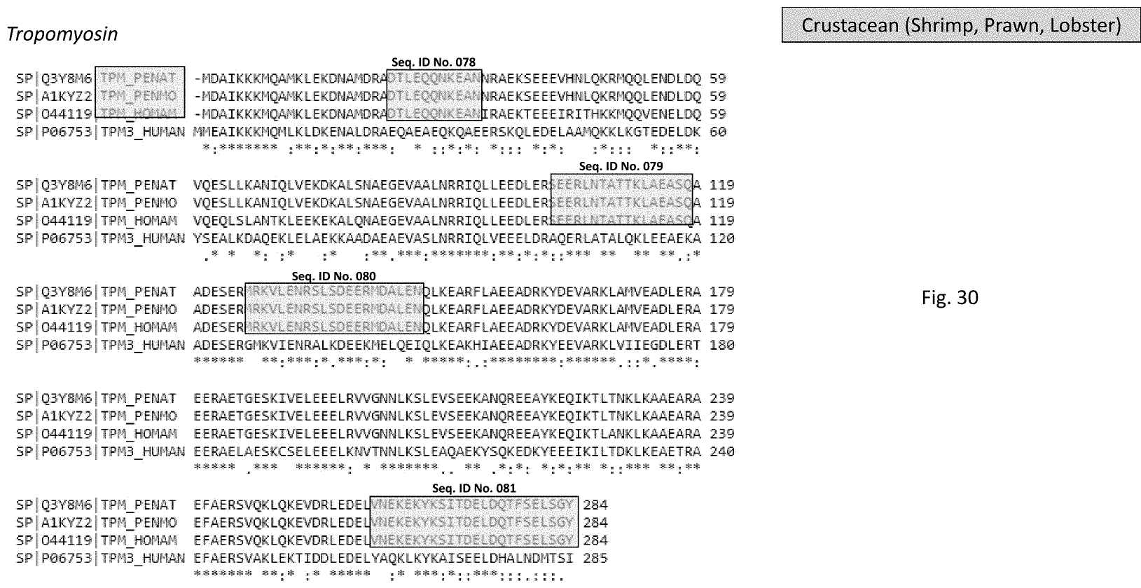

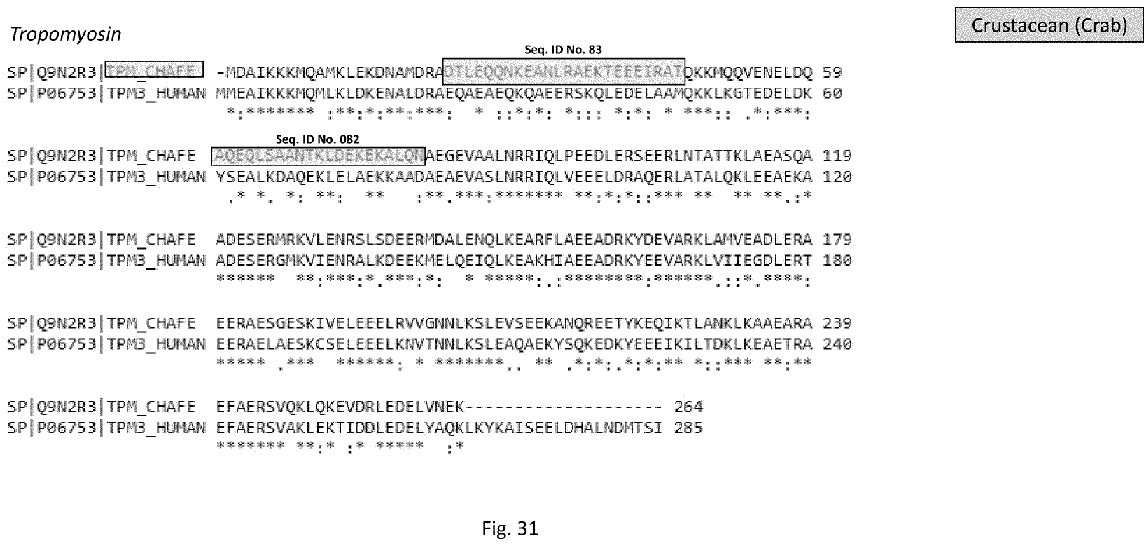

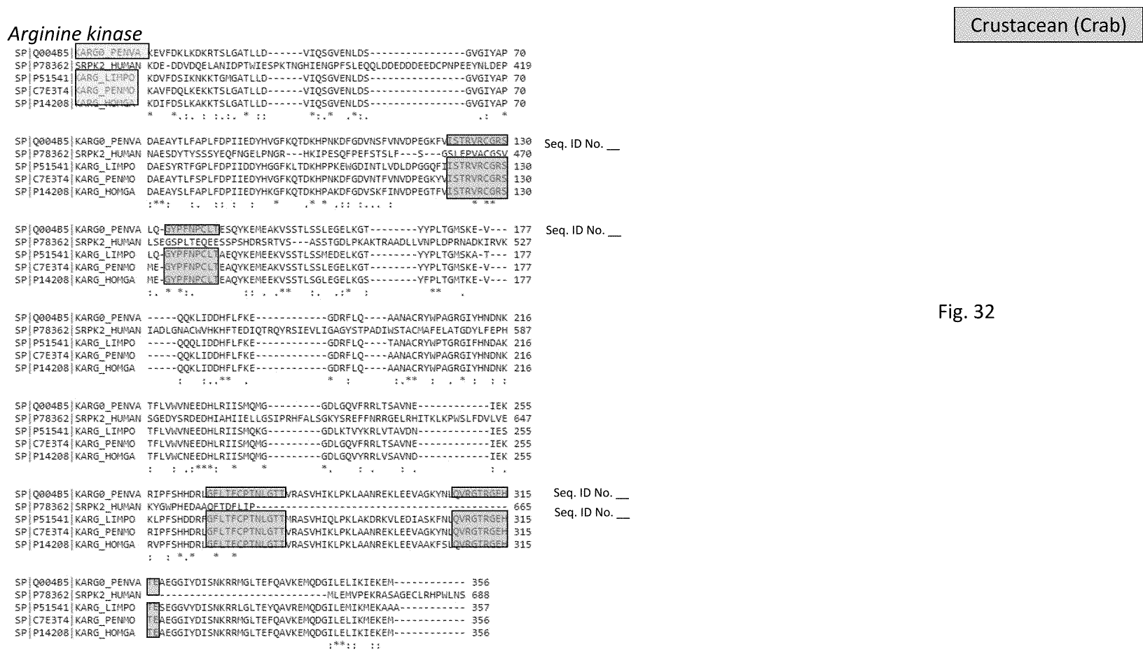

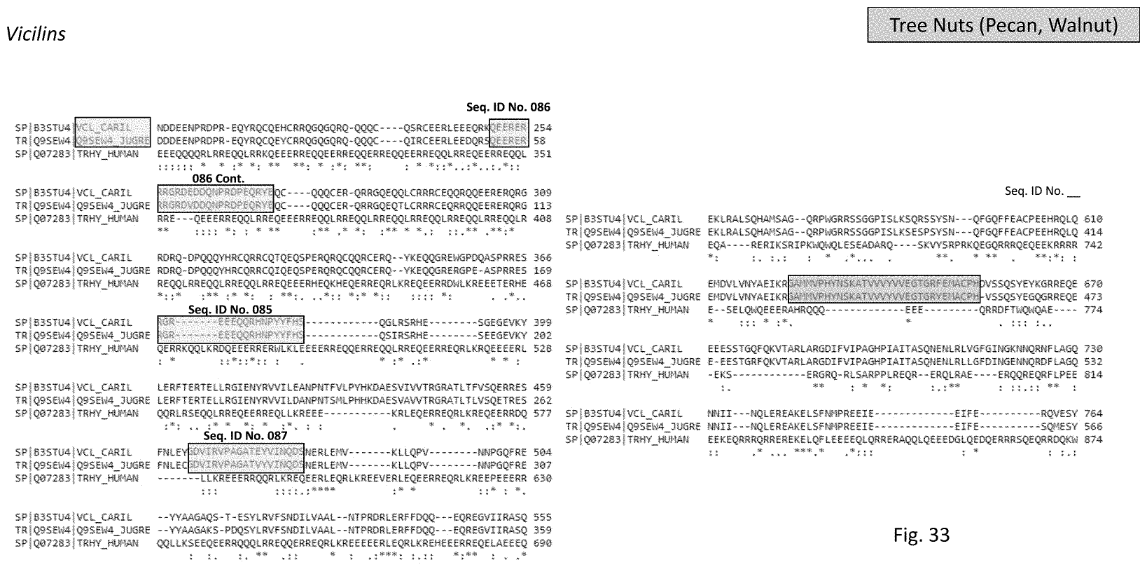

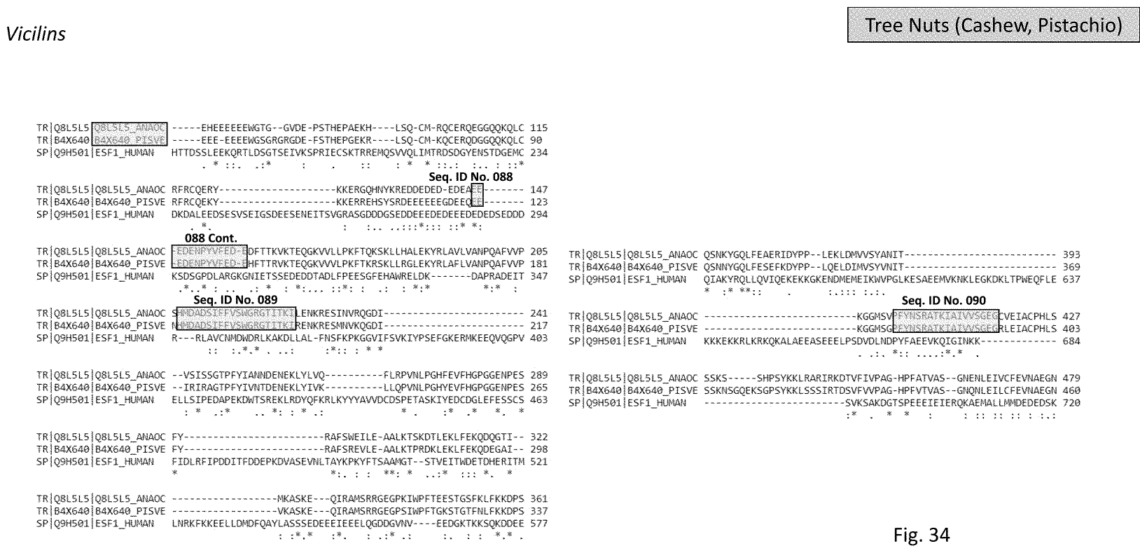

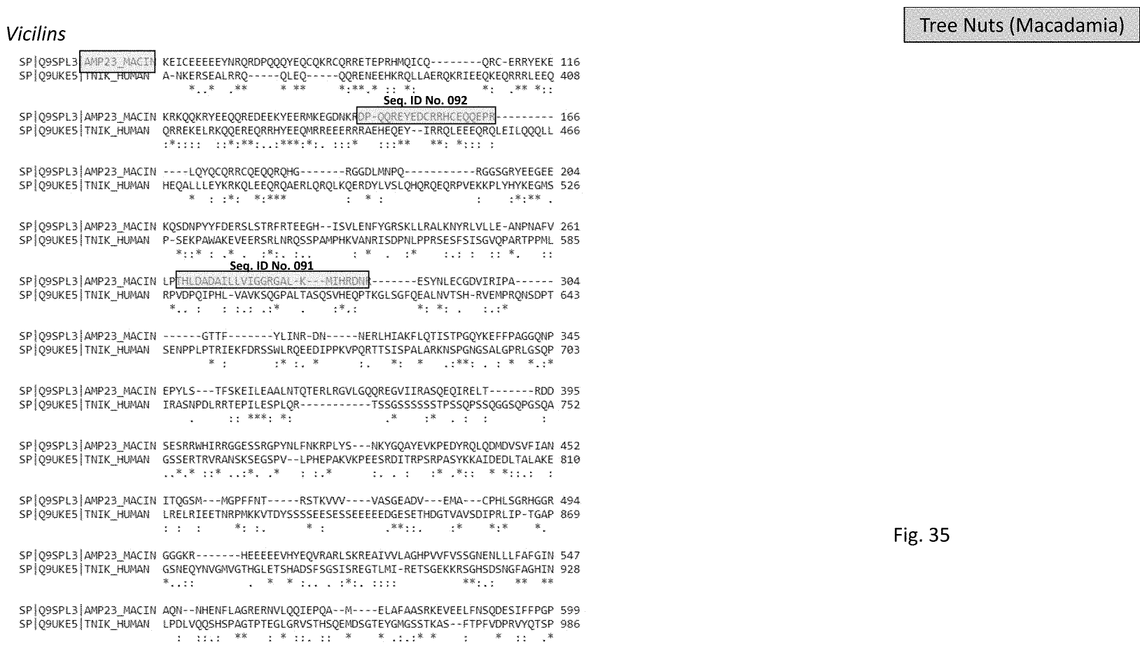

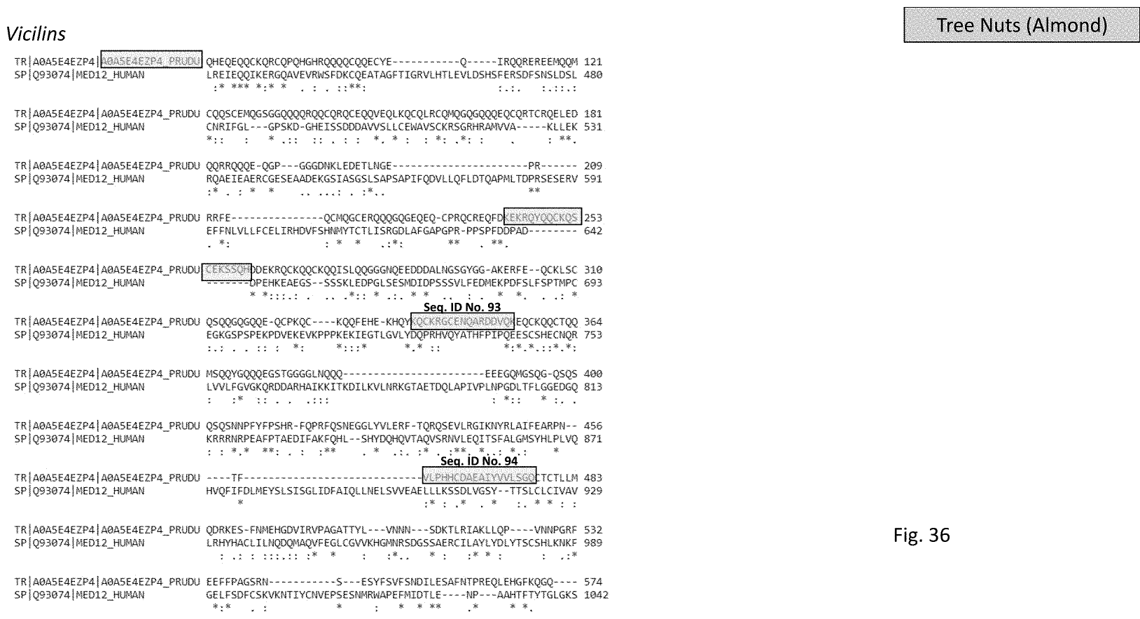

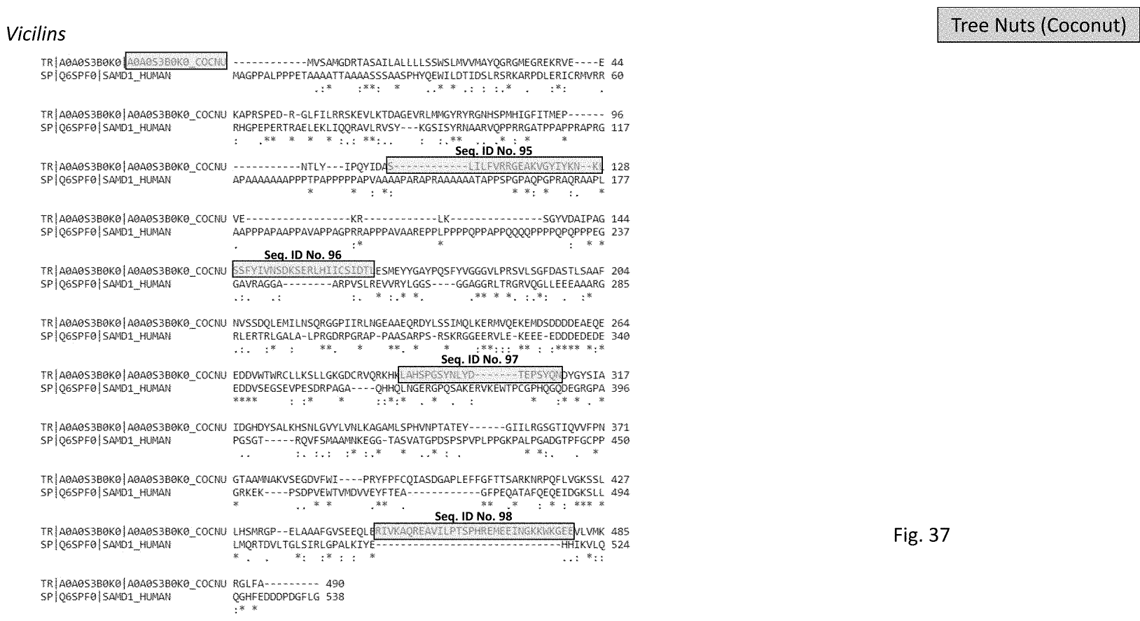

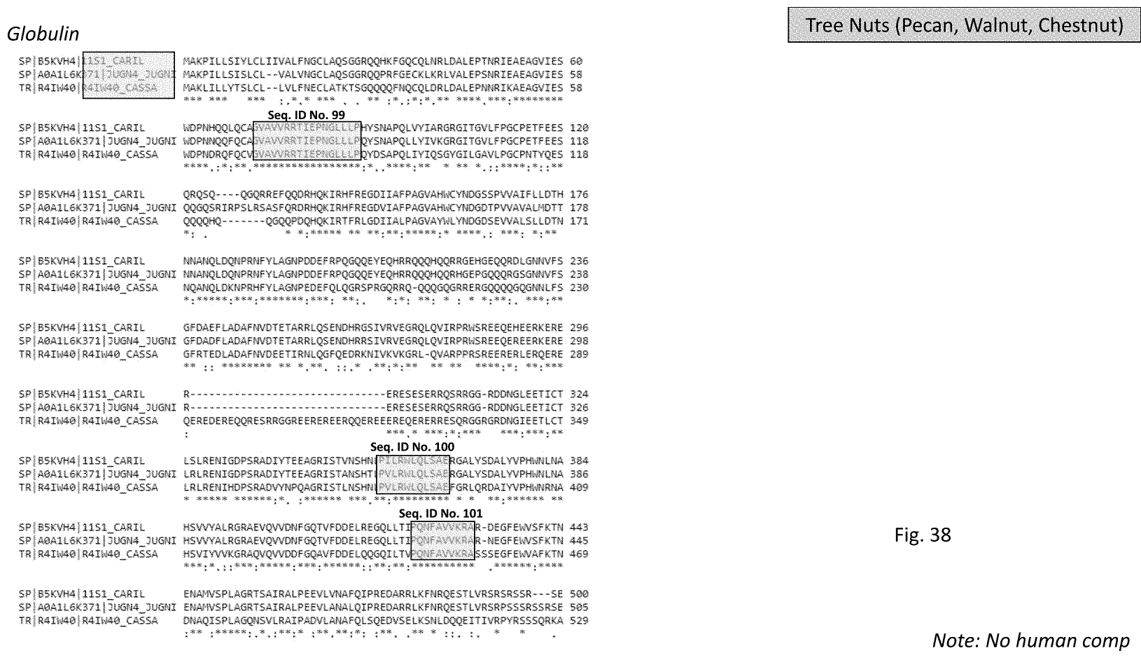

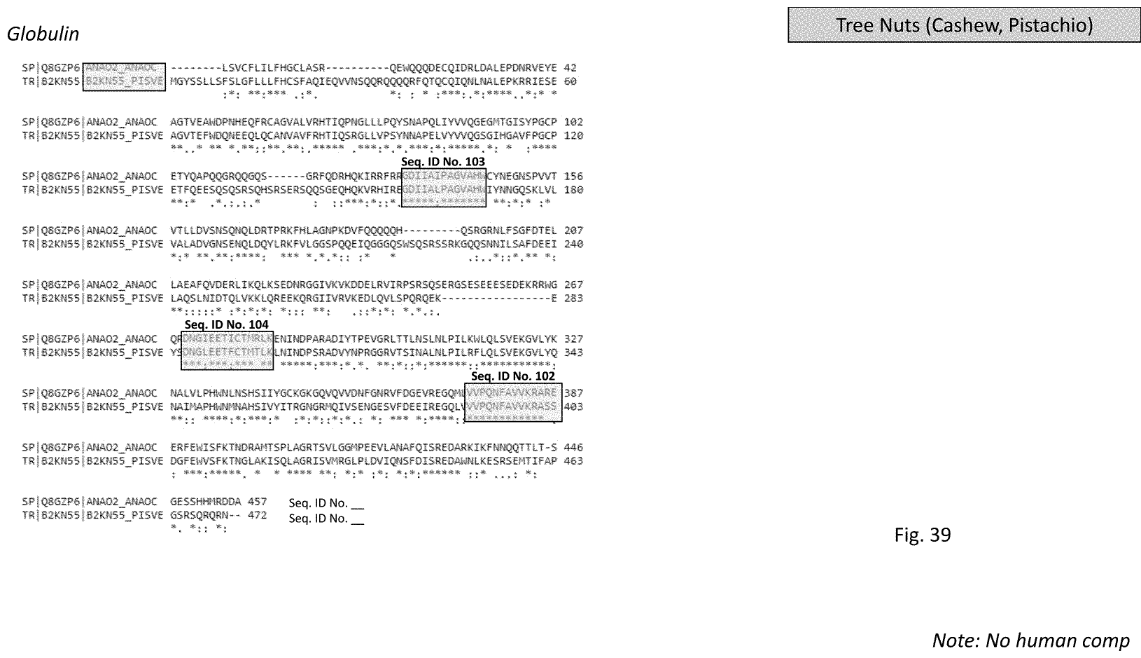

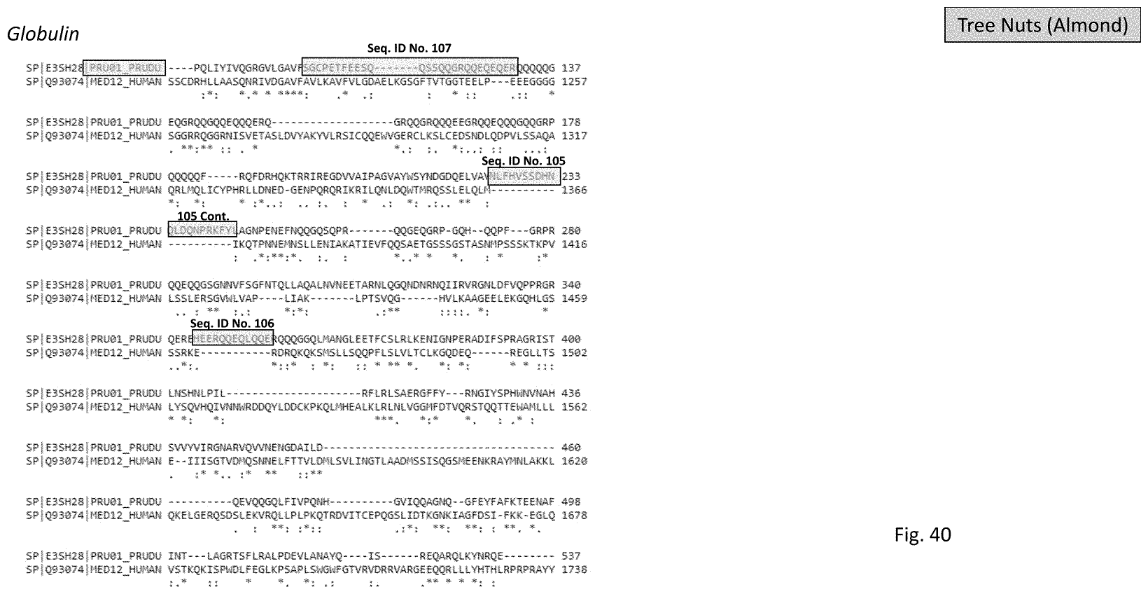

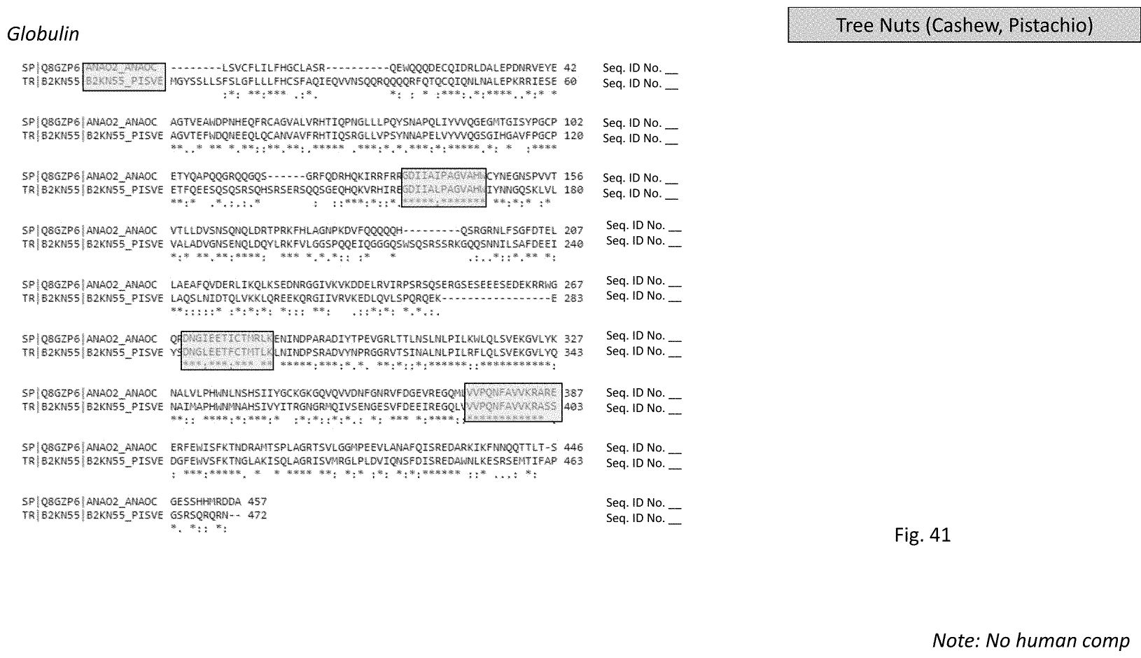

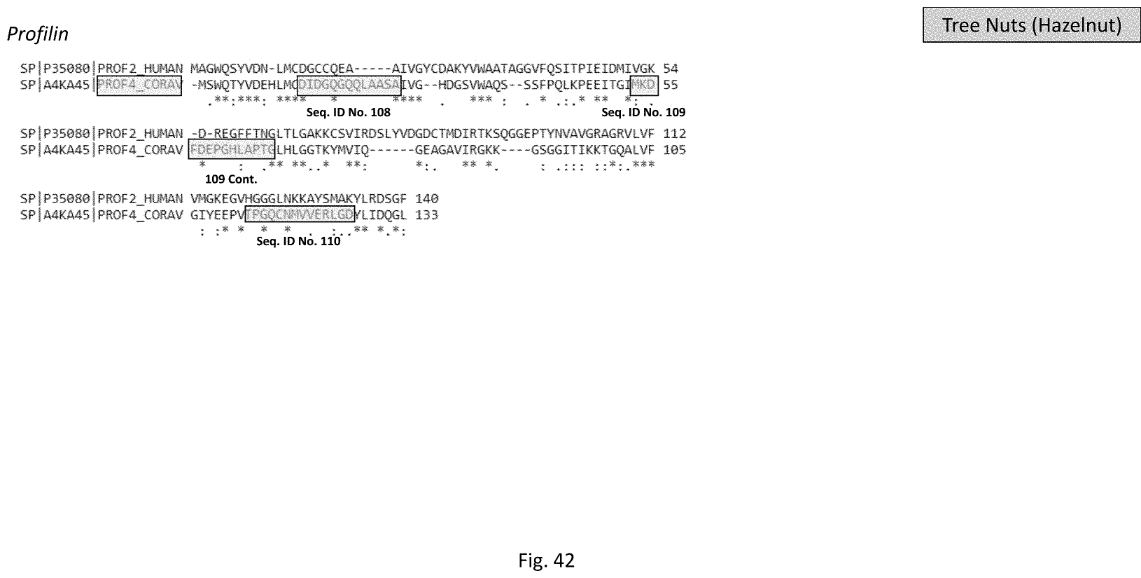

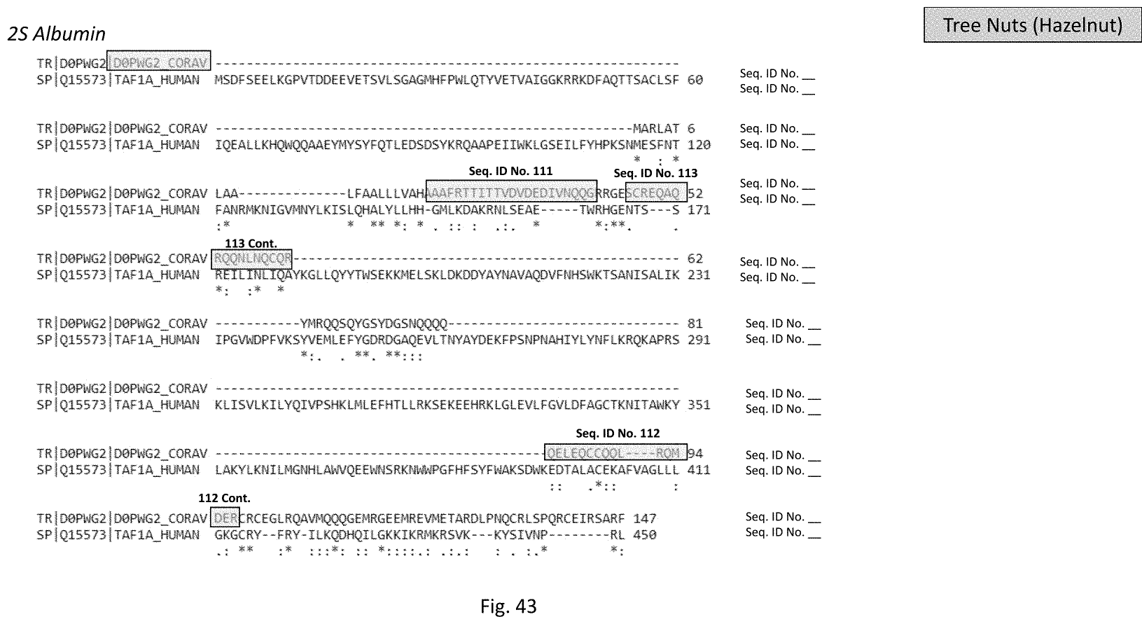

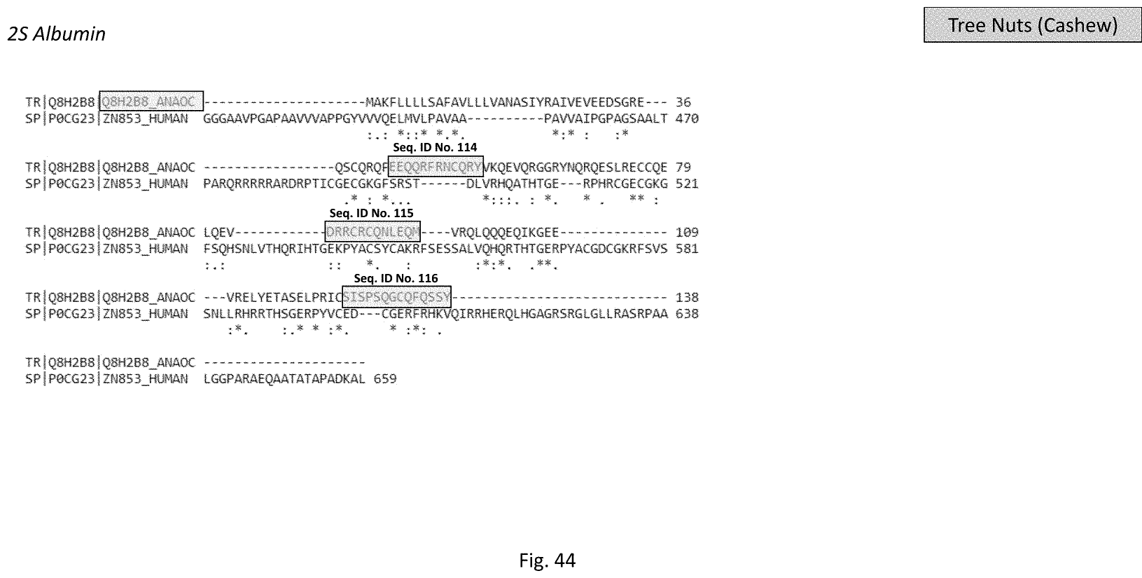

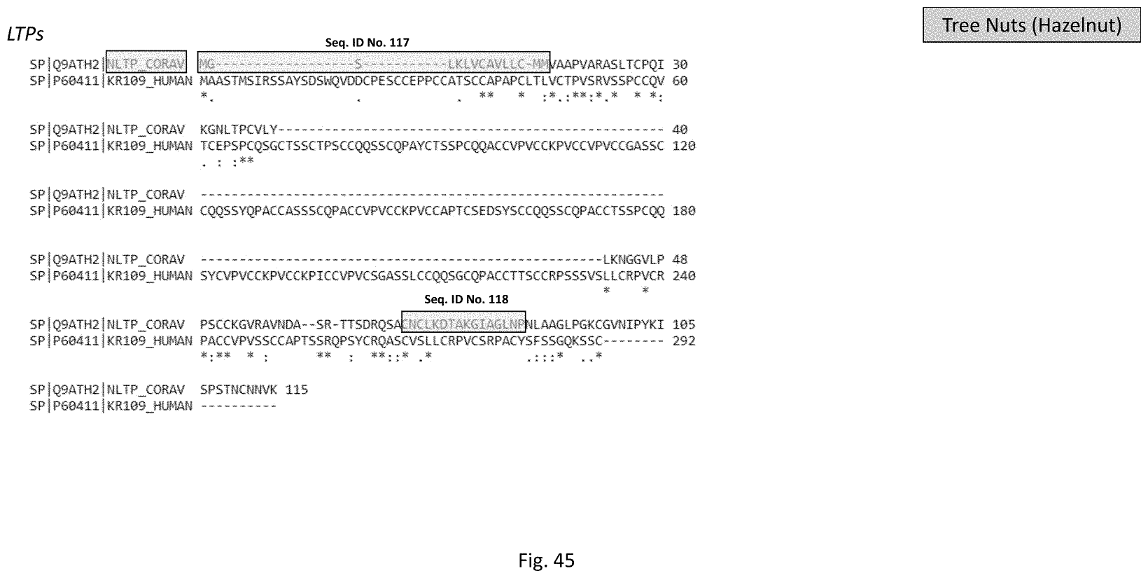

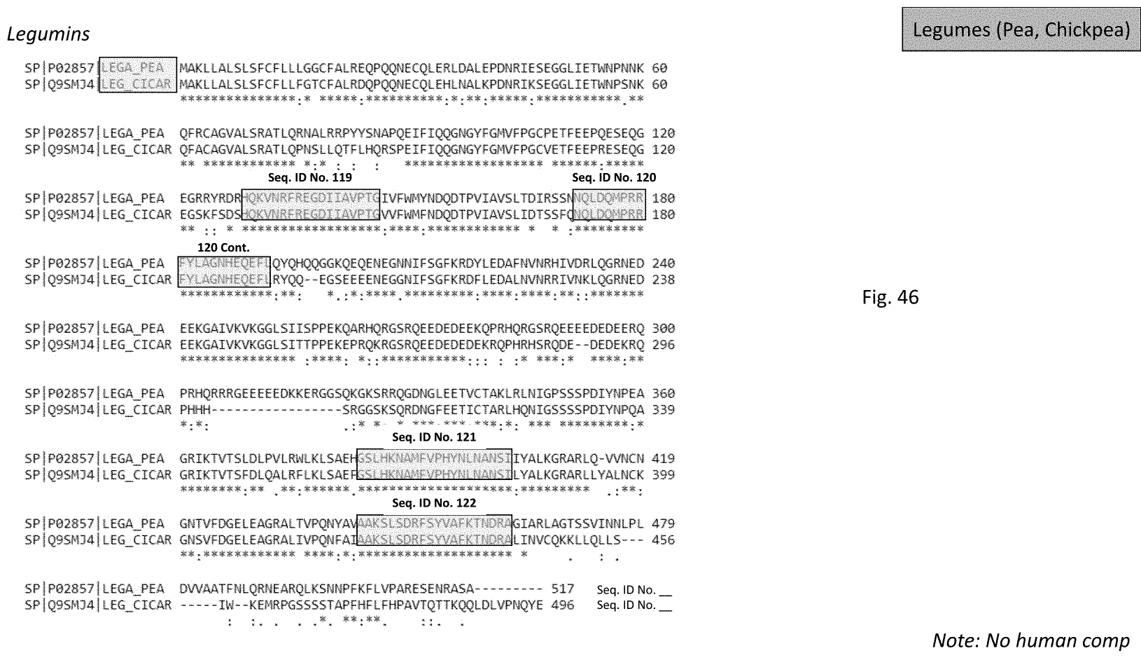

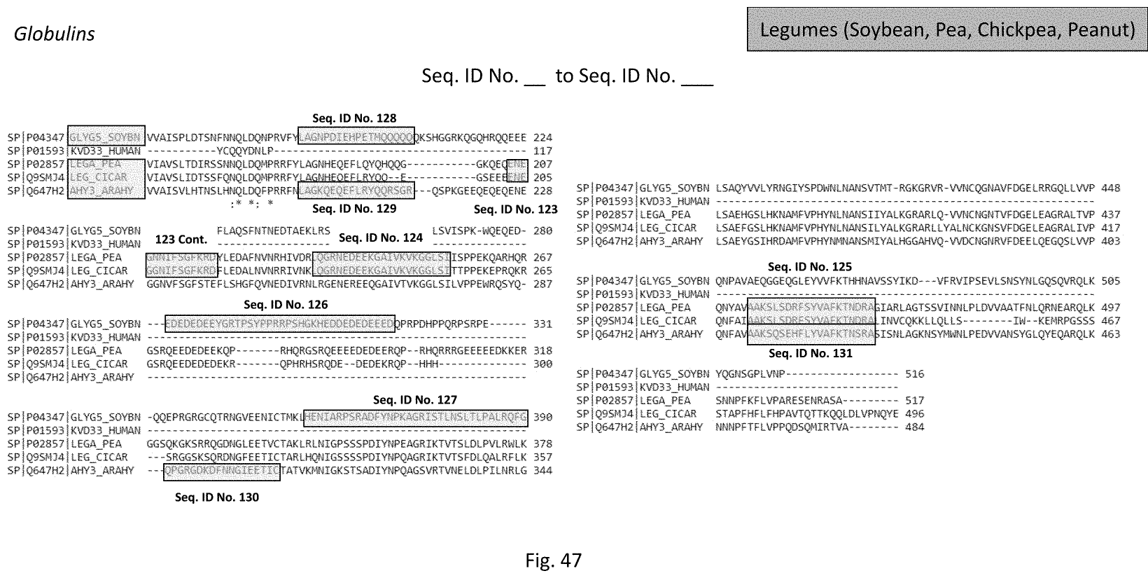

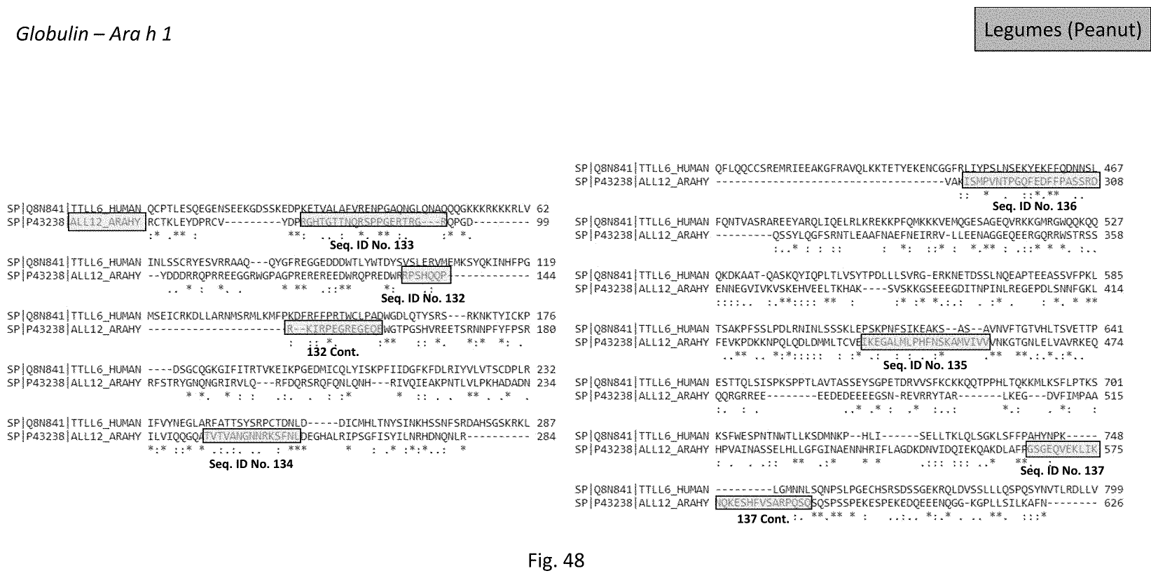

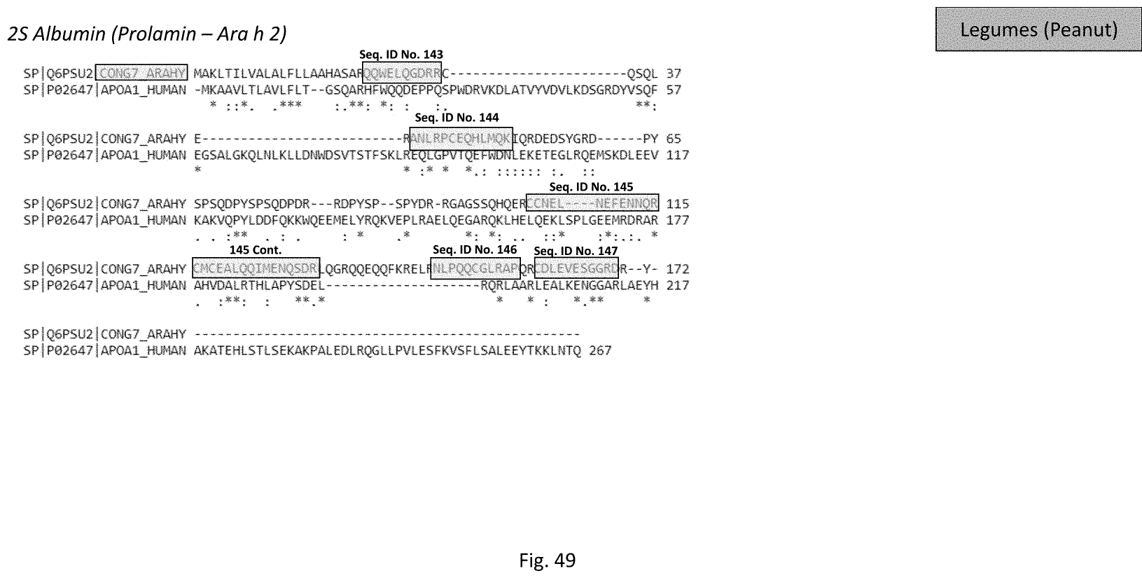

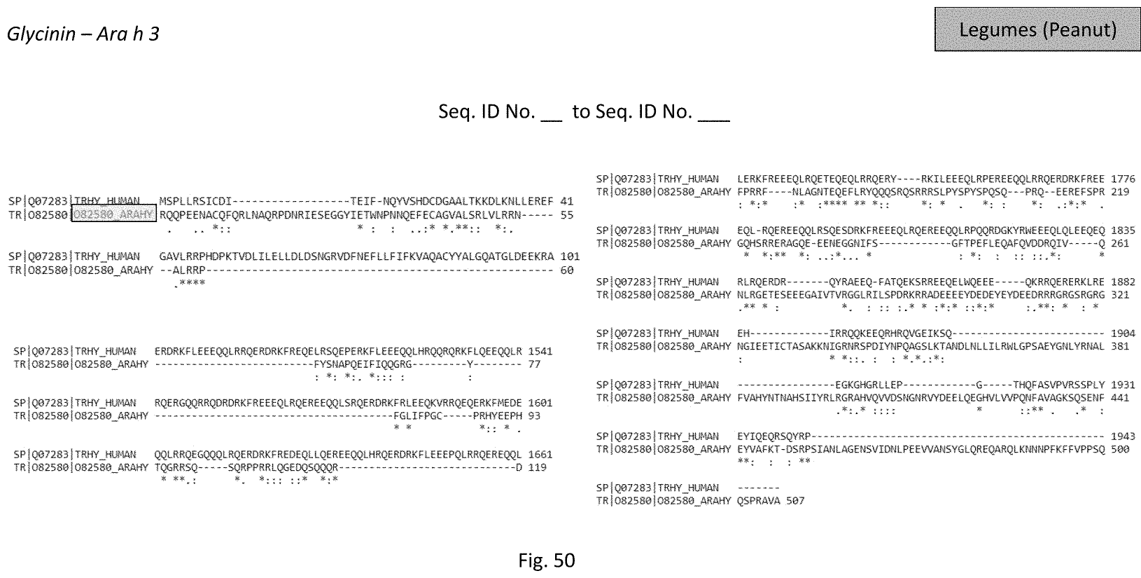

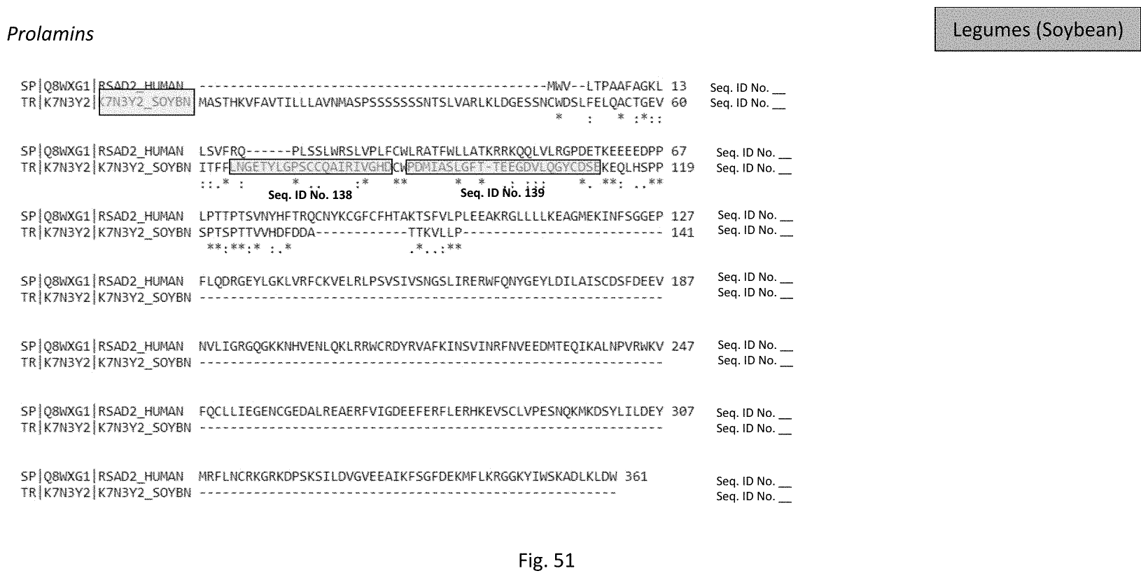

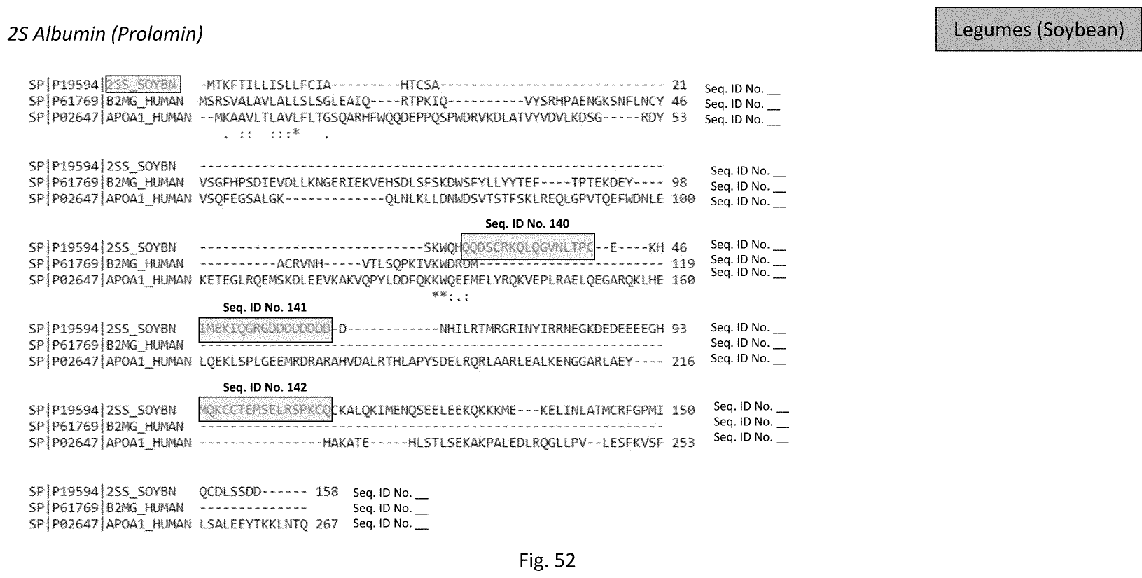

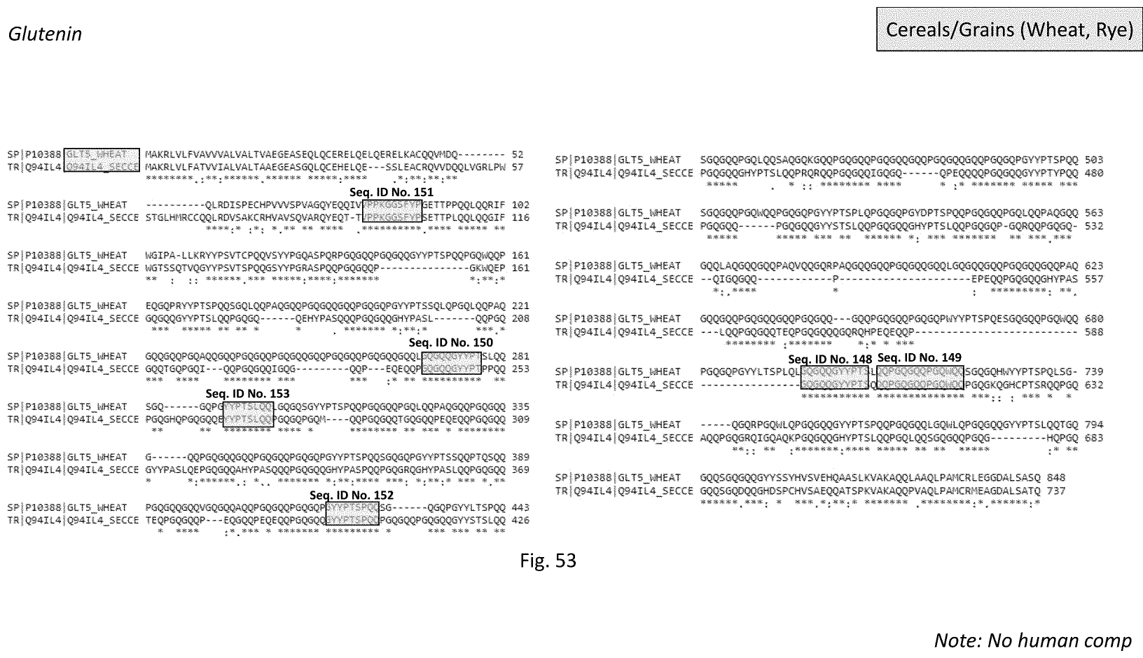

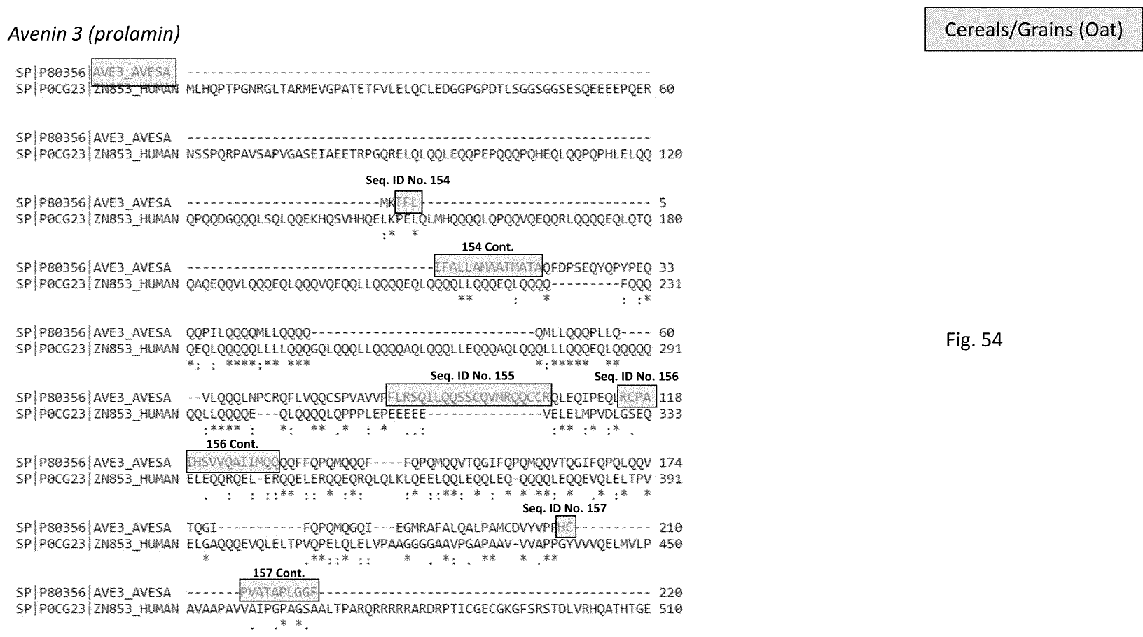

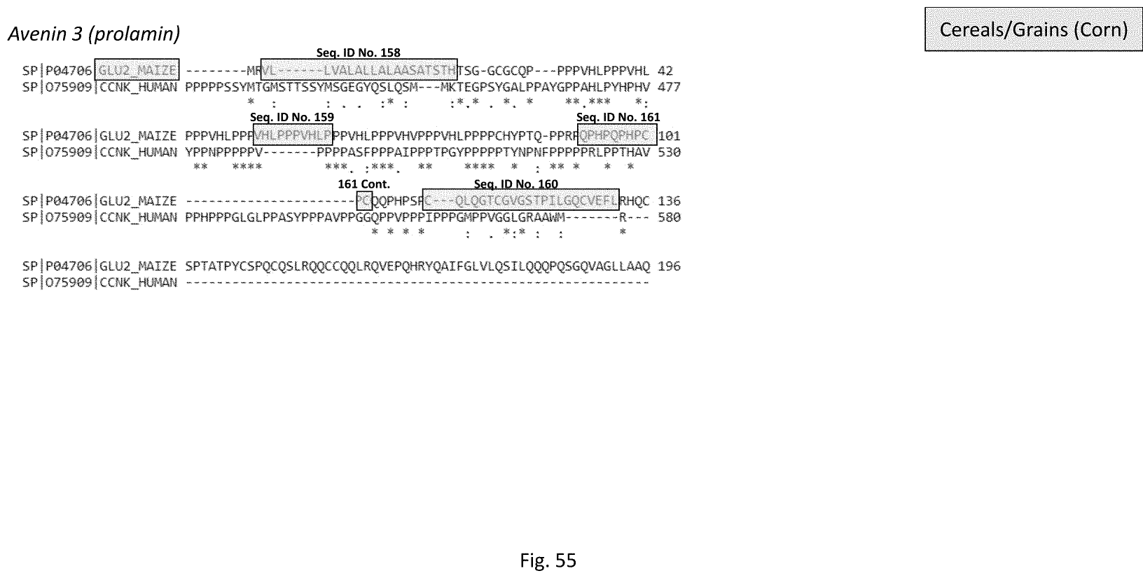

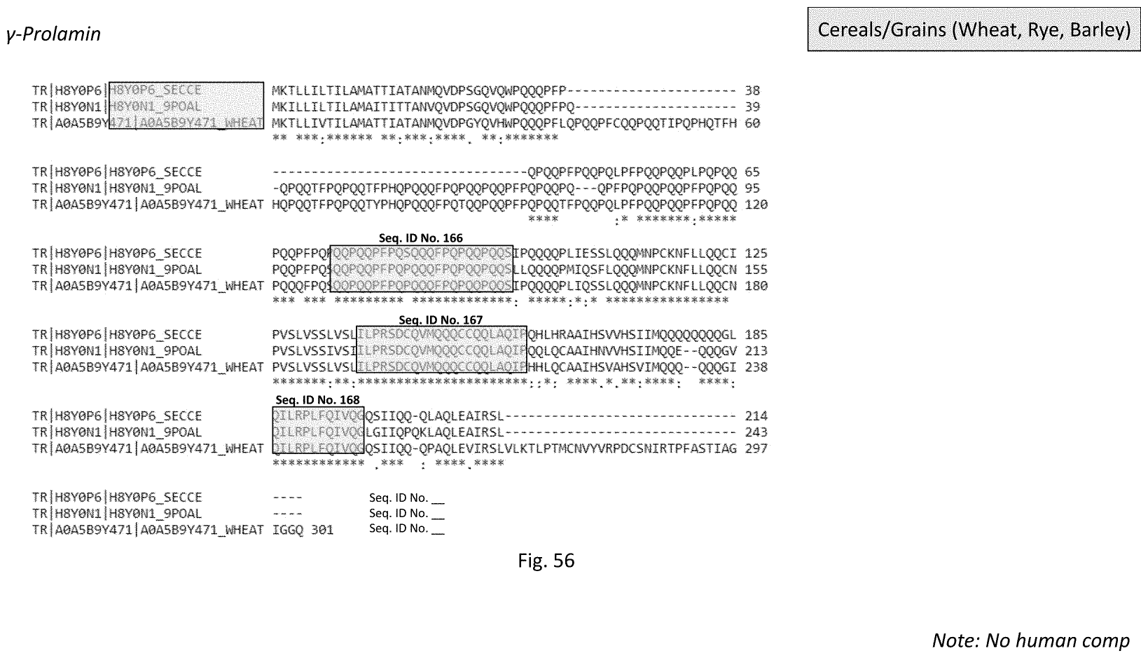

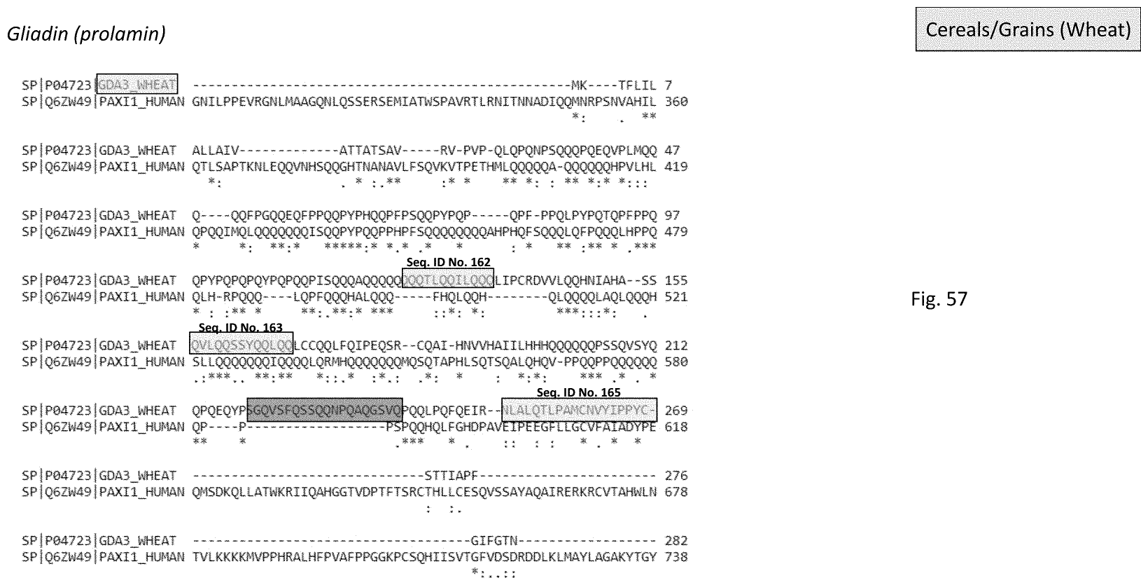

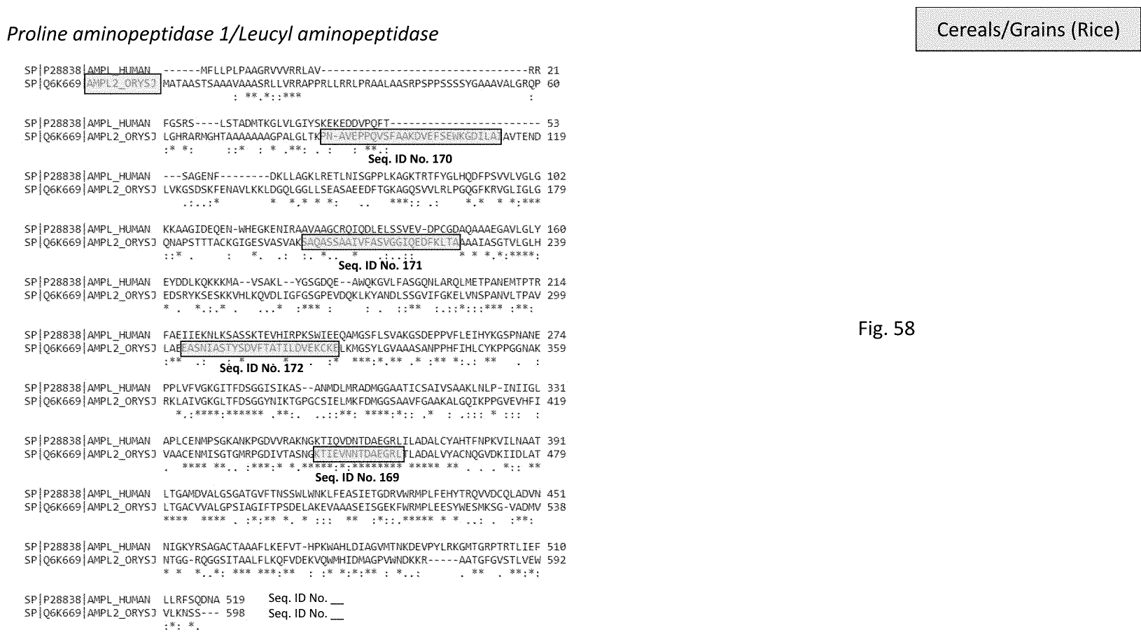

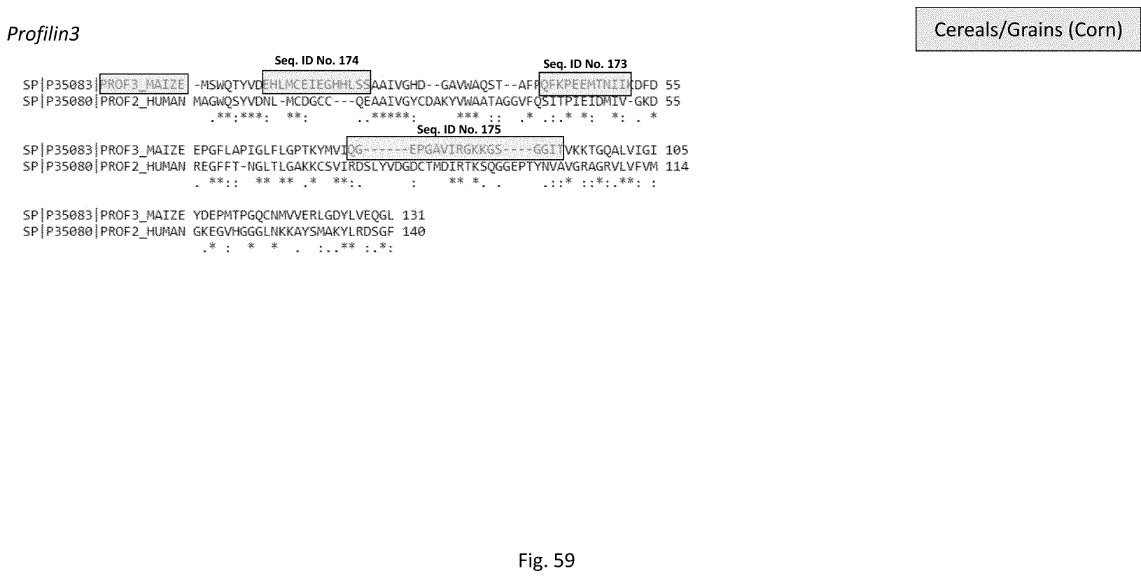

Each respective sequence fragment shown in FIGS. 8-59 includes a sequencing identification no. (Seq. Id No.) for ease of reference. The above figures are exemplary, illustrative and non-limiting of the disclosed principles.

### DESCRIPTION

In the following detailed description, numerous specific details are set forth by way of examples in order to provide a thorough understanding of the relevant teachings. It should be noted, however, that the present teachings may be practiced without such details. In other instances, known methods, procedures, or components have been described at a relatively high level, without detail, in order to avoid unnecessarily obscuring aspects of the present teachings.

An embodiment of the disclosure relates to substrates that identify, test, or perform assays on substances when the substrates are exposed to the substances. For example, a substrate of the present disclosure may be formed into a strip, which, when submersed in a liquid, may identify the presence of one or more substances or contaminants in the liquid. For example, the present disclosure includes identification of allergens, which are substances that trigger the onset of allergic reactions. Identification of allergens, especially identifying more than one known allergen at a time, is important because knowledge of the presence of allergens affects prevention, care, treatment, and decision making in the relevant fields.

An example from one subset of the relevant fields includes detection of allergens with respect to human milk fed to children. This knowledge has significant importance in that field. Approximately 80% of food allergies occur before a child's first birthday, and the chance of resolving the allergy in future years may depend on strict avoidance of the offending food. Even a small exposure to the allergen may delay the development of tolerance in a child exhibiting the allergy. Consequently, elimination of the offending food from the child's diet has a significant impact on the child. Moreover, for breast-feeding mothers, elimination of the food from the mother's diet is also often prescribed. This elimination diet can be difficult for the mother for a number of reasons, including the time delay at which point the allergen manifests in the human milk after having been consumed by the mother. More significantly, the inclusion or elimination of a particular food in the mother's diet may not always result in the presence or absence of the corresponding allergen in the human milk. Accordingly, the most accurate means in determining whether an allergen is present in the human milk, and thus the child's diet, is to test the milk itself for the allergen.

Additionally, even when the allergen associated with a food in a mother's diet does express itself in the human milk, the expression may be delayed for two weeks or more. This may be due to digestive delay in the mother's body. For this reason, if a mother is being put on an elimination diet, the child is placed on infant formula for about two weeks before the mother's human milk can be used again. This interim two-week period negatively impacts both mother and child: the child may experience infant food sensitivity and have other health issues; the mother may lose her milk supply as a result of drying of the human milk. As an additional detriment, extra financial costs are born because infant formula is expensive.

In the case of adopted children, adoptive parents who adopt a food-sensitive child and wish to receive donated human milk to feed the child experience additional challenges. The parents have no way of determining what substances were consumed by the milk-donating mother prior to pumping human milk and thus have no knowledge of what allergens may exist in the milk. Currently, there are no systems in place for such parents to analyze the milk for allergen or contaminants Therefore, if the child presents with an allergy, for example a dairy allergy, then receiving donated human milk is no longer an option at all. This is true even though it is scientifically proven that human milk is a higher caliber nutrition for the infant than that of formula. This problem is also shared by mother's who are unable to produce milk and want to receive or purchase human milk from others. The practice of donating and receiving human milk is a large and expanding industry that will continue to face such challenges.

These are but a few examples of many which illustrates the acute need for a substrate that identifies substances, including allergens, by exposure to them. While the above examples are based on the needs associated with detection of allergens in human milk, the disclosed principles are not limited to this field and have much more expansive breadth.

For example, the present disclosure can be used to detect allergens in cow milk, sheep milk, or milk from other mammals, both for use by humans and for use by their own offspring. Additionally, the present disclosure can be used to detect substances other than allergens. For example, persistent organic pollutants (POPs), heavy metals, pesticides, and other contaminants. Moreover, the substrate of the present disclosure may be used to detect allergens in other substances other than human milk. For example, drinking water, infant formula, and alternative milks which claim to be free of specific allergens. The substrate of the present disclosure may be used with other liquids as well, including bodily fluids or consumable fluids. Additionally, the substrate of the present disclosure may be used in conjunction with gases, instead of liquids, and be used to determine the presence of one or more substances or contaminants in a gas when the substrate is exposed to the gas.

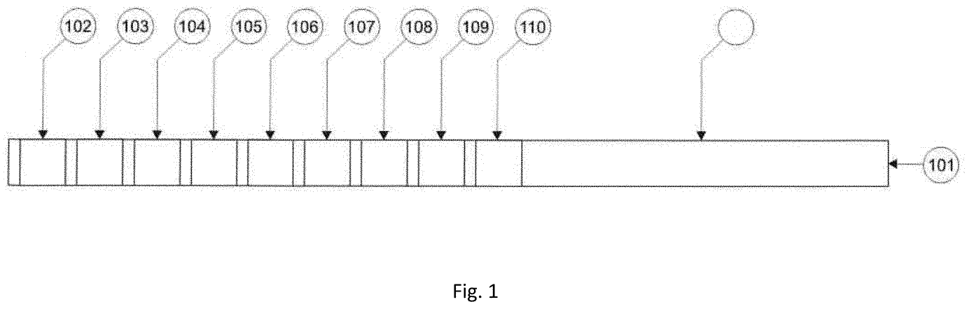

With reference to FIG. 1, a substrate 100 is shown. Substrate 100 includes a base 101 and detection sites 102 through 110. Functionally similar parts are depicted with the same reference numbers in all of the figures.

The base 101 is shown to be a relatively flat rectangular strip in FIG. 1. Nevertheless, it is not limited as such. Base 101 and substrate 100 may be formed in other shapes and geometries. FIG. 4 provides an alternative example of substrate 100 where bases 101 which is in the form of a cylinder. For example, substrate 100 may be round or of any polygonal shape. It may also be configured as a ring, sphere, cube, or with any desired geometry. The geometry of the substance or the base may be chosen arbitrarily, based on manufacturing considerations, based on availability of stock material for use as the substrate, or based on considerations relating to use, including ease of identification, efficacy of packaging and handling, etc. In summary, the substrate, and correspondingly the base, may be created in any size, shape, and geometry that is suitable for the particular application.

Base 101 may be made from any suitable material including paper or wood byproducts, plastic or polymers, organic matter such as a fabric, woven material, cellulose, cutin, tannin-based material, metal, ceramic, or vinyl. One available and inexpensive option may include paper, such as to allow for maximum liquid absorption with clear result readings. Other examples, in addition to the material named above, may include a matrix of paper or plastic, for example, or any combination of the above-mentioned materials.

Detection sites 102 through 110 are detection sites on a surface of the substrate 100 that will change, in physical attribute, when brought into contact with a specific substrate to which they are reactive. That is, the presence of a particular offending substance will result in the physical change in portion 102; thereby enabling detection of the offending substance by observing the change in portion 102.

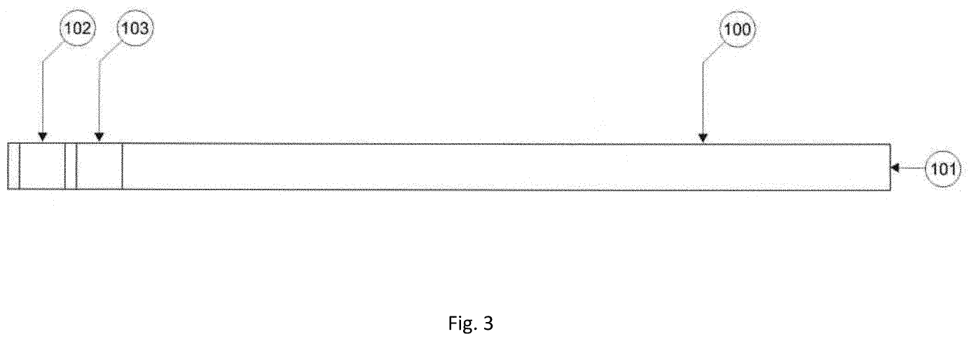

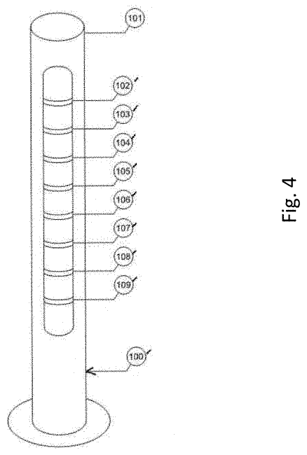

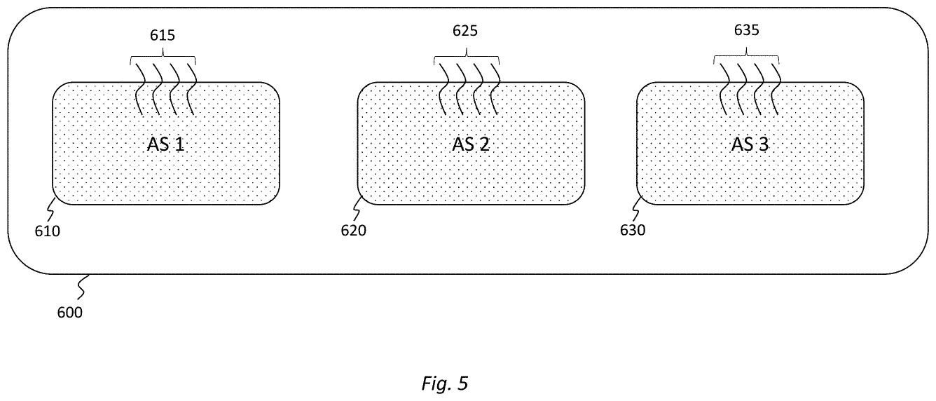

By way of example, portion 102 may be constructed to react to certain substances or contaminants. One class of an allergens or contaminants is allergen proteins or their derivatives and their corresponding detection antibodies. By way of example, when portion 102 is brought into contact with, or otherwise exposed to, the liquid or gas containing the allergen proteins or their antibodies, it's color may change. One example of a color change may be from white to pink. Naturally, the disclosure is not limited to this example. Rather portion 102, as well as any of the other detection sites, may be constructed to be reactive to a desired offending substance. Additionally, the physical manifestation of the reaction can be in any selected form such as a change in color, odor, texture, or fluorescence. Further, the number of detection sites on substrate 100 need not be 9, as depicted in FIG. 1 with respect to detection sites 102-110. Therefore, the substrate 100 may have one or two or many detection sites such as portion 102. FIG. 3 shows an example of substrate 100 with only two detection sites.

The number of detection sites depends on the usage and can be customized for various considerations including the number of substances desired to be identified simultaneously, the potential number of substances present, the cost of the strip, the durability and longevity of one portion (reactive to one substance) as compared with another portion (reactive to another substance).

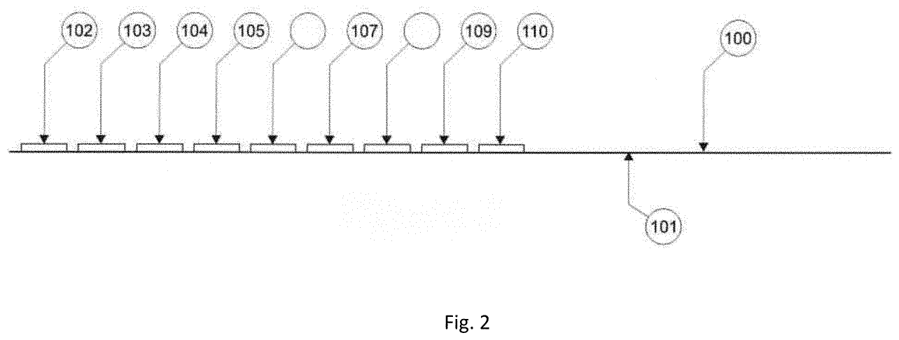

In the embodiment shown in FIG. 1, detection sites 102-110 are contemplated to be of a different compound each of which is reactive to a different substance. Specifically, detection sites 102-110 may define active sites or detection sites. That is, in the embodiment of FIG. 1, substrate 100 can detect simultaneously 9 different substances in a particular material. For example, if the material was human milk and substrate 100 was immersed in the human milk, it could detect up to 9 different allergens in the human milk simultaneously. That is, substrate 100 would test for 9 different allergens simultaneously. The embodiment of FIG. 2 is a side view of the substrate 100 as shown in FIG. 1.

The number of detection sites is arbitrary and may be subject to the intended use. For example, substrate 100 may include 2, 4 or 20 different active sites. In one embodiment, each active site may be configured to detect a different target molecule. In another embodiment, multiple active sites may be used to detect presence of a single target molecule. In still another embodiment, multiple active sites may be used to detect presence the degree of presence of a single target molecule.

The dimensions (width, length and thickness) of the active sites 102-110 may be substantially identical or may vary depending on the desired application. For example, at least one of the active sites may be configured to provide a larger surface area so as to increase the exposure area for the desired target.

FIG. 3 depicts a flat substrate with only two detection sites 102-103. Detection sites 102 and 103 are substantially the same to provide redundancy in the event that one portion does not function properly. Accordingly, in the embodiment of FIG. 3, the substrate 100 tests for a single substance with both or either detection sites 102 and 103. As stated earlier, the number of detection sites, the degree of redundancy, and whether or not a control portion is provided depend on the particular application and may be selected to customize the substrate of the present disclosure for the particular use.

In the embodiment of FIG. 4, the base 101′ is formed in the shape of a cylinder and substrate 100′ is capable of standing upright. Detection sites 102′-109′ (however many are chosen for inclusion) may be placed on the inner surface of base 101′, that is, inside the cylinder. The medium to be tested is placed inside the cylinder so as to come into contact with detection sites 102′-109′, thereby allowing detection of allergens or contaminants. The cylinder may be opaque so that inspection of detection sites 102′-109′ may be performed by looking into the cylinder. It may alternatively be transparent so that inspection can be performed visually from the exterior of the cylinder. In yet another alternative configuration, the cylinder may have a clear window portion on the cylinder formed in front of detection sites 102′-109′ so that detection sites 102′-109′ may be readily viewable from the exterior of the cylinder. Additionally, the cylinder may be, but need not be, formed from a material that is readily tearable, such as paper, so that when a test is complete, the cylinder may be ripped open and laid flat to inspect detection sites 102′-109′. In this latter embodiment, a rip cord or seam for easy separation may be included in the base 101′.

In yet another embodiment with a similar shape as shown in FIG. 4, the base 101′ may be constructed of several single strips (such as the base depicted in FIG. 3) which are connected together along their edges. Each single strip may include a single (or redundant) detection sites for identifying a single allergen. In this latter configuration, the strips are joined to form the cylinder-like body and the detection sites 102′-109′ will be positioned in a circle on the inside surface of the cylinder. Alternatively, the detection sites may be offset so that each is at a different height with respect to the bottom of the cylinder and the active areas 102′-109′ form a spiral on the surface of the cylinder.

As stated, the disclosure is not limited for detection of allergens or contaminants in human milk, or even milk, and can be configured to detect any desired number of substances in any desired material. Additionally, even if multiple detection sites were included on one substrate, they need not each detect a different substance—they can all detect the same substance (for example be redundant similar to the embodiment of FIG. 3). Moreover, any combination of number of detection sites and detection capability is contemplated by this disclosure. For example, two detection sites can be included for detecting substance ‘A’ and three detection sites can be included for detection of substance ‘B’ and so on. Detection sites may also be distributed on the surface according to any geometry. In one embodiment, they can be equally spaced on one end of a strip as shown in FIG. 1. They may also be spread across the entire substrate, may be equidistance or not, may be redundantly formed at each edge of the substrate, may be redundantly formed on one edge of the substrate, etc. If the substrate has an alternative geometry, detection sites can be formed on an inside surface, outside surface, on a single surface, on multiple surfaces, redundantly or not, and in any configuration desired. In another example, where the substrate may be in the shape of a cube, multiple detection sites, each reactive to a particular substrate, can be formed on a top surface of the cube and the same configuration of multiple detection sites can be redundantly formed on the remaining three sides, leaving two sides free from the multiple detection sites.

Detection sites 102-110 (including of course detection sites 102′-109′) may be constructed of any compound that detects a particular substance or group of substances as desired. One class of compounds that detect substances include imprinted polymers which may detect specific proteins. Therefore, if a portion is created from a specific imprint polymer, it is capable of detecting the respective protein. Other examples for detection of allergens in human milk include antibodies and magnetic particles.

In an example using the configuration as shown in the embodiment of FIG. 1, the detection sites 102-109 can be constructed to detect the presence of the top eight allergens in human milk which may include: egg, dairy, soy, peanut, tree nut, fish, shellfish, and wheat. Portion 110 can be left inert as a blank or a control. Additionally, if the substrate is used in communities or countries that identify a different number of relevant allergens, the number of detection sites can be adjusted to test for all of those—for example, the substrate can test for 12 allergens. Moreover, it is understood that the identification of allergens may change over time and thus detection sites would obviously be configured to detect for the relevant allergens. These variations are equally applicable to the other embodiments disclosed herein, including the embodiment of FIG. 4.

Naturally, the disclosure is not limited to any particular type or number of allergens. For example, this disclosure could detect as few as a single allergen or any combination of multiple allergens. And as explained above and further expounded below, use of the substrate with human milk for detection of allergens is but one example of its configuration and use to which it is not limited. Other consumable liquids which may benefit from testing using the present disclosure include, but are not limited juice, water, broth, reconstituted powders into liquid (such as protein powder), concentrates, alcoholic beverages, and carbonated drinks Where various liquids may have higher or lower pH, to the extent necessary to adjust the pH to a more neutral level before testing, a test kit which permits adjustment of the pH in preparation for testing may be provided with the substrate.

The ability to test multiple liquids may be made possible through the particular choice of substrate and the composition of portion. It may alternatively be accomplished by use of an extraction buffer system. An extraction buffer may allow the user to more effectively detect the presence of substances or contaminants within the medium being tested by increasing detection levels to lower amounts (for example a smaller parts per million amount) than practical without use of the extraction buffer.

In operation, substrate 100 (or 100′) is exposed to the medium to be analyzed (for example, human milk) for the presence of a substance (for example, an allergen). The substrate is brought into contact with the material. If the material is a liquid, substrate 100 may be immersed or submersed in the liquid. If the material is a gas or a solid, the substrate may be brought into contact with the material such detection sites, e.g., 102-110, are directly in contact with the medium. In the case of substrate 100′ the substance is placed inside the cylinder such that detection sites 102′-109′ are directly in contact with the medium. Once detection sites are brought into contact with the medium, they will identify the presence of the substances to which they react if the substances are present. Accordingly, the substrate may be used to detect various substances.

The present disclosure has been described using some examples as illustrated above. In practice, the disclosure may be formed as one or more substrates which may be provided or sold individually to consumers. Alternatively, one or more of the aspects of the disclosure can be packaged together and sold collectively. Moreover, the present disclosure may be formed into kits which include one or more of the elements of the disclosure included in a kit and provided to consumers. Such kits may be formed with various alternative embodiments for aspects of the disclosure so that the consumer can select and customize their detection kits. The present disclosure may be scaled as noted above and any corresponding kit may include different sizes of components and potentially multiple buffering agents. In summary, the aspects of the disclosure described herein can be adjusted, scaled, and configured as described herein to provide a maximally customizable system or kit.

### Substrate

In one embodiment may comprise a rigid or flexible substrate configured to receive one or more detection areas. In certain embodiments substrate 100 (FIG. 1) may comprise fibrous material such as paper, cardboard and the like. In other embodiments, substrate 100 (FIG. 1) may comprise polymeric material. In still another embodiment, substrate 100 (FIG. 1) may comprise silicate material such as glass. The substrate may be selected based on its ability to receive one or more probes at least one detections site.

The substrate may be prepared prior to coupling probes thereon, for example, by washing with purified water, solvent, crosslinking agent, functional monomer, polymerization initiator or a surface activating agent.

### Probes

In certain applications, one or more molecular probes may be coupled to the active site (e.g., 102, FIG. 1) of substrate 100 (FIG. 1) to attract and bind a target molecule. In an exemplary embodiment the probe may comprise a binding site. In another embodiment, a portion or all of the probe may be used to bind to the target molecule. The binding site may be selected to at least one of attract and/or bind the probe to the target molecule. Thus, the binding site can be functionally coupled to the probe at one end and to the target molecule at the other end.

In one embodiment, the probe may comprise a molecule having an active binding site configured to attract and bind to a target molecule. An exemplary probe may comprise a molecular chain, a polymer, a protein epitope or an amino acid chain. In further example, a 14-protein epitope for cow's milk protein P-Lactoglobulin found in human milk is sequence TPEVDDEALEKFDK. An exemplary and non-exhaustive, protein epitope list in TABLE 1 below.

TABLE 1
Protein epitope list

| | Annotated Sequence in | # | | | Found in |
|---|---|---|---|---|---|
| Source | Human Milk (Zhu, 2019) | PSMs | Protein Names | Organisms | 6 Samples |
| Cow’s | [R].FFVAPFPEVFGKEK.[V] | 110 | Alpha-S1-casein | Bos taurus | 6 |
| Milk | | | | | |
| Protein | | | | | |
| Cow’s | [K].HIQKEDVPSER.[Y] | 69 | Alpha-S1-casein | Bos taurus | 6 |
| Milk | | | | | |
| Protein | | | | | |
| Cow’s | [K].HPIKHQGLPQEVLNENLLR[F] | 41 | Alpha-S1-casein | Bos taurus | 6 |
| Milk | | | | | |
| Protein | | | | | |
| Cow’s | [K].HQGLPQEVLNENLLR.[F] | 28 | Alpha-S1-casein | Bos taurus | 6 |
| Milk | | | | | |
| Protein | | | | | |
| Cow’s | [K].EGIHAQQKEPMIGVNQELAY | 26 | Alpha-S1-casein | Bos taurus | 6 |
| Milk | FYPELFR.[Q] | | | | |
| Protein | | | | | |
| Cow’s | [K].EKVNELSK.[D] | 19 | Alpha-S1-casein | Bos taurus | 6 |
| Milk | | | | | |
| Protein | | | | | |
| Cow’s | [R].FFVAPFPEVFGK.[E] | 12 | Alpha-S1-casein | Bos taurus | 6 |
| Milk | | | | | |
| Protein | | | | | |
| Cow’s | [R].LHSMKEGIHAQQK.[E] | 8 | Alpha-S1-casein | Bos taurus | 6 |
| Milk | | | | | |
| Protein | | | | | |
| Cow’s | [K].EGIHAQQKEPMIGVNQELAY | 6 | Alpha-S 1-casein | Bos taurus | 6 |
| Milk | FYPELFR.[Q] | | | | |
| Protein | | | | | |
| CMP or | [R].YLGYLEQLLR.[L] | 104 | Alpha-S1-casein | Bos taurus | 6 |
| Egg | | | | and Ovis aries | |
| CMP or | [K].EDVPSER.[Y] | 19 | Alpha-S 1-casein | Bos taurus | 6 |
| Egg | | | | and Ovis aries | |
| CMP or | [K].TTMPLW.[-] | 19 | Alpha-S1-casein | Bos taurus | 6 |
| Egg | | | | and Ovis aries | |
| CMP or | [K].TTMPLW.[-] | 8 | Alpha-S1-casein | Bos taurus | 6 |
| Egg | | | | and Ovis aries | |
| Cow’s | [K].FALPQYLK.[T] | 57 | Alpha-S2-casein | Bos taurus | 6 |
| Milk | | | | | |
| Protein | | | | | |
| Cow’s | [R].NAVPITPTLNR.[E] | 29 | Alpha-S2-casein | Bos taurus | 6 |
| Milk | | | | | |
| Protein | | | | | |
| Cow’s | [K].AMKPWIQPK.[T] | 20 | Alpha-S2-casein | Bos taurus | 6 |
| Milk | | | | | |
| Protein | | | | | |
| Cow’s | [K].TKVIPYVR.[Y] | 16 | Alpha-S2-casein | Bos taurus | 6 |
| Milk | | | | | |
| Protein | | | | | |
| Cow’s | [K].AMKPWIQPK.[T] | 2 | Alpha-S2-casein | Bos taurus | 6 |
| Milk | | | | | |
| Protein | | | | | |
| Cow’s | [K].TKVIPYVRYL[-] | 1 | Alpha-S2-casein | Bos taurus | 5 |
| Milk | | | | | |
| Protein | | | | | |
| CMP | [K].ITVDDKHYQK.[A] | 18 | Alpha-S2-casein | Bos taurus | 6 |
| or Egg | | | | and Ovis aries | |
| CMP | [K].ALNEINQFYQK.[F] | 16 | Alpha-S2-casein | Bos taurus | 6 |
| or Egg | | | | and Ovis aries | |
| CMP | [K].LTEEEKNRLNFLK.[K] | 1 | Alpha-S2-casein | Bos taurus | 6 |
| or Egg | | | | and Ovis aries | |
| | | | | and Sus scrota | |
| Cow’s | [K].AVPYPQR.[D] | 24 | Beta-casein | Bos taurus | 6 |
| Milk | | | | | |
| Protein | | | | | |
| Cow’s | [K].VKEAMAPK.[H] | 22 | Beta-casein | Bos taurus | 6 |
| Milk | | | | | |
| Protein | | | | | |
| Cow’s | [R].GPFPIIV.[-] | 12 | Beta-casein | Bos taurus | 6 |
| Milk | | | | | |
| Protein | | | | | |
| Cow’s | [K].VLPVPQKAVPYPQR.[D] | 4 | Beta-casein | Bos taurus | 6 |
| Milk | | | | | |
| Protein | | | | | |
| CMP or | [K].VLPVPQK.[A] | 164 | Beta-casein | Bos taurus | 6 |
| Egg | | | | and Ovis aries | |
| CMP or | [K].HKEMPFPK.[Y] | 9 | Beta-casein | Bos taurus | 6 |
| Egg | | | | and Ovis aries | |
| CMP or | [K].HKEMPFPK.[Y] | 3 | Beta-casein | Bos taurus | 6 |
| Egg | | | | and Ovis aries | |
| Cow’s | [R].SPAQILQWQVLSNTVPAK.[S] | 64 | Kappa-casein | Bos taurus | 6 |
| Milk | | | | | |
| Protein | | | | | |
| Cow’s | [R].FFSDKIAK.[Y] | 21 | Kappa-casein | Bos taurus | 6 |
| Milk | | | | | |
| Protein | | | | | |
| Cow’s | [R].YPSYGLNYYQQKPVALINNQ | 1 | Kappa-casein | Bos taurus | 5 |
| Milk | FLPYPYYAKPAAVR.[S] | | | | |
| Protein | | | | | |
| CMP or | [K].YIPIQYVLSR.[Y] | 54 | Kappa-casein | Bos taurus | 6 |
| Egg | | | | and Ovis arias | |
| Cow’s | [K].AGLCQTFVYGGCR.[A] | 44 | Pancreatic | Bos taurus | 6 |
| Milk | | | trypsin inhibitor | | |
| Protein | | | | | |
| Cow’s | [R].NNFKSAEDCMR.[T] | 8 | Pancreatic | Bos taurus | 6 |
| Milk | | | trypsin inhibitor | | |
| Protein | | | | | |
| Cow’s | [R].IIRYFYNAK.[A] | 1 | Pancreatic | Bos taurus | 6 |
| Milk | | | trypsin inhibitor | | |
| Protein | | | | | |
| Cow’s | [R].TPEVDDEALEKFDK.[A] | 19 | Beta-lactoglobulin | Bos taurus | 6 |
| Milk | | | | | |
| Protein | | | | | |
| Cow’s | [R].VYVEELKPTPEGDLEILLQK. | 4 | Beta-lactoglobulin | Bos taurus | 6 |
| Milk | [W] | | | | |
| Protein | | | | | |
| Cow’s | [R].TPEVDDEALEK.[F] | 1 | Beta-lactoglobulin | Bos taurus | 5 |
| Milk | | | | | |
| Protein | | | | | |
| CMP or | [K].ALPMHIR.[L] | 5 | Beta-lactoglobulin | Bos taurus | 6 |
| Egg | | | | and Ovis aries | |
| CMP or | [K].VLVLDTDYKK.[Y] | 3 | Beta-lactoglobulin | Bos taurus | 6 |
| Egg | | | | and Ovis aries | |
| CMP or | [K].TKIPAVFK.[I] | 2 | Beta-lactoglobulin | Bos taurus | 6 |
| Egg | | | | and Ovis aries | |
| CMP or | [K].ALPMHIR.[L] | 1 | Beta-lactoglobulin | Bos taurus | 6 |
| Egg | | | | and Ovis aries | |
| Cow’s | [K].LGSVYTEGGFVEGVNKK.[L] | 14 | Bile salt- | Bos taurus | 6 |
| Milk | | | activated lipase | | |
| Protein | | | (Fragment) | | |
| Cow’s | [K].RAISQSGVGLCPWAIQQDPL | 3 | Bile salt- | Bos taurus | 6 |
| Protein | FWAK.[R] | | activated lipase | | |
| | | | (Fragment) | | |
| Cow’s | [R].CMLDRNEDMLITGGRHPFLA | 12 | Xanthine | Bos taurus | 6 |
| Milk | R[Y] | | dehydrogenase/ | | |
| Protein | | | oxidase | | |
| Cow’s | [R].NQPEPTVEEIEDAFQGNLCR | 4 | Xanthine | Bos taurus | 6 |
| Milk | [C] | | dehydrogenase/ | | |
| Protein | | | oxidase | | |
| Cow’s | [R].CMLDRNEDMLITGGRHPFLA | 3 | Xanthine | Bos taurus | 6 |
| Milk | R[Y] | | dehydrogenase/ | | |
| Protein | | | oxidase | | |
| Cow’s | [R].CMLDRNEDMLITGGRHPFLA | 3 | Xanthine | Bos taurus | 6 |
| Milk | R[Y] | | dehydrogenase/ | | |
| Protein | | | oxidase | | |
| Cow’s | [K].LGCGEGGCGACTVMLSKYD | | Xanthine | Bos taurus | 6 |
| Protein | RLQDK.[I] | | dehydrogenase/ | | |
| | | | oxidase | | |
| Cow’s | [R].VFVQKEILDQFTEEVVKQTQ | 11 | 4- | Bos taurus | 6 |
| Milk | R.[I] | | trimethyl- | | |
| Protein | | | aminobutyraldehyde | | |
| | | | dehydrogenase | | |
| Cow’s | [R].VFVQKEILDQFTEEWK.[Q] | 3 | 4- | Bos taurus | 6 |
| Milk | | | trimethyl- | | |
| Protein | | | aminobutyraldehyde | | |
| | | | dehydrogenase | | |
| Cow’s | [K].EILDQFTEEVVK.[Q] | 1 | 4- | Bos taurus | 6 |
| Milk | | | trimethyl- | | |
| Protein | | | aminobutyraldehyde | | |
| | | | dehydrogenase | | |
| Cow’s | [R].VIATFTCSGEKEVNLAVQDA | 1 | 4- | Bos taurus | 1 |
| Milk | K.[A] | | trimethyl- | | |
| Protein | | | aminobutyraldehyde | | |
| | | | dehydrogenase | | |
| Cow’s | [K].ALGGEDVR.[V] | 11 | Alpha-2-HS-gly | Bos taurus | 6 |
| Milk | | | coprotein | | |
| Protein | | | | | |
| Cow’s | [K].TPIVGQPSIPGGPVR.[L] | 1 | Alpha-2-HS-gly | Bos taurus | 1 |
| Milk | | | coprotein | | |
| Protein | | | | | |
| CMP or | [K].HTLNQIDSVK.[V] | 2 | Alpha-2-HS-gly | Bos taurus | 3 |
| Egg | | | coprotein | and Ovis aries | |
| CMP or | [R].GYKHTLNQIDSVK.[V] | 2 | Alpha-2-HS-gly | Bos taurus | 6 |
| Egg | | | coprotein | and Ovis aries | |
| Cow’s | [K].SPPFFEDLTLDLQPPK.[S] | 4 | Cytoplasmic | Bos taurus | 6 |
| Milk | | | aconitate hydratase | | |
| Protein | | | | | |
| Cow’s | [R].ADSLKKNQDLEFER.[N] | 4 | Cytoplasmic | Bos taurus | 6 |
| Milk | | | aconitate hydratase | | |
| Protein | | | | | |
| Cow’s | [K].TVDNFVALATGEKGFGYKDS | 3 | Peptidyl-prolyl | Bos taurus | 5 |
| Milk | K.[F] | | cis-trans | | |
| Protein | | | isomerase B | | |
| Cow’s | [K].GFGYKDSK.[F] | 1 | Peptidyl-prolyl | Bos taurus | 6 |
| Milk | | | cis-trans | | |
| Protein | | | isomerase B | | |
| Cow’s | [R].VYVVDVATEPR.[A] | 2 | Selenium-binding | Bos taurus | 6 |
| Milk | | | protein 1 | | |
| Protein | | | | | |
| Cow’s | [R].LVGQIFLGGSIVK.[G] | 1 | Selenium-binding | Bos taurus | 4 |
| Milk | | | protein 1 | | |
| Protein | | | | | |
| Cow’s | [K].LSISETYDLK.[S] | 1 | Alpha-1- | Bos taurus | 4 |
| Milk | | | antiproteinase | | |
| Protein | | | | | |
| CMP or | [K].AALTIDEK.[G] | 1 | Alpha-1- | Bos taurus | 6 |
| Egg | | | antiproteinase | and Ovis aries | |
| CMP or | [K].SVLGDVGITEVFSDR.[A] | 1 | Alpha-1- | Bos taurus | 5 |
| Egg | | | antiproteinase | and Ovis aries | |
| Cow’s | [K].QIPLTCIVDK.[R] | 6 | Metalloendopeptidase | Bos taurus | 6 |
| Milk | | | OMA1, mitochondrial | | |
| Protein | | | | | |
| Cow’s | [K].VTISCSGGR.[S] | 5 | Complement | Bos taurus | 6 |
| Milk | | | component C7 | | |
| Protein | | | | | |
| Cow’s | [R].FLEDYFDGNLKR.[Y] | 3 | Protein disulfide- | Bos taurus | 6 |
| Milk | | | isomerase A3 | | |
| Protein | | | | | |
| Cow’s | [R].AIQAAFFYLEPR.[H] | 2 | Alpha-1-acid | Bos taurus | 6 |
| Milk | | | glycoprotein | | |
| Protein | | | | | |
| Cow’s | [K].YVRPGGGFTPNFQLFEKGD | 2 | Glutathione | Bos taurus | 6 |
| Milk | VNGEKEQK.[F] | | peroxidase 3 | | |
| Protein | | | | | |
| Cow’s | [K].YHALYINALQK.[L] | 2 | Diacylglycerol O- | Bos taurus | 6 |
| Milk | | | acyltransferase | | |
| Protein | | | 2-like protein 6 | | |
| Cow’s | [K].TPTLEKQGK.[K] | 2 | Synaptonemal | Bos taurus | 6 |
| Milk | | | complex | | |
| Protein | | | protein 3 | | |
| Cow’s | [K].LAVPIILR.[V] | 1 | Alpha-fetoprotein | Bos taurus | 1 |
| Milk | | | | | |
| Protein | | | | | |
| Cow’s | [K].VLSGSIEKAK[Q] | 1 | DNA excision | Bos taurus | 6 |
| Milk | | | repair protein | | |
| Protein | | | ERCC-6-like 2 | | |
| Cow’s | [R].TVSISPTK.[K] | 1 | Fibrous sheath | Bos taurus | 6 |
| Milk | | | CABYR- | | |
| Protein | | | binding protein | | |
| Cow’s | [K].LVEFPLVAAWYQR.[I] | 1 | Glutathione S- | Bos taurus | 2 |
| Milk | | | transferase C- | | |
| Protein | | | terminal domain- | | |
| | | | containing protein | | |
| Cow’s | [K].LQHFFIGNR.[K] | 1 | Inositol 1,4,5- | Bos taurus | |
| Milk | | | trisphosphate | | |
| Protein | | | receptor- | | |
| | | | interacting protein | | |
| Cow’s | [R].EVSNKIVGYLDEEGVLDQNR | 1 | Lactoperoxidase | Bos taurus | 6 |
| Milk | [S] | | | | |
| Protein | | | | | |
| Cow’s | [R].ATDLVPR.[I] | 1 | Peroxisomal | Bos taurus | 6 |
| Milk | | | membrane | | |
| Protein | | | protein 11A | | |
| Cow’s | [R].FEILPTR.[S] | 1 | Protein-glutamine | Bos taurus | 5 |
| Milk | | | gamma-glutamyl- | | |
| Protein | | | transferase E | | |
| CMP or | [R].GSPAANVGVK.[V] | 2 | Transthyretin | Bos taurus | 6 |
| Egg | | | | and Ovis aries | |
| CMP or | [K].SSELVSANR.[L] | 1 | Antithrombin-III | Bos taurus | 5 |
| Egg | | | | and Ovis aries | |
| Cow’s | [K].LLSTLCSADVCQCAEGK.[C] | 1 | Complement C4 | Bos taurus | 1 |
| Milk | | | | and Rattus | |
| Protein | | | | norvegicus | |
| CMP or | [K].ECHLAQVPSHAVVAR.[S] | 1 | Lactotransferrin | Bos taurus | 6 |
| Egg | | | | and Ovis aries | |
| Rice | [R].WCAVAGGRLDSGK[Q] | 2 | 17 kDa alpha- | Oryza sativa | 1 |
| | | | amylase/trypsin | subsp. | |
| | | | inhibitor 2 | Japonica | |
| Rice | [K].LFNIIEPDVAVFGK.[K] | 1 | Pantoate-beta- | Oryza sativa | 1 |
| | | | alanine ligase | subsp. | |
| | | | | Japonica | |
| Rice | [K].TIEVDNTDAEGR.[L] | 4 | Proline | Oryza sativa | 6 |
| | | | aminopeptidase | subsp. | |
| | | | 1/Leucyl | Japonica and | |
| | | | aminopeptidase | Gloeobacter | |
| | | | | violaceus | |
| TBD | [R].HPGWQGTLK.[A] | 17 | | various | 6 |
| | | | | nonhuman | |
| | | | | organisms | |
| TBD | [K].GLTSLLR.[S] | 9 | | various | 6 |
| | | | | nonhuman | |
| | | | | organisms | |
| TBD | [K].AMIAYWTNFAR.[T] | 4 | | various | 6 |
| | | | | nonhuman | |
| | | | | organisms | |
| TBD | [K].AMIAYWTNFAR.[T] | 3 | | various | 6 |
| | | | | nonhuman | |
| | | | | organisms | |
| TBD | [K].EWLEEGTIAFKNWVK.[T] | 2 | | various | 3 |
| | | | | nonhuman | |
| | | | | organisms | |
| TBD | [R].LGGAEIAR.[T] | 2 | | various | 6 |
| | | | | nonhuman | |
| | | | | organisms | |
| TBD | [-].KIFERCELAR.[T] | 1 | | various | 6 |
| | | | | nonhuman | |
| | | | | organisms | |
| TBD | [K].FESNFNTQATNR.[N] | 1 | | various | 3 |
| | | | | nonhuman | |
| | | | | organisms | |
| TBD | [K].KEWLEEGTIAFK.[N] | 1 | | various | 1 |
| | | | | nonhuman | |
| | | | | organisms | |
| TBD | [R].ADLSGITK.[E] | 1 | | various | 6 |
| | | | | nonhuman | |
| | | | | organisms | |
| TBD | [R].IEYDPNR.[S] | 1 | | various | 5 |
| | | | | nonhuman | |
| | | | | organisms | |
| TBD | [R].LDSPATPERIR.[N] | 1 | | various | 4 |
| | | | | nonhuman | |
| | | | | organisms | |
| TBD | [R].NTDGSTDYGILQINSR.[W] | 1 | | various | 4 |
| | | | | nonhuman | |
| | | | | organisms | |
| TBD | [R].SALFAQINQGESITHALK.[H] | 1 | | various | 6 |
| | | | | nonhuman | |
| | | | | organisms | |
| TBD | [R].YEVPLETPR.[V] | 1 | | various | 5 |
| | | | | nonhuman | |
| | | | | organisms | |

In certain embodiments, the binding site of the probe may be initially inactive. In this condition, the binding site may be activated before bringing the probe in contact with the target molecule. Activation may include physical, chemical or optical activation. Physical activation may comprise heating the probe to activate the binding site prior to contact with target molecule. Chemical activation may include a chemical reaction to activate (e.g., open the polymer chain) prior to contact with the target molecule. Optical activation may include activation with, for example, UV rays or the like which activate the binding site to engage a target molecule.

In certain embodiments, a substrate may comprise a plurality of active site. Each detection site may be configured to detect presence of a target molecule. The target molecule may be a protein, a partially digested protein, an epitope or a molecular fraction of a larger molecule that was subjected to human digestion. In an implementation, the detection sites may be configured to exclusively detect the presence of the target molecule. In another implementation, the detection sites may be configured to detect presence of the target molecule without detecting presence of a host protein, a partially digested host protein or other molecular structure commonly present in the host. The target molecule may comprise an allergen, an enzyme, a partially digest enzyme, a protein, an amino acid, or a biopolymer.

In still another exemplary embodiment, the plurality of detection sites may be configured to detect the presence of fragments of DNA, RNA, or amino acids which are specific to the target molecule. In another embodiment, the plurality of detection sites may be configured to detect the presence of fragments of a DNA, RNA, or amino acids which may be specific to the target molecule and do not occur naturally nor are expectedly present in the host. In one embodiment, each of the plurality of detection sites may be configured to detect the presence of the same fragment of a target molecule. In some embodiments, each of the plurality of target sites may be configured to detect the presence of a different fragment of the same target molecule. The different fragments may be specifically selected from a portions of the target molecule to produce a signature specific and unique to the target molecule.

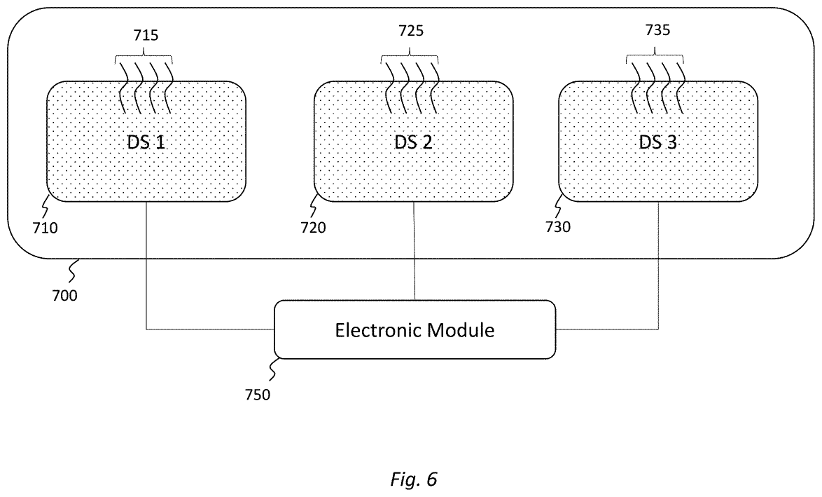

FIG. 6 is a schematic illustration of a detection substrate according to one embodiment of the disclosure. Referring to FIG. 6, substrate 600 includes detection sites 610, 620 and 630. Each of detection sites 610, 620 and 630 includes a probes 615, 625 and 635, respectively. While each detection site is shown with a plurality of probes, the disclose principles are not limited thereto and a detection site may include one probe.

In certain embodiments, the probes are configured to detect the presence of a desired target molecule. The target molecule may be an allergen. In some embodiments, the target molecule may be a substance not commonly available in the host. For example, referring to human milk, the target may be a molecule not commonly present in human milk. The target molecule may cause allergic reaction in a baby who consumes the milk. Exemplary and common target molecules include cow's milk protein, Egg, rice and various peanut. The protein of the target molecule may be partially digested in the mother's milk.

Referring again to FIG. 6, an embodiment of the disclosure uses probes 615, 625 and 635 that are configured to bind to the partially digested target molecule. That is, probes 615, 625 and 635 may be configured to attract and/or couple to the target molecule or a portion of the target molecule. In an exemplary embodiment, probes 615, 625 and 635 may comprise active sites configured to specifically target portions of the target molecule that does not react with human milk or is not digested by the human. In this manner, a principle of the disclosure detects the presence of only non-human proteins.

In some embodiments of the disclosure, detection of the target molecule may comprise detecting different fragments of the target molecule. The fragments of the target molecule may comprise one or more fragments that are not digested, reaction or otherwise changed in the host (i.e., human body) during digestion. Each fragments may comprise a unique portion of the target molecule such that a combination of the fragment may define a molecular signature of the target molecule. Referring to FIG. 6, for example, each of probes 615, 625 and 635 may be configured to detect the presence of a unique fragment of the larger target molecule. In some embodiments, each fragment may be unique and distinct from the other fragments. In other embodiments, the fragments may have some overlap. Notably, the fragments (whether overlapping or non-overlapping) may be selected such that when their present is detected as detection sites 610, 620 and 630, the presence of the target molecule in the host environment may be discerned with a reasonable certainty. Thus, detection at substrate 600 would be mutually exclusive of the proteins that exist in the host.

In some embodiments, detection of some (but not all) of the target molecule's signature may be correlated with the probability of target molecule's presence. For example, assume that each of the detection sites 610, 620 and 630 is configured to detect presence of one of three molecular signatures associated with a target molecule (e.g., peanut). Further assume that each of probes 615 and 625 communicate presence of their respective target molecule signature but no such indication is made at detection site 635. This may lead to a conclusion that peanut exists in the host's milk with a probability of 66%.

As stated, presence of a target molecule may be indicated by the presence of its undigested fragments. Detecting a compilation of undigested fragments may provide sufficient molecular signature to conclude the presence of the target molecule. The fragments may comprise biopolymers having a chain of, for example, 2-10 amino acids, 10-20 amino acids, 20-50 amino acids, 50-100 amino acids, 100-150 amino acids or 150-300 amino acids. In one embodiment, the fragments may comprise biopolymers of 2-1000, 2-500, 2-250, 2-100 and 2-50 amino acids in length.

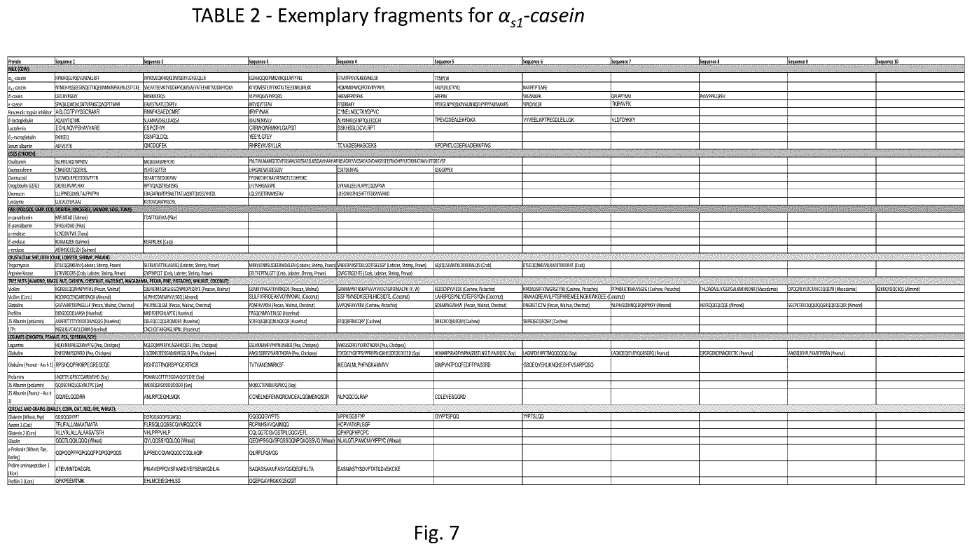

FIG. 7 schematically illustrates a circuitry for indicating presence of a target molecule according to one embodiment of the disclosure. In FIG. 7, substrate 700 comprises detection sites (DS) 710, 720 and 730. Each of detection site 710, 720 and 730 is configured with one of respective probes 715, 725 and 735. The probes 715, 725 and 735 may be configured to detect different fragments of the same target molecule according to the disclosed principles. Electronic module 750 can be configured to communicate with each of detection sites 710, 720 and 730 independently and register if and when a target molecule (or a fragment thereof) is detected as any of the detection sites 710, 720 and 730.

The electronic module may comprise one or more microprocessor circuitries and memory circuits to detect presence activation of detection sites 710, 720 and 730 when respective target molecules (or fragments thereof) binds to each site.

Furthermore, software in the form of specialized application or website can be used in combination with the substrates of the present disclosure, or with test results from the substrates, to give users additional information or diagnostic abilities. For example, software may be provided which functions in conjunction with symptom data, such as, rash, eczema, colic, diarrhea, bloody stool, mucous in stool, constipation, anaphylaxis, gas, hives, or swelling, which a user may input into the software. The software may use algorithms which may combine information from the test results and user inputs to determine which substance may be causing adverse reactions for the user. The software may additionally be configured with input fields for dates, times, and other details of contact with or consumption of various substances or potential allergens or contaminants to determine which substances were causing adverse reactions for the user. The software may optionally provide reports of various analysis, diagnoses, or database of consumption habits of the user. Further still, the software may interface with medical professionals or medical software, including electronic charts or medical diagnostic software, to provide medical and healthcare services to the user, including better diagnosis of potential allergies.

### EXAMPLES

Human milk sample analysis by mass spectrometry or amino acid sequencing yields both human and non-human derived proteins and peptides. Non-human protein and peptide expression in human milk largely originates from the maternal diet. To detect allergenic food proteins in human milk with high sensitivity and low cross-reactivity, food peptide sequences were compared to both human and non-human amino acid sequencing from human milk. Non-human peptide sequences were analyzed to determine their originating whole protein. These sequences vary in length and master protein position due to maternal digestion fluidity. Human protein digestion is dissimilar to other denaturing affects, such as heat or chemical treatment, due to the many factors involved in transferring a dietary protein from mouth to breast tissue through the digestive and circulatory system.

Many, but not all, allergenic dietary protein sequences have been discovered in human milk samples. Sequences which have been found in human milk were compared to that of similarly structured human proteins. High conservation (correlation) between non-human and human sequence alignment were discounted as possible target sequences due to possible cross reactivity and subsequent false positive results. Similarly, non-human proteins of interest without known human milk sequencing results were compared to both human milk protein sequence results as well as closely conserved human proteins to elucidate which portions of the protein may be targeted while reducing cross reactivity with human samples.

Analyzed conservation was implemented through UniProt.org protein alignment and NCBI Blastp Suite. Alignment is displayed via the following symbols denoting the degree of conservation observed in each column:

An * (asterisk) indicates positions which have a single, fully conserved residue.
A :′ (colon) indicates conservation between groups of strongly similar properties—scoring>0.5 in the Gonnet PAM 250 matrix.
A .′ (period) indicates conservation between groups of weakly similar properties—scoring=<0.5 in the Gonnet PAM 250 matrix.

FIG. 7 represents an exemplary listing of sequences relating to different allergens that may be used to identify fragments of interest. Specifically, Table 2 of FIG. 7 identifies allergens including: sequences for bovine (cow) milk, chicken eggs, fish (Pollock, Carp, Cod, Dogfish, Mackerel, Salmon, Sole, Tuna), Crustacean shellfish (Crab, Lobster, Shrimp, Prawn), Tree nuts (Almond, Brazil Nut, Cashew, Chestnut, Hazelnut, Macadamia, Pecan, Pine, Pistachio, Walnut, Coconut), Legumes (Chickpea, Peanut, Pea, Soybean/Soy), and cereal and grains (barley, corn, rice, rye and wheat). Table 2 shows cow milk with the following proteins: αₛ₁-casein, αₛ₂-casein, β-casein, κ-casein, Pancreatic trypsin inhibitor, β-lactoglobulin, Lactoferrin, β₂-microglobulin, Serum albumin. Table 2 shows eggs (chicken) with the following proteins: Ovalbumin, Ovotransferrin, Ovoglobulin G2/G3, Ovomucin and Lysozyme. Specifically, Table 1 of FIG. 8 shows the following fish proteins: α-parvalbumin, β-parvalbumin, α-enolase, β-enolase and [symbol] -enolase. Table 2 shows the following crustacean proteins: Tropomyosin and Arginine kinase. Table 2 shows the following tree nut proteins: Vicilins, Globulins, Profilins, 2S Albumin (prolamin), LTPs, Tropomyosin and Arginine kinase. Table 2 shows the following legume proteins: Legumins, Globulins, Globulins (Peanut-Ara h 1), Prolamins, 2S Albumin (prolamin) and 2S Albumin (Peanut-Ara h 2). Table 2 shows the following cereal and grain proteins: Glutenin (Wheat, Rye), Avenin 3 (Oat), Glutenin 2 (Corn), Gliadin, [symbol] -Prolamin (Wheat, Rye, Barley), Proline aminopeptidase 1 (Rice) and Profilin 3 (Corn).

As stated, an exemplary implementation of one embodiment of the disclosure relates to identifying non-human proteins in human milk to thereby identify potential allergy sources. Table 3 shows exemplary non-human protein sequences found in human milk Specifically, Table 3 shows annotated sequences in human milk, its corresponding protein name and its organism.

TABLE 3
Annotated non-human sequences in human milk.

| Source | Annotated Sequence in Human Milk | Protein Names | Organisms |
|---|---|---|---|
| Cow’s | [R].VFVQKEILDQFTEEVVKQTQR.[I] | 4- | Bos taurus |
| Milk | | trimethylaminobutyraldehyde | |
| Protein | | dehydrogenase | |
| Cow’s | [R].VFVQKEILDQFTEEWK.[Q] | 4-trimethylaminobutyraldehyde | Bos taurus |
| Milk | | dehydrogenase | |
| Protein | | | |
| Cow’s | [K].EILDQFTEEVVK.[Q] | 4-trimethylaminobutyraldehyde | Bos taurus |
| Milk | | dehydrogenase | |
| Protein | | | |
| Cow’s | [R].VIATFTCSGEKEVNLAVQDAK..[A] | 4- | Bos taurus |
| Milk | | trimethylaminobutyraldehyde | |
| Protein | | dehydrogenase | |
| Cow’s | [R].AIQAAFFYLEPR.[H] | Alpha-1-acid glycoprotein | Bos taurus |
| Milk | | | |
| Protein | | | |
| Cow’s | [K].LSISETYDLK.[S] | Alpha-1 -antiproteinase | Bos taurus |
| Milk | | | |
| Protein | | | |
| Cow’s | [K].AALTIDEK.[G] | Alpha-1 -antiproteinase | Bos taurus and |
| Milk | | | Ovis aries |
| Protein | | | |
| Cow’s | [K].SVLGDVGITEVFSDR.[A] | Alpha-1 -antiproteinase | Bos taurus and |
| Milk | | | Ovis aries |
| Protein | | | |
| Cow’s | [K].ALGGEDVR.[V] | Alpha-2-HS-glycoprotein | Bos taurus |
| Milk | | | |
| Protein | | | |
| Cow’s | [K].TPIVGQPSIPGGPVR.[L] | Alpha-2-HS-glycoprotein | Bos taurus |
| Milk | | | |
| Protein | | | |
| Cow’s | [K].HTLNQIDSVK.[V] | Alpha-2-HS-glycoprotein | Bos taurus and |
| Milk | | | Ovis aries |
| Protein | | | |
| Cow’s | [R].GYKHTLNQIDSVK.[V] | Alpha-2-HS-glycoprotein | Bos taurus and |
| Milk | | | Ovis aries |
| Protein | | | |
| Cow’s | AQFVPLPVSVSVEFAVAATDCIAK | Alpha-2-HS-glycoprotein | Bos taurus |
| Milk | | | |
| Protein | | | |
| Cow’s | [K].LAVPIILR.[V] | Alpha-feto protein | Bos taurus |
| Milk | | | |
| Protein | | | |
| Cow’s | GGVSLPEWV | Alpha-lactalbumin | Bos taurus |
| Milk | | | |
| Protein | | | |
| Cow’s | LDQWLCEKL | Alpha-lactalbumin | Bos taurus |
| Milk | | | |
| Protein | | | |
| Cow’s | NICNISCDKFLDD | Alpha-lactalbumin | Bos taurus |
| Milk | | | |
| Protein | | | |
| Cow’s | ENLLRFFVA | Alpha-S1-casein | Bos taurus |
| Milk | | | |
| Protein | | | |
| Cow’s | EKVNELSK | Alpha-S1-casein | Bos taurus |
| Milk | | | |
| Protein | | | |
| Cow’s | FFVAPFPEVFGK | Alpha-S1-casein | Bos taurus |
| Milk | | | |
| Protein | | | |
| Cow’s | HIQKEDVPSER | Alpha-S1-casein | Bos taurus |
| Milk | | | |
| Protein | | | |
| Cow’s | HQGLPQEVLNENLLR | Alpha-S1-casein | Bos taurus |
| Milk | | | |
| Protein | | | |
| Cow’s | YLGYLEQLLR | Alpha-S1-casein | Bos taurus |
| Milk | | | |
| Protein | | | |
| Cow’s | [R].FFVAPFPEVFGKEK.[V] | Alpha-S1-casein | Bos taurus |
| Milk | | | |
| Protein | | | |
| Cow’s | [K].HIQKEDVPSER.[Y] | Alpha-S1-casein | Bos taurus |
| Milk | | | |
| Protein | | | |
| Cow’s | [K].HPIKHQGLPQEVLNENLLR.[F] | Alpha-S1-casein | Bos taurus |
| Milk | | | |
| Protein | | | |
| Cow’s | [K].HQGLPQEVLNENLLR.[F] | Alpha-S1-casein | Bos taurus |
| Milk | | | |
| Protein | | | |
| Cow’s | [K].EGIHAQQKEPMIGVNQELAYFYPELFR. | Alpha-S1-casein | Bos taurus |
| Milk | [Q] | | |
| Protein | | | |
| Cow’s | [K].EKVNELSK.[D] | Alpha-S1-casein | Bos taurus |
| Milk | | | |
| Protein | | | |
| Cow’s | [R].FFVAPFPEVFGK.[E] | Alpha-S1-casein | Bos taurus |
| Milk | | | |
| Protein | | | |
| Cow’s | [R].LHSMKEGIHAQQK.[E] | Alpha-S1-casein | Bos taurus |
| Milk | | | |
| Protein | | | |
| Cow’s | [K].EGIHAQQKEPMIGVNQELAYFYPELFR. | Alpha-S1-casein | Bos taurus |
| Milk | [Q] | | |
| Protein | | | |
| Cow’s | [R].YLGYLEQLLR.[L] | Alpha-S1-casein | Bos taurus and |
| Milk | | | Ovis aries |
| Protein | | | |
| Cow’s | [K].EDVPSER.[Y] | Alpha-S1-casein | Bos taurus and |
| Milk | | | Ovis aries |
| Protein | | | |
| Cow’s | [K].TTMPLW.[-] | Alpha-S1-casein | Bos taurus and |
| Milk | | | Ovis aries |
| Protein | | | |
| Cow’s | [K].TTMPLW.[I] | Alpha-S1-casein | Bos taurus and |
| Milk | | | Ovis aries |
| Protein | | | |
| Cow’s | [K].FALPQYLK.[T] | Alpha-S2-casein | Bos taurus |
| Milk | | | |
| Protein | | | |
| Cow’s | [R].NAVPITPTLNR.[E] | Alpha-S2-casein | Bos taurus |
| Milk | | | |
| Protein | | | |
| Cow’s | [K].AMKPWIQPK.[T] | Alpha-S2-casein | Bos taurus |
| Milk | | | |
| Protein | | | |
| Cow’s | [K].TKVIPYVR.[Y] | Alpha-S2-casein | Bos taurus |
| Milk | | | |
| Protein | | | |
| Cow’s | [K].AMKPWIQPK.[T] | Alpha-S2-casein | Bos taurus |
| Milk | | | |
| Protein | | | |
| Cow’s | [K].TKVIPYVRYL.[-] | Alpha-S2-casein | Bos taurus |
| Milk | | | |
| Protein | | | |
| Cow’s | [K].ITVDDKHYQK.[A] | Alpha-S2-casein | Bos taurus and |
| Milk | | | Ovis aries |
| Protein | | | |
| Cow’s | [K].ALNEINQFYQK.[F] | Alpha-S2-casein | Bos taurus and |
| Milk | | | Ovis aries |
| Protein | | | |
| Cow’s | [K].LTEEEKNRLNFLK.[K] | Alpha-S2-casein | Bos taurus and |
| Milk | | | Ovis aries |
| Protein | | | and Sus scrota |
| Cow’s | [K].SSELVSANR.[L] | Antithrombin-III | Bos taurus and |
| Milk | | | Ovis aries |
| Protein | | | |
| Cow’s | HEQGMDQDKN | APC, WNT signalling | Bos taurus |
| Milk | | pathway regulator | |
| Protein | | | |
| Cow’s | SSLSDIDQENNNNK | APC, WNT signalling | Bos taurus |
| Milk | | pathway regulator | |
| Protein | | | |
| Cow’s | TLQIAEIKDNSGPRSNED | APC, WNT signalling | Bos taurus |
| Milk | | pathway regulator | |
| Protein | | | |
| Cow’s | VYVEQLKPTPEGDLEILLQK | Beta lactoglobulin D | Bos taurus |
| Milk | | | |
| Protein | | | |
| Cow’s | DLKLVEQQNPK | Beta-1,4- | Bos taurus |
| Milk | | galactosyltransferase 1 | |
| Protein | | | |
| Cow’s | [L].PQNIPPLTQ | Beta-casein | Bos taurus |
| Milk | | | |
| Protein | | | |
| Cow’s | QPLPPTVM | Beta-casein | Bos taurus |
| Milk | | | |
| Protein | | | |
| Cow’s | PVVVPPFLQPE.[V] | Beta-casein | Bos taurus |
| Milk | | | |
| Protein | | | |
| Cow’s | [K].AVPYPQR.[D] | Beta-casein | Bos taurus |
| Milk | | | |
| Protein | | | |
| Cow’s | [K].VKEAMAPK.[H] | Beta-casein | Bos taurus |
| Milk | | | |
| Protein | | | |
| Cow’s | [R].GPFPIIV.[-] | Beta-casein | Bos taurus |
| Milk | | | |
| Protein | | | |
| Cow’s | [K].VLPVPQKAVPYPQR.[D] | Beta-casein | Bos taurus |
| Milk | | | |
| Protein | | | |
| Cow’s | [K].VLPVPQK.[A] | Beta-casein | Bos taurus and |
| Milk | | | Ovis aries |
| Protein | | | |
| Cow’s | [K].HKEMPFPK.[Y] | Beta-casein | Bos taurus and |
| Milk | | | Ovis aries |
| Protein | | | |
| Cow’s | [K].HKEMPFPK.[Y] | Beta-casein | Bos taurus and |
| Milk | | | Ovis aries |
| Protein | | | |
| Cow’s | VALNKLK | Beta-galactosidase | Bos taurus |
| Milk | | | |
| Protein | | | |
| Cow’s | ALPMHIR | Beta-lactoglobulin | Bos taurus |
| Milk | | | |
| Protein | | | |
| Cow’s | IDALNENK | Beta-lactoglobulin | Bos taurus |
| Milk | | | |
| Protein | | | |
| Cow’s | LIVTQTMK | Beta-lactoglobulin | Bos taurus |
| Milk | | | |
| Protein | | | |
| Cow’s | LSFNPTQLEEQCHI | Beta-lactoglobulin | Bos taurus |
| Milk | | | |
| Protein | | | |
| Cow’s | TKIPAVFK | Beta-lactoglobulin | Bos taurus |
| Milk | | | |
| Protein | | | |
| Cow’s | TPEVDDEALEK | Beta-lactoglobulin | Bos taurus |
| Milk | | | |
| Protein | | | |
| Cow’s | TPEVDDEALEKFDK | Beta-lactoglobulin | Bos taurus |
| Milk | | | |
| Protein | | | |
| Cow’s | VLVLDTDYKK | Beta-lactoglobulin | Bos taurus |
| Milk | | | |
| Protein | | | |
| Cow’s | VYVEELKPTPEGDLEILLQK | Beta-lactoglobulin | Bos taurus |
| Milk | | | |
| Protein | | | |
| Cow’s | WENDECAQK | Beta-lactoglobulin | Bos taurus |
| Milk | | | |
| Protein | | | |
| Cow’s | WENDECAQKK | Beta-lactoglobulin | Bos taurus |
| Milk | | | |
| Protein | | | |
| Cow’s | SLAMAASDISLLDAQSAPLR | Beta-lactoglobulin | Bos taurus |
| Milk | | | |
| Protein | | | |
| Cow’s | [R].TPEVDDEALEKFDK.[A] | Beta-lactoglobulin | Bos taurus |
| Milk | | | |
| Protein | | | |
| Cow’s | [R]VYVEELKPTPEGDLEILLQK.[W] | Beta-lactoglobulin | Bos taurus |
| Milk | | | |
| Protein | | | |
| Cow’s | [R].TPEVDDEALEK.[F] | Beta-lactoglobulin | Bos taurus |
| Milk | | | |
| Protein | | | |
| Cow’s | [K].ALPMHIR.[L] | Beta-lactoglobulin | Bos taurus and |
| Milk | | | Ovis aries |
| Protein | | | |
| Cow’s | [K].VLVLDTDYKK.[Y] | Beta-lactoglobulin | Bos taurus and |
| Milk | | | Ovis aries |
| Protein | | | |
| Cow’s | [K].TKIPAVFK.[I] | Beta-lactoglobulin | Bos taurus and |
| Milk | | | Ovis aries |
| Protein | | | |
| Cow’s | [K].ALPMHIR.[L] | Beta-lactoglobulin | Bos taurus and |
| Milk | | | Ovis aries |
| Protein | | | |
| Cow’s | [K].LGSVYTEGGFVEGVNKK.[L] | Bile salt-activated lipase | Bos taurus |
| Milk | | (Fragment) | |
| Protein | | | |
| Cow’s | [K].RAISQSGVGLCPWAIQQDPLFWAK.[R] | Bile salt-activated lipase | Bos taurus |
| Milk | | (Fragment) | |
| Protein | | | |
| Cow’s | NVTRQAYWQIHMDQ | Cathepsin D | Bos taurus |
| Milk | | | |
| Protein | | | |
| Cow’s | NIIKSGSDEVQ | Complement C3 | Bos taurus |
| Milk | | | |
| Protein | | | |
| Cow’s | [K].LLSTLCSADVCQCAEGK.[C] | Complement C4 | Bos taurus and |
| Milk | | | Rattus |
| Protein | | | norvegicus |
| Cow’s | [K].VTISCSGGR.[S] | Complement component C7 | Bos taurus |
| Milk | | | |
| Protein | | | |
| Cow’s | [K].SPPFFEDLTLDLQPPK.[S] | Cytoplasmic aconitate | Bos taurus |
| Milk | | hydratase | |
| Protein | | | |
| Cow’s | [R].ADSLKKNQDLEFER.[N] | Cytoplasmic aconitate | Bos taurus |
| Milk | | hydratase | |
| Protein | | | |
| Cow’s | EKESLGWQK | Desmoplakin | Bos taurus |
| Milk | | | |
| Protein | | | |
| Cow’s | [K].YHALYINALQK.[L] | Diacylglycerol O- | Bos taurus |
| Milk | | acyltransferase 2-like | |
| Protein | | protein 6 | |
| Cow’s | [K].VLSGSIEKAK.[Q] | DNA excision repair protein | Bos taurus |
| Milk | | ERCC-6-like 2 | |
| Protein | | | |
| Cow’s | VNLLVDRQWQAVRNR | Ectonucleotide | Bos taurus |
| Milk | | pyrophosphatase | |
| Protein | | | |
| Cow’s | QNLAFVSMLNDIAAP | Fatty acid synthase | Bos taurus |
| Milk | | | |
| Protein | | | |
| Cow’s | [R].TVSISPTK.[K] | Fibrous sheath CABYR- | Bos taurus |
| Milk | | binding protein | |
| Protein | | | |
| Cow’s | [K].YVRPGGGFTPNFQLFEKGDVNGEKEQ | Glutathione peroxidase 3 | Bos taurus |
| Milk | K.[F] | | |
| Protein | | | |
| Cow’s | [K\|.LVEFPLVAAWYQR.[I] | Glutathione S-transferase | Bos taurus |
| Milk | | C-terminal domain- | |
| Protein | | containing protein | |
| Cow’s | NGEGQVLFETEISR | Heat shock 70 kDa protein | Bos taurus |
| Milk | | 13 | |
| Protein | | | |
| Cow’s | VSITCSGSSSNIGR | Immunoglobulin light chain | Bos taurus |
| Milk | | | |
| Protein | | | |
| Cow’s | [K].LQHFFIGNR.[K] | Inositol 1,4,5-trisphosphate | Bos taurus |
| Milk | | receptor-interacting protein | |
| Protein | | | |
| Cow’s | IKVMNDLSPKSNLR | Interferon gamma | Bos taurus |
| Milk | | | |
| Protein | | | |
| Cow’s | ESTVATLED | Kappa-casein | Bos taurus |
| Milk | | | |
| Protein | | | |
| Cow’s | IESPPEI | Kappa-casein | Bos taurus |
| Milk | | | |
| Protein | | | |
| Cow’s | SCQAQPTTMAR | Kappa-casein | Bos taurus |
| Milk | | | |
| Protein | | | |
| Cow’s | [R].SPAQILQWQVLSNTVPAK.[S] | Kappa-casein | Bos taurus |
| Milk | | | |
| Protein | | | |
| Cow’s | [R].FFSDKIAK.[Y] | Kappa-casein | Bos taurus |
| Milk | | | |
| Protein | | | |
| Cow’s | [R].YPSYGLNYYQQKPVALINNQFLPYPYY | Kappa-casein | Bos taurus |
| Milk | AKPAAVR.[S] | | |
| Protein | | | |
| Cow’s | [K].YIPIQYVLSR.[Y] | Kappa-casein | Bos taurus and |
| Milk | | | Ovis aries |
| Protein | | | |
| Cow’s | [R].EVSNKIVGYLDEEGVLDQNR.[S] | Lactoperoxidase | Bos taurus |
| Milk | | | |
| Protein | | | |
| Cow’s | NLLFNDNTECLAK | Lactotransferrin/Lactoferrin | Bos taurus |
| Milk | | | |
| Protein | | | |
| Cow’s | [K].ECHLAQVPSHAWAR.[S] | Lactotransferrin/Lactoferrin | Bos taurus and |
| Milk | | | Ovis aries |
| Protein | | | |
| Cow’s | ESPQTHYY | Lactotransferrin/Lactoferrin | Bos taurus |
| Milk | | | |
| Protein | | | |
| Cow’s | GSNFQLDQL.[Q] | Lactotransferrin/Lactoferrin | Bos taurus |
| Milk | | | |
| Protein | | | |
| Cow’s | VAWKKGSN | Lactotransferrin/Lactoferrin | Bos taurus |
| Milk | | | |
| Protein | | | |
| Cow’s | YEELGTEY | Lactotransferrin/Lactoferrin | Bos taurus |
| Milk | | | |
| Protein | | | |
| Cow’s | EKQLPNGDWPQENISGVFNKSCA | Lanosterol synthase | Bos taurus |
| Milk | | | |
| Protein | | | |
| Cow’s | KLLNNITNDLR | Macrophage scavenger | Bos taurus |
| Milk | | receptor | |
| Protein | | | |
| Cow’s | [K].QIPLTCIVDK.[R] | Metalloendopeptidase | Bos taurus |
| Milk | | OMA1, mitochondrial | |
| Protein | | | |
| Cow’s | QMERALLENE | Moesin | Bos taurus |
| Milk | | | |
| Protein | | | |
| Cow’s | IQQNSSTTEKI | Mucin-16 | Bos taurus |
| Milk | | | |
| Protein | | | |
| Cow’s | KFNITDTLMQ | Mucin-16 | Bos taurus |
| Milk | | | |
| Protein | | | |
| Cow’s | EHLYQENQYLEQENTQ | Ninein | Bos taurus |
| Milk | | | |
| Protein | | | |
| Cow’s | QEELENRTSETNTPQGNQEY | Ninein | Bos taurus |
| Milk | | | |
| Protein | | | |
| Cow’s | NKHSNLIESQENSK | Osteopontin-K, Osteopontin | Bos taurus |
| Milk | | | |
| Protein | | | |
| Cow’s | [K].AGLCQTFVYGGCR.[A] | Pancreatic trypsin inhibitor | Bos taurus |
| Milk | | | |
| Protein | | | |
| Cow’s | [R].NNFKSAEDCMR.[T] | Pancreatic trypsin inhibitor | Bos taurus |
| Milk | | | |
| Protein | | | |
| Cow’s | [R].IIRYFYNAK.[A] | Pancreatic trypsin inhibitor | Bos taurus |
| Milk | | | |
| Protein | | | |
| Cow’s | [K].TVDNFVALATGEKGFGYKDSK.[F] | Peptidyl-prolyl cistrans | Bos taurus |
| Milk | | isomerase B | |
| Protein | | | |
| Cow’s | [K].GFGYKDSK.[F] | Peptidyl-prolyl cistrans | Bos taurus |
| Milk | | isomerase B | |
| Protein | | | |
| Cow’s | [R].ATDLVPR.[I] | Peroxisomal membrane | Bos taurus |
| Milk | | protein 11A | |
| Protein | | | |
| Cow’s | [R].FLEDYFDGNLKR.[Y] | Protein disulfide-isomerase | Bos taurus |
| Milk | | A3 | |
| Protein | | | |
| Cow’s | [R].FEILPTR.[S] | Protein-glutamine gamma- | Bos taurus |
| Milk | | glutamyltransferase E | |
| Protein | | | |
| Cow’s | [R].VYVVDVATEPR.[A] | Selenium-binding protein 1 | Bos taurus |
| Milk | | | |
| Protein | | | |
| Cow’s | [R].LVGQIFLGGSIVK.[G] | Selenium-binding protein 1 | Bos taurus |
| Milk | | | |
| Protein | | | |
| Cow’s | CASFRENVLR | Serotransferrin | Bos taurus |
| Milk | | | |
| Protein | | | |
| Cow’s | AEFVEVTK | Serum albumin | Bos taurus |
| Milk | | | |
| Protein | | | |
| Cow’s | DAFLGSFLYEYSR | Serum albumin | Bos taurus |
| Milk | | | |
| Protein | | | |
| Cow’s | DLGEEHFK | Serum albumin | Bos taurus |
| Milk | | | |
| Protein | | | |
| Cow’s | DTHKSEIAHR | Serum albumin | Bos taurus |
| Milk | | | |
| Protein | | | |
| Cow’s | DVCKNYQEAK | Serum albumin | Bos taurus |
| Milk | | | |
| Protein | | | |
| Cow’s | FKDLGEEHFK | Serum albumin | Bos taurus |
| Milk | | | |
| Protein | | | |
| Cow’s | HLVDEPQNLIK | Serum albumin | Bos taurus |
| Milk | | | |
| Protein | | | |
| Cow’s | LVNELTEFAK | Serum albumin | Bos taurus |
| Milk | | | |
| Protein | | | |
| Cow’s | QNCDQFEK | Serum albumin | Bos taurus |
| Milk | | | |
| Protein | | | |
| Cow’s | RHPEYAVSVLLR | Serum albumin | Bos taurus |
| Milk | | | |
| Protein | | | |
| Cow’s | SLHTLFGDELCK | Serum albumin | Bos taurus |
| Milk | | | |
| Protein | | | |
| Cow’s | TCVADESHAGCEK | Serum albumin | Bos taurus |
| Milk | | | |
| Protein | | | |
| Cow’s | GKYLYEIAR | Serum albumin | Bos taurus |
| Milk | | | |
| Protein | | | |
| Cow’s | KQTALVELLK | Serum albumin | Bos taurus |
| Milk | | | |
| Protein | | | |
| Cow’s | [K].TPTLEKQGK.[K] | Synaptonemal complex | Bos taurus |
| Milk | | protein 3 | |
| Protein | | | |
| Cow’s | [R].GSPAANVGVK.[V] | Transthyretin | Bos taurus and |
| Milk | | | Ovis aries |
| Protein | | | |
| Cow’s | HHIELRWK | Uncharacterized protein | Bos taurus |
| Milk | | | |
| Protein | | | |
| Cow’s | QKYGVVKENVIDLTK | Uncharacterized proteins | Bos taurus |
| Milk | | | |
| Protein | | | |
| Cow’s | [R].CMLDRNEDMLITGGRHPFLAR.[Y] | Xanthine | Bos taurus |
| Milk | | dehydrogenase/oxidase | |
| Protein | | | |
| Cow’s | [R].NQPEPTVEEIEDAFQGNLCR.[C] | Xanthine | Bos taurus |
| Milk | | dehydrogenase/oxidase | |
| Protein | | | |
| Cow’s | [R].CMLDRNEDMLITGGRHPFLAR.[Y] | Xanthine | Bos taurus |
| Milk | | dehydrogenase/oxidase | |
| Protein | | | |
| Cow’s | [R].CMLDRNEDMLITGGRHPFLAR.[Y] | Xanthine | Bos taurus |
| Milk | | dehydrogenase/oxidase | |
| Protein | | | |
| Cow’s | [K].LGCGEGGCGACTVMLSKYDRLQDK.[I] | Xanthine | Bos taurus |
| Milk | | dehydrogenase/oxidase | |
| Protein | | | |
| Cow’s | NGNNPNCCMNQK | Xanthine | Bos taurus |
| Milk | | dehydrogenase/oxidase | |
| Protein | | | |
| Grain | QFKPEEMTNIIK | Profilin-3 | Zea mays |
| (Corn) | | | |
| Grain | QQQTLQQILQQQ | Alpha/beta-gliadin A-III | Triticum |
| (Wheat) | | | aestivum |
| Grain | QVLQQSSYQQLQQ | Alpha/beta-gliadin A-III | Triticum |
| (Wheat) | | | aestivum |
| Legume | RPSHQQPR | Allergen Ara h 1, Seed | Arachis hypogaea & |
| (Peanut) | | storage protein Ara h 1 | Arachis duranensis |
| Legume | QQVVELQGDRR | Allergen Ara h 2 | Arachis hypogaea |
| (Peanut) | | | |
| Legume | ANLRPCEQHLMQK | Allergen Ara h 2 | Arachis hypogaea |
| (Peanut) | | | |
| Legume | CCNELNEFENNQRCMCEALQQIMENQSD | Allergen Ara h 2 | Arachis hypogaea |
| (Peanut) | R | | |
| Legume | NLPQQCGLR | Allergen Ara h 2 | Arachis hypogaea |
| (Peanut) | | | |
| Legume | CDLEVESGGR | Allergen Ara h 2 | Arachis hypogaea |
| (Peanut) | | | |
| Rice | [R].WCAVAGGRLDSGK.[Q] | 17 kDa alpha- | Oryza sativa subsp. |
| | | amylase/trypsin inhibitor 2 | japonica |
| Rice | [K].LFNIIEPDVAVFGK.[K] | Pantoate beta-alanine | Oryza sativa subsp. |
| | | ligase | japonica |
| Rice | [K].TIEVDNTDAEGR.[L] | Proline aminopeptidase | Oryza sativa subsp. |
| | | 1/Leucyl aminopeptidase | Japonica and |
| | | | Gloeobacter |
| | | | violaceus |
| TBD | [R].HPGWQGTLK.[A] | | various nonhuman |
| | | | organisms |
| TBD | [K].GLTSLLR.[S] | | various nonhuman |
| | | | organisms |
| TBD | [K].AMIAYWTNFAR.[T) | | various nonhuman |
| | | | organisms |
| TBD | [K].AMIAYWTNFAR.[T] | | various nonhuman |
| | | | organisms |
| TBD | [K].EWLEEGTIAFKNWVK.[T] | | various nonhuman |
| | | | organisms |
| TBD | [R].LGGAEIAR.[T] | | various nonhuman |
| | | | organisms |
| TBD | [-].KIFERCELAR.[T] | | various nonhuman |
| | | | organisms |
| TBD | [K].FESNFNTQATNR.[N] | | various nonhuman |
| | | | organisms |
| TBD | [K].KEVVLEEGTIAFK.[N] | | various nonhuman |
| | | | organisms |
| TBD | [R].ADLSGITK.[E] | | various nonhuman |
| | | | organisms |
| TBD | [R].IEYDPNR.[S] | | various nonhuman |
| | | | organisms |
| TBD | [R].LDSPATPERIR.[N] | | various nonhuman |
| | | | organisms |
| TBD | [R].NTDGSTDYGILQINSR.[W] | | various nonhuman |
| | | | organisms |
| TBD | [R].SALFAQINQGESITHALK.[H] | | various nonhuman |
| | | | organisms |
| TBD | [R].YEVPLETPR.[V] | | various nonhuman |
| | | | organisms |
| TBD | EQVQELR | | various nonhuman |
| | | | organisms |
| TBD | MQDQLDQVQK | | various nonhuman |
| | | | organisms |
| TBD | KELKKVEADGEND | | various nonhuman |
| | | | organisms |
| TBD | QIANSDEVEKI | | various nonhuman |
| | | | organisms |
| TBD | KCAADESAENCDK | | various nonhuman |
| | | | organisms |
| TBD | KEPERNECFLQHK | | various nonhuman |
| | | | organisms |
| TBD | PCFSALQVDETYVPK | | various nonhuman |
| | | | organisms |
| TBD | YICENQDSISTK | | various nonhuman |
| | | | organisms |
| TBD | SALQVDETYVPK | | various nonhuman |
| | | | organisms |

FIGS. 8-59 show exemplary allergen sequences for different allergens presented in relation to Table 2 of FIG. 7. Specifically, FIGS. 8-59 show fragments of interest (highlighted in the allergen's annotated sequence) which does not overlap with the human milk. Detecting presence of the highlighted portion in an active detection site according to the disclosed principles, can help identify presence of the allergen in the human milk composition. In one embodiment, each of the plurality of detection sites associated with the substrate may include one or more of the highlighted sequence fragment.

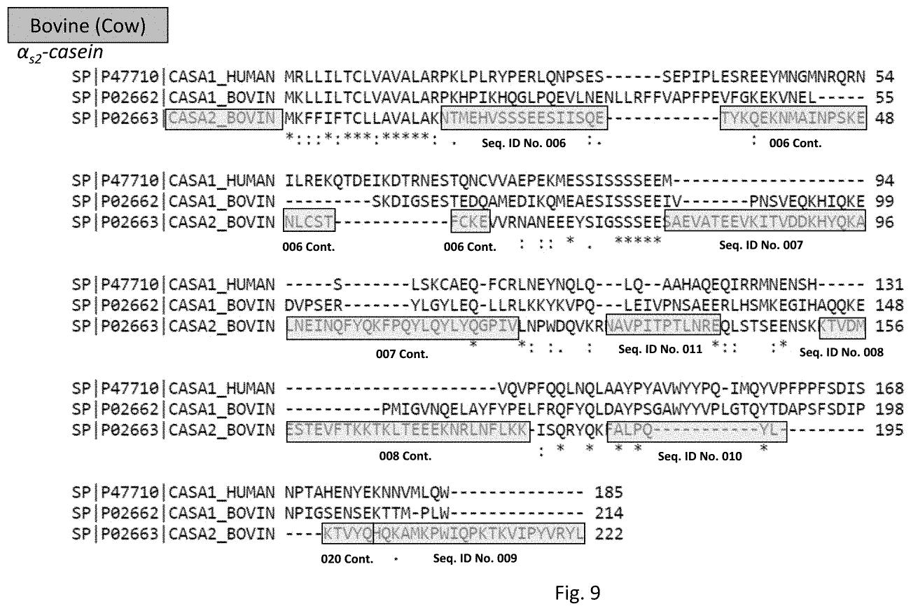

FIG. 9 shows exemplar fragments of interest for protein αₛ₁-casein which can be found in bovine milk. More specifically, FIG. 9 shows four bovine sequences along with four corresponding human sequences. Each of the highlighted portions 901 and 902 (line 55), 903 and 904 (line 106), 905, 906 (line 166) and 907 (line 214) denotes a specific fragment of the αₛ₁-casein sequence which does not overlap with human milk sequence. Each of lines 55, 96, 137 and 185 denotes the corresponding sequence from human milk Thus, a detection of any one or more of these sequences will indicate presence of αₛ₁-casein protein which may cause allergic reaction.

Referring to FIG. 9, for example, a substrate may be configured to detect presence of fragments 901, 902, 903, 904, 905, 906 and 907 in seven (7) different detection sits. On one exemplary embodiment, a substrate (600, FIG. 6) may be configured to have two or more detection sites (610, FIG. 6) to detect one or more of the fragments 901, 902, 903, 904, 905, 906 and 907.

In another embodiment, the number of detection sites may be devised to correlate the number of detectable fragments. For example, seven detection sites may be used to detect each of the seven fragments 901, 902, 903, 904, 905, 906 and 907. In still another embodiment, multiple detection sites may be used to detect presence of each of the fragments 901, 902, 903, 904, 905, 906 and 907. For example, a substrate with six detection sites may be used to detect the presence of fragments 901 and 902.

The following illustrative and non-limiting examples are provided to further represent some of the embodiments of the disclosure. These examples are used to illustrate the disclosed principles.

Example 1 relates to a method to detect presence of one or more allergen molecules in a composition of mammalian milk, the method comprising: providing a substrate having a plurality of detection sites thereon, each of the plurality of detection sites configured to detect presence of one or more allergen molecules; exposing the plurality of detection sites to a quantity of mammalian milk; detecting presence of a first allergen molecule at a first of the plurality of detection sites by detecting a fragment of DNA, RNA, or amino acids corresponding to the first allergen molecule; wherein the detected fragment excludes naturally occurring molecules present in the composition of mammalian milk.

Example 2 relates to the method of example 1, wherein at least one of the detection sites comprises a probe with an active binding site.

Example 3 relates to the method of example 2, wherein the step of detecting presence of the first allergen molecule further comprising selecting a first probe with a first active binding site to bind to the fragment of the DNA, RNA, or amino acids corresponding to the first allergen molecule.

Example 4 relates to the method of example 2, wherein the first active binding site does not bind to a naturally occurring fragment of the mammalian milk.

Example 5 relates to the method of example 1, wherein the detected fragment defines a selected portion of a DNA, RNA, or amino acid sequence associated with the allergen.

Example 6 relates to the method of example 1, wherein the composition of mammalian milk excludes the selected portion of DNA, RNA, or amino acid sequence.

Example 7 relates to the method of example 1, wherein each of the plurality of detection sites is configured to detect presence of a respective allergen molecule.

Example 8 relates to the method of example 1, wherein each of the plurality of detection sites is configured to detect presence of a different fragment of the first allergen molecule.

Example 9 relates to the method of example 1, further comprising detecting presence of a second allergen molecule at a second of the plurality of detection sites.

Example 10 relates to the method of example 1, wherein the allergen molecule is selected from the group consisting of cow's milk protein, egg, fish, crustacean, tree nut, legume, cereals, grains, or other known immune reactive groups.

Example 11 relates to the method of example 1, further comprising detecting presence of the first allergen molecule by detecting a plurality of DNA, RNA, or amino acid fragments encoding a portion of the first allergen molecule.

Example 12 relates to the method of example 1, further comprising detecting presence of a third allergen molecule at a third of the plurality of detection sites.

Example 13 relates to the method of example 1, wherein between two and ten allergen molecules are detected wherein each allergen has a detection site in the plurality of detection sites.

Example 14 relates to the method of example 1, wherein between two and five allergen molecules are detected wherein each allergen has a detection site in the plurality of detection sites.

Example 15 relates to the method of example 1, wherein a panel of at least two allergen molecules are detected, wherein each allergen has a detection site (or sites) in the plurality of detection sites.

Example 16 relates to the method of example 1, wherein a panel of at least three, four, five, six, seven, eight, nine, or ten allergen molecules are detected, wherein each allergen has its own detection site (or sites) in the plurality of detection sites.

Example 17 relates to the method of example 11, wherein a particular DNA or RNA fragment is associated with a particular allergen molecule.

Example 18 relates to the method of example 17, wherein one or more allergen has an amino acid sequence shown in any of the Figures herein or in the specification.

Example 19 relates to the method of example 17, wherein one or more allergen has an amino acid sequence shown in Figures.

Example 20 relates to the method of example 1, wherein the detection of a combination of two or more particular allergens in combination is indicative of an allergy to a known food or drink consumed by the mammal.

Example 21 relates to the method of example 11, wherein the plurality of DNA, RNA, or amino acid fragments comprise overlapping portions.

Example 22 relates to the method of example 11, wherein at least one of the plurality of DNA, RNA, or amino acid fragments is substantially different from the plurality of DNA, RNA, or amino acid fragments.

Example 23 relates to an apparatus to detect presence of one or more allergen molecules in a composition of mammalian milk, the apparatus comprising: a substrate having a plurality of detection sites thereon, each of the plurality of detection sites configured to detect presence of one or more allergen molecules when exposed to a quantity of mammalian milk; at least one detection site having a probe with an active site, the active site configured to bind to a fragment of DNA, RNA, or amino acids corresponding to a first allergen molecule to thereby detect the presence of a first allergen molecule in the quantity of mammalian milk; and wherein the detected fragment excludes naturally occurring molecules present in the composition of mammalian milk.

Example 24 relates to the apparatus of example 23, wherein at least one of the detection sites comprises a probe with an active binding site.

Example 25 relates to the apparatus of example 24, wherein the probe with an active binding site is selected to bind to the fragment of the DNA, RNA, or amino acids corresponding to the first allergen molecule.

Example 26 relates to the apparatus of example 25, wherein the first active binding site does not bind to a naturally occurring fragment of the mammalian milk.

Example 27 relates to the apparatus of example 23, the detected fragment defines a selected portion of a DNA, RNA, or amino acid sequence associated with the allergen.

Example 28 relates to the apparatus of example 23, wherein the composition of mammalian milk excludes the selected portion of DNA, RNA, or amino acid sequence.

Example 29 relates to the apparatus of example 23, wherein each of the plurality of detection sites is configured to detect presence of a respective allergen molecule.

Example 30 relates to the apparatus of example 23, wherein each of the plurality of detection sites is configured to detect presence of a different fragment of the first allergen molecule.

Example 31 relates to the apparatus of example 23, wherein one of the plurality of active sites is configured to bind to a fragment of DNA, RNA, or amino acids corresponding to a second allergen molecule to thereby detect the presence of a second allergen molecule in the quantity of mammalian milk.

Example 32 relates to the apparatus of example 23, wherein the allergen molecule is selected from the group consisting of cow's milk protein, egg, fish, crustacean, tree nut, legume, cereals, grains, or other known immune reactive groups.

Example 33 relates to the apparatus of example 23, further comprising detecting presence of the first allergen molecule by detecting a plurality of DNA, RNA, or amino acid fragments with each fragment encoding a portion of the first allergen molecule.

Example 34 relates to the apparatus of example 33, wherein the plurality of DNA, RNA, or amino acid fragments comprise overlapping portions.

Example 35 relates to the apparatus of example 33, wherein at least one of the plurality of DNA, RNA, or amino acid fragments is substantially different from the plurality of DNA, RNA, or amino acid fragments.

Except as stated above, nothing that has been stated or illustrated is intended or should be interpreted to cause a dedication of any component, step, feature, object, benefit, advantage, or equivalent to the public, regardless of whether it is or is not recited in the claims.

It should be understood that the terms and expressions used herein have the ordinary meaning as is accorded to such terms and expressions with respect to their corresponding respective areas of inquiry and study except where specific meanings have otherwise been set forth herein.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of’, and “consisting of may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method to detect presence of one or more allergen molecules in a composition of mammalian milk, the method comprising:
providing a substrate having a plurality of detection sites thereon, each of the plurality of detection sites configured to detect presence of one or more allergen molecules;
exposing the plurality of detection sites to a quantity of mammalian milk;
detecting presence of a first allergen molecule at a first of the plurality of detection sites by detecting a fragment of DNA, RNA, or amino acids corresponding to the first allergen molecule;
wherein the detected fragment excludes naturally occurring molecules present in the composition of mammalian milk.

2. The method of claim 1, wherein at least one of the detection sites comprises a probe with an active binding site.

3. The method of claim 2, wherein the step of detecting presence of the first allergen molecule further comprises selecting a first probe with a first active binding site to bind to the fragment of the DNA, RNA, or amino acids corresponding to the first allergen molecule.

4. The method of claim 3, wherein the first active binding site does not bind to a naturally occurring fragment of the mammalian milk.

5. The method of claim 1, wherein the detected fragment defines a selected portion of a DNA, RNA, or amino acid sequence associated with the allergen.

6. The method of claim 1, wherein the composition of mammalian milk excludes the selected portion of DNA, RNA, or amino acid sequence.

7. The method of claim 1, wherein each of the plurality of detection sites is configured to detect presence of a respective allergen molecule.

8. The method of claim 1, wherein each of the plurality of detection sites is configured to detect presence of a different fragment of the first allergen molecule.

9. The method of claim 1, further comprising detecting presence of a second allergen molecule at a second of the plurality of detection sites.

10. The method of claim 1, wherein the allergen molecule is selected from the group consisting of cow's milk protein, egg, fish, crustacean, tree nut, legume, cereals, grains, or other known immune reactive groups.

11. The method of claim 1, further comprising detecting presence of the first allergen molecule by detecting a plurality of DNA, RNA, or amino acid fragments encoding a portion of the first allergen molecule.

12. The method of claim 1, further comprising detecting presence of a third allergen molecule at a third of the plurality of detection sites.

13. The method of claim 1, wherein between two and ten allergen molecules are detected, and wherein each allergen has a detection site in the plurality of detection sites.

14. The method of claim 1, wherein between two and five allergen molecules are detected, and wherein each allergen has a detection site in the plurality of detection sites.

15. The method of claim 1, wherein a panel of at least two allergen molecules are detected, wherein each allergen has a detection site (or sites) in the plurality of detection sites.

16. The method of claim 1, wherein a panel of at least three, four, five, six, seven, eight, nine, or ten allergen molecules are detected, wherein each allergen has its own detection site (or sites) in the plurality of detection sites.

17. The method of claim 11, wherein a particular DNA or RNA fragment is associated with a particular allergen molecule.

18. The method of claim 17, wherein one or more allergens has an amino acid sequence shown in any of the Figures herein or in the specification.

19. The method of claim 17, wherein one or more allergens has an amino acid sequence shown in Figures.

20. The method of claim 1, wherein the detection of a combination of two or more particular allergens in combination is indicative of an allergy to a known food or drink consumed by the mammal.

21. The method of claim 11, wherein the plurality of DNA, RNA, or amino acid fragments comprise overlapping portions.

22. The method of claim 11, wherein at least one of the plurality of DNA, RNA, or amino acid fragments is substantially different from the plurality of DNA, RNA, or amino acid fragments.

23. An apparatus to detect presence of one or more allergen molecules in a composition of mammalian milk, the apparatus comprising:
a substrate having a plurality of detection sites thereon, each of the plurality of detection sites configured to detect presence of one or more allergen molecules when exposed to a quantity of mammalian milk;
at least one detection site having a probe with an active site, the active site configured to bind to a fragment of DNA, RNA, or amino acids corresponding to a first allergen molecule to thereby detect the presence of a first allergen molecule in the quantity of mammalian milk; and
wherein the detected fragment excludes naturally occurring molecules present in the composition of mammalian milk.

24. The apparatus of claim 23, wherein at least one of the detection sites comprises a probe with an active binding site.

25. The apparatus of claim 24, wherein the probe with an active binding site is selected to bind to the fragment of the DNA, RNA, or amino acids corresponding to the first allergen molecule.

26. The apparatus of claim 25, wherein the first active binding site does not bind to a naturally occurring fragment of the mammalian milk.

27. The apparatus of claim 23, wherein the detected fragment defines a selected portion of a DNA, RNA, or amino acid sequence associated with the allergen.

28. The apparatus of claim 23, wherein the composition of mammalian milk excludes the selected portion of DNA, RNA, or amino acid sequence.

29. The apparatus of claim 23, wherein each of the plurality of detection sites is configured to detect presence of a respective allergen molecule.

30. The apparatus of claim 23, wherein each of the plurality of detection sites is configured to detect presence of a different fragment of the first allergen molecule.

31. The apparatus of claim 23, wherein one of the plurality of active sites is configured to bind to a fragment of DNA, RNA, or amino acids corresponding to a second allergen molecule to thereby detect the presence of a second allergen molecule in the quantity of mammalian milk.

32. The apparatus of claim 23, wherein the allergen molecule is selected from the group consisting of cow's milk protein, egg, fish, crustacean, tree nut, legume, cereals, grains, or other known immune reactive groups.

33. The apparatus of claim 23, further comprising detecting presence of the first allergen molecule by detecting a plurality of DNA, RNA, or amino acid fragments with each fragment encoding a portion of the first allergen molecule.

34. The apparatus of claim 33, wherein the plurality of DNA, RNA, or amino acid fragments comprise overlapping portions.

35. The apparatus of claim 33, wherein at least one of the plurality of DNA, RNA, or amino acid fragments is substantially different from the plurality of DNA, RNA, or amino acid fragments.


SEQUENCE LISTING (590 sequences)

SEQ ID NO: 1 - protein, 21 aa, Bos taurus
HPIKHQGLPQEVLNENLLRFF

SEQ ID NO: 2 - protein, 30 aa, Bos taurus
IVPNSVEQKHIQKEDVPSERYLGYLEQLLR

SEQ ID NO: 3 - protein, 25 aa, Bos taurus
EGIHAQQKEPMIGVNQELAYFYPEL

SEQ ID NO: 4 - protein, 20 aa, Bos taurus
FFVAPFPEVFGKEKVNELSK

SEQ ID NO: 5 - protein, 6 aa, Bos taurus
TTMPLW

SEQ ID NO: 6 - protein, 41 aa, Bos taurus
NTMEHVSSSEESIISQETYKQEKNMAINPSKENLCSTFCKE

SEQ ID NO: 7 - protein, 45 aa, Bos taurus
SAEVATEEVKITVDDKHYQKALNEINQFYQKFPQYLQYLYQGPIV

SEQ ID NO: 8 - protein, 30 aa, Bos taurus
KTVDMESTEVFTKKTKLTEEEKNRLNFLKK

SEQ ID NO: 9 - protein, 22 aa, Bos taurus
HQKAMKPWIQPKTKVIPYVRYL

SEQ ID NO: 10 - protein, 12 aa, Bos taurus
FALPQYLKTVYQ

SEQ ID NO: 11 - protein, 12 aa, Bos taurus
NAVPITPTLNRE

SEQ ID NO: 12 - protein, 11 aa, Bos taurus
LEELNVPGEIV

SEQ ID NO: 13 - protein, 11 aa, Bos taurus
RINKKIEKFQS

SEQ ID NO: 14 - protein, 15 aa, Bos taurus
VLPVPQKAVPYPQRD

SEQ ID NO: 15 - protein, 12 aa, Bos taurus
HKEMPFPKYPVE

SEQ ID NO: 16 - protein, 7 aa, Bos taurus
GPFPIIV

SEQ ID NO: 17 - protein, 8 aa, Bos taurus
VKEAMAPK

SEQ ID NO: 18 - protein, 8 aa, Bos taurus
QPLPPTWM

SEQ ID NO: 19 - protein, 12 aa, Bos taurus
PVVVPPFLQPEV

SEQ ID NO: 20 - protein, 29 aa, Bos taurus
SPAQILQWQVLSNTVPAKSCQAQPTTMAR

SEQ ID NO: 21 - protein, 16 aa, Bos taurus
EAVESTVATLEDSPEV

SEQ ID NO: 22 - protein, 11 aa, Bos taurus
INTVQVTSTAV

SEQ ID NO: 23 - protein, 9 aa, Bos taurus
FFSDKIAKY

SEQ ID NO: 24 - protein, 10 aa, Bos taurus
YIPIQYVLSR

SEQ ID NO: 25 - protein, 35 aa, Bos taurus
YPSYGLNYYQQKPVALINNQFLPYPYYAKPAAVRS

SEQ ID NO: 26 - protein, 8 aa, Bos taurus
TKIPAVFK

SEQ ID NO: 27 - protein, 16 aa, Bos taurus
AGLCQTFVYGGCRAKR

SEQ ID NO: 28 - protein, 13 aa, Bos taurus
RNNFKSAEDCMRT

SEQ ID NO: 29 - protein, 9 aa, Bos taurus
IIRYFYNAK

SEQ ID NO: 30 - skipped

SEQ ID NO: 31 - protein, 11 aa, Bos taurus
AQALIVTQTMK

SEQ ID NO: 32 - protein, 20 aa, Bos taurus
SLAMAASDISLLDAQSAPLR

SEQ ID NO: 33 - protein, 11 aa, Bos taurus
IDALNENKVLV

SEQ ID NO: 34 - protein, 21 aa, Bos taurus
ALPMHIRLSFNPTQLEEQCHI

SEQ ID NO: 35 - protein, 15 aa, Bos taurus
TPEVDDEALEKFDKA

SEQ ID NO: 36 - protein, 20 aa, Bos taurus
VYVEELKPTPEGDLEILLQK

SEQ ID NO: 37 - protein, 9 aa, Bos taurus
VLDTDYKKY

SEQ ID NO: 38 - protein, 16 aa, Bos taurus
ECHLAQVPSHAVVARS

SEQ ID NO: 39 - protein, 8 aa, Bos taurus
ESPQTHYY

SEQ ID NO: 40 - protein, 17 aa, Bos taurus
CRRWQWRMKKLGAPSIT

SEQ ID NO: 41 - protein, 14 aa, Bos taurus
SSKHSSLDCVLRPT

SEQ ID NO: 42 - protein, 7 aa, Bos taurus
EKIKSEQ

SEQ ID NO: 43 - protein, 10 aa, Bos taurus
GSNFQLDQLQ

SEQ ID NO: 44 - protein, 10 aa, Bos taurus
YEEYLGTEYV

SEQ ID NO: 45 - protein, 8 aa, Bos taurus
AEFVEVTK

SEQ ID NO: 46 - protein, 8 aa, Bos taurus
QNCDQFEK

SEQ ID NO: 47 - protein, 12 aa, Bos taurus
RHPEYAVSVLLR

SEQ ID NO: 48 - protein, 14 aa, Bos taurus
TCVADESHAGCEKS

SEQ ID NO: 49 - protein, 20 aa, Bos taurus
KPDPNTLCDEFKADEKKFWG

SEQ ID NO: 50 - protein, 16 aa, Gallus gallus
SSLRDILNQITKPNDV

SEQ ID NO: 51 - protein, 14 aa, Gallus gallus
MGSIGAASMEFCFD

SEQ ID NO: 52 - protein, 82 aa, Gallus gallus
YNLTSVLMAMGITDVFSSSANLSGISSAESLKISQAVHAAHAEINEAGREVVGSAEAGVDAASVSEEFRADHPFLFCIKHIA

SEQ ID NO: 53 - protein, 15 aa, Gallus gallus
CNNLRDLTQQERISL

SEQ ID NO: 54 - protein, 11 aa, Gallus gallus
YEHTEGSTTSY

SEQ ID NO: 55 - protein, 16 aa, Gallus gallus
LHRGAIEWEGIESGSV

SEQ ID NO: 56 - protein, 10 aa, Gallus gallus
CSKTDERPAS

SEQ ID NO: 57 - protein, 9 aa, Gallus gallus
GSGGIPPEK

SEQ ID NO: 58 - protein, 20 aa, Gallus gallus
LVCNKDLRPICGTDGVTYTN

SEQ ID NO: 59 - protein, 15 aa, Gallus gallus
SSYANTTSEDGKVMV

SEQ ID NO: 60 - protein, 27 aa, Gallus gallus
TYGNKCNFCNAVVESNGTLTLSHFGKC

SEQ ID NO: 61 - protein, 14 aa, Gallus gallus
GIEVELRVAPLHAV

SEQ ID NO: 62 - protein, 16 aa, Gallus gallus
RPTVQAQSTREAESKS

SEQ ID NO: 63 - protein, 13 aa, Gallus gallus
LFLTVHIGAGSPD

SEQ ID NO: 64 - protein, 22 aa, Gallus gallus
LVRAALLEELFLAPVCQQVPAW

SEQ ID NO: 65 - protein, 21 aa, Gallus gallus
LLLFPNEQLMSLTALFPVTPN

SEQ ID NO: 66 - protein, 34 aa, Gallus gallus
ERAGAFNMTIPSMLTTATLAQKITQVGSLYHEDL

SEQ ID NO: 67 - protein, 16 aa, Gallus gallus
LQLSVSDTRMMISTAV

SEQ ID NO: 68 - protein, 25 aa, Gallus gallus
LREGVHLPHLSHFTYTDISVVVHKD

SEQ ID NO: 69 - protein, 13 aa, Gallus gallus
LILVLCFLPLAAL

SEQ ID NO: 70 - protein, 14 aa, Gallus gallus
KGTDVQAWIRGCRL

SEQ ID NO: 71 - protein, 8 aa, Salmo salar
KAFLNEAD

SEQ ID NO: 72 - protein, 11 aa, Esox lucius
TDAETKAFLKA

SEQ ID NO: 73 - protein, 9 aa, Esox lucius
SFAGLKDAD

SEQ ID NO: 74 - protein, 10 aa, Thunnus albacares
LCNQDVTVIE

SEQ ID NO: 75 - protein, 10 aa, Salmo salar
KDIAAKLIEK

SEQ ID NO: 76 - protein, 10 aa, Cyprinus carpio
KEIAPKLIEK

SEQ ID NO: 77 - protein, 15 aa, Salmo salar
PDAERHISGEELSDI

SEQ ID NO: 78 - protein, 11 aa, Unknown
  Present in Penaeus aztecus, Penaeus monodon and Homarus americanus
DTLEQQNKEAN

SEQ ID NO: 79 - protein, 17 aa, Unknown
  Present in Penaeus aztecus, Penaeus monodon and Homarus americanus
SEERLNTATTKLAEASQ

SEQ ID NO: 80 - protein, 21 aa, Unknown
  Present in Penaeus aztecus, Penaeus monodon and Homarus americanus
MRKVLENRSLSDEERMDALEN

SEQ ID NO: 81 - protein, 24 aa, Unknown
  Present in Penaeus aztecus, Penaeus monodon and Homarus americanus
VNEKEKYKSITDELDQTFSELSGY

SEQ ID NO: 82 - protein, 21 aa, Charybdis feriata
AQEQLSAANTKLDEKEKALQN

SEQ ID NO: 83 - protein, 24 aa, Charybdis feriata
DTLEQQNKEANLRAEKTEEEIRAT

SEQ ID NO: 84 - protein, 10 aa, Unknown
  Present in Penaeus vannamei, Limulus polyphemus, Penaeus monodon and Homarus gammarus
ISTRVRCGRS

SEQ ID NO: 85 - protein, 17 aa, Unknown
  Present in Carya illinoinensis and Juglans regia
RGREEEQQRHNPYYFHS

SEQ ID NO: 86 - protein, 25 aa, Unknown
  Present in Carya illinoinensis and Juglans regia
QEERERRRGRGEGGQNPRDPEQRYE

SEQ ID NO: 87 - protein, 19 aa, Unknown
  Present in Carya illinoinensis and Juglans regia
GDVIRVPAGATEYVINQDS

SEQ ID NO: 88 - protein, 13 aa, Unknown
  Present in Anacardium occidentale and Pistacia vera
EEEDENPYVFEDE

SEQ ID NO: 89 - protein, 20 aa, Unknown
  Present in Anacardium occidentale and Pistacia vera
HMDADSIFFVSWGRGTITKI

SEQ ID NO: 90 - protein, 18 aa, Unknown
  Present in Anacardium occidentale and Pistacia vera
PFYNSRATKIAIVVSGEG

SEQ ID NO: 91 - protein, 26 aa, Macadamia integrifolia
THLDADAILLVIGGRGALKMIHRDNR

SEQ ID NO: 92 - protein, 20 aa, Macadamia integrifolia
DPQQREYEDCRRHCEQQEPR

SEQ ID NO: 93 - protein, 17 aa, Prunus dulcis
KQCKRGCENQARDDVQK

SEQ ID NO: 94 - protein, 18 aa, Prunus dulcis
VLPHHCDAEAIYVVLSGQ

SEQ ID NO: 95 - protein, 21 aa, Cocos nucifera
SLILFVRRGEAKVGYIYKNKL

SEQ ID NO: 96 - protein, 23 aa, Cocos nucifera
SSFYIVNSDKSERLHIICSIDTL

SEQ ID NO: 97 - protein, 19 aa, Cocos nucifera
LAHSPGSYNLYDTEPSYQN

SEQ ID NO: 98 - protein, 32 aa, Cocos nucifera
RIVKAQREAVILPTSPHREMEEINGKKWKGEE

SEQ ID NO: 99 - protein, 17 aa, Unknown
  Present in Carya illinoinensis, Juglans nigra and Castanea sativa
GVAVVRRTIEPNGLLLP

SEQ ID NO: 100 - protein, 11 aa, Unknown
  Present in Carya illinoinensis, Juglans nigra and Castanea sativa
PVLRWLQLSAE

SEQ ID NO: 101 - protein, 10 aa, Unknown
  Present in Carya illinoinensis, Juglans nigra and Castanea sativa
PQNFAVVKRA

SEQ ID NO: 102 - protein, 12 aa, Anacardium occidentale
VVPQNFAVVKRA

SEQ ID NO: 103 - protein, 13 aa, Anacardium occidentale
GDIIAIPAGVAHW

SEQ ID NO: 104 - protein, 11 aa, Anacardium occidentale
DNGIEETICTM

SEQ ID NO: 105 - protein, 20 aa, Prunus dulcis
NLFHVSSDHNQLDQNPRKFY

SEQ ID NO: 106 - protein, 12 aa, Prunus dulcis
HEERQQEQLQQE

SEQ ID NO: 107 - protein, 26 aa, Prunus dulcis
SGCPETFEESQQSSQQGRQQEQEQER

SEQ ID NO: 108 - protein, 13 aa, Corylus avellana
DIDGQGQQLAASA

SEQ ID NO: 109 - protein, 14 aa, Corylus avellana
MKDFDEPGHLAPTG

SEQ ID NO: 110 - protein, 14 aa, Corylus avellana
TPGQCNMVVERLGD

SEQ ID NO: 111 - protein, 22 aa, Corylus avellana
AAAFRTTITTVDVDEDIVNQQG

SEQ ID NO: 112 - protein, 16 aa, Corylus avellana
QELEQCCQQLRQMDER

SEQ ID NO: 113 - protein, 17 aa, Corylus avellana
SCREQAQRQQNLNQCQR

SEQ ID NO: 114 - protein, 12 aa, Anacardium occidentale
EEQQRFRNCQRY

SEQ ID NO: 115 - protein, 12 aa, Anacardium occidentale
DRRCRCQNLEQM

SEQ ID NO: 116 - protein, 14 aa, Anacardium occidentale
SISPSQGCQFQSSY

SEQ ID NO: 117 - protein, 15 aa, Corylus avellana
MGSLKLVCAVLLCMM

SEQ ID NO: 118 - protein, 16 aa, Corylus avellana
CNCLKDTAKGIAGLNP

SEQ ID NO: 119 - protein, 18 aa, Unknown
  Present in Pisum sativum and Cicer arietinum
HQKVNRFREGDIIAVPTG

SEQ ID NO: 120 - protein, 21 aa, Unknown
  Present in Pisum sativum and Cicer arietinum
NQLDQMPRRFYLAGNHEQEFL

SEQ ID NO: 121 - protein, 20 aa, Unknown
  Present in Pisum sativum and Cicer arietinum
GSLHKNAMFVPHYNLNANSI

SEQ ID NO: 122 - protein, 20 aa, Unknown
  Present in Pisum sativum and Cicer arietinum
AAKSLSDRFSYVAFKTNDRA

SEQ ID NO: 123 - protein, 14 aa, Unknown
  Present in Pisum sativum and Cicer arietinum
ENEGNNIFSGFKRD

SEQ ID NO: 124 - protein, 22 aa, Unknown
  Present in Pisum sativum and Cicer arietinum
LQGRNEDEEKGAIVKVKGGLSI

SEQ ID NO: 125 - protein, 20 aa, Unknown
  Present in Pisum sativum and Cicer arietinum
AAKSLSDRFSYVAFKTNDRA

SEQ ID NO: 126 - protein, 34 aa, Glycine max
EDEDEEYGRTPSYPPRRPSHGKHEDDEDEDEEED

SEQ ID NO: 127 - protein, 35 aa, Glycine max
HENIARPSRADFYNPKAGRISTLNSLTLPALRQFG

SEQ ID NO: 128 - protein, 19 aa, Glycine max
LAGNPDIEHPETMQQQQQQ

SEQ ID NO: 129 - protein, 18 aa, Arachis hypogaea
LAGKQEQEFLRYQQRSGR

SEQ ID NO: 130 - protein, 18 aa, Arachis hypogaea
QPGRGDKDFNNGIEETIC

SEQ ID NO: 131 - protein, 20 aa, Arachis hypogaea
AAKSQSEHFLYVAFKTNSRA

SEQ ID NO: 132 - protein, 20 aa, Arachis hypogaea
RPSHQQPRKIRPEGREGEQE

SEQ ID NO: 133 - protein, 20 aa, Arachis hypogaea
RGHTGTTNQRSPPGERTRGR

SEQ ID NO: 134 - protein, 15 aa, Arachis hypogaea
TVTVANGNNRKSFNL

SEQ ID NO: 135 - protein, 20 aa, Arachis hypogaea
IKEGALMLPHFNSKAMVIVV

SEQ ID NO: 136 - protein, 21 aa, Arachis hypogaea
ISMPVNTPGQFEDFFPASSRD

SEQ ID NO: 137 - protein, 26 aa, Arachis hypogaea
GSGEQVEKLIKNQKESHFVSARPQSQ

SEQ ID NO: 138 - protein, 21 aa, Glycine max
LNGETYLGPSCCQAIRIVGHD

SEQ ID NO: 139 - protein, 24 aa, Glycine max
PDMIASLGFTTEEGDVLQGYCDSE

SEQ ID NO: 140 - protein, 17 aa, Glycine max
QQDSCRKQLQGVNLTPC

SEQ ID NO: 141 - protein, 17 aa, Glycine max
IMEKIQGRGDDDDDDDD

SEQ ID NO: 142 - protein, 17 aa, Glycine max
MQKCCTEMSELRSPKCQ

SEQ ID NO: 143 - protein, 10 aa, Arachis hypogaea
QQWELQGDRR

SEQ ID NO: 144 - protein, 13 aa, Arachis hypogaea
ANLRPCEQHLMQK

SEQ ID NO: 145 - protein, 29 aa, Arachis hypogaea
CCNELNEFENNQRCMCEALQQIMENQSDR

SEQ ID NO: 146 - protein, 11 aa, Arachis hypogaea
NLPQQCGLRAP

SEQ ID NO: 147 - protein, 11 aa, Arachis hypogaea
CDLEVESGGRD

SEQ ID NO: 148 - protein, 11 aa, Unknown
  Present in Triticum aestivum and Secale cereale
GQGQQGYYPTS

SEQ ID NO: 149 - protein, 14 aa, Unknown
  Present in Triticum aestivum and Secale cereale
QQPGQGQQPGQWQQ

SEQ ID NO: 150 - protein, 10 aa, Unknown
  Present in Triticum aestivum and Secale cereale
GQGQQGYYPT

SEQ ID NO: 151 - protein, 10 aa, Unknown
  Present in Triticum aestivum and Secale cereale
VPPKGGSFYP

SEQ ID NO: 152 - protein, 9 aa, Unknown
  Present in Triticum aestivum and Secale cereale
GYYPTSPQQ

SEQ ID NO: 153 - protein, 8 aa, Unknown
  Present in Triticum aestivum and Secale cereale
YYPTSLQQ

SEQ ID NO: 154 - protein, 17 aa, Avena sativa
TFLIFALLAMAATMATA

SEQ ID NO: 155 - protein, 21 aa, Avena sativa
FLRSQILQQSSCQVMRQQCCR

SEQ ID NO: 156 - protein, 16 aa, Avena sativa
RCPAIHSVVQAIIMQQ

SEQ ID NO: 157 - protein, 12 aa, Avena sativa
HCPVATAPLGGF

SEQ ID NO: 158 - protein, 19 aa, Zea mays
VLLVALALLALAASATSTH

SEQ ID NO: 159 - protein, 10 aa, Zea mays
VHLPPPVHLP

SEQ ID NO: 160 - protein, 22 aa, Zea mays
CQLQGTCGVGSTPILGQCVEFL

SEQ ID NO: 161 - protein, 11 aa, Zea mays
QPHPQPHPCPC

SEQ ID NO: 162 - protein, 12 aa, Triticum aestivum
QQQTLQQILQQQ

SEQ ID NO: 163 - protein, 13 aa, Triticum aestivum
QVLQQSSYQQLQQ

SEQ ID NO: 164 - protein, 25 aa, Triticum aestivum
QEQYPSGQVSFQSSQQNPQAQGSVQ

SEQ ID NO: 165 - protein, 19 aa, Triticum aestivum
NLALQTLPAMCNVYIPPYC

SEQ ID NO: 166 - protein, 23 aa, Unknown
  Present in Secale cereale, Hordeum brachyantherum and Triticum aestivum
QQPQQPFPQPQQQFPQPQQPQQS

SEQ ID NO: 167 - protein, 22 aa, Unknown
  Present in Secale cereale, Hordeum brachyantherum and Triticum aestivum
ILPRSDCQVMQQQCCQQLAQIP

SEQ ID NO: 168 - protein, 12 aa, Unknown
  Present in Secale cereale, Hordeum brachyantherum and Triticum aestivum
QILRPLFQIVQG

SEQ ID NO: 169 - protein, 14 aa, Oryza sativa
KTIEVNNTDAEGRL

SEQ ID NO: 170 - protein, 28 aa, Oryza sativa
PNAVEPPQVSFAAKDVEFSEWKGDILAI

SEQ ID NO: 171 - protein, 25 aa, Oryza sativa
SAQASSAAIVFASVGGIQEDFKLTA

SEQ ID NO: 172 - protein, 25 aa, Oryza sativa
EASNIASTYSDVFTATILDVEKCKE

SEQ ID NO: 173 - protein, 12 aa, Zea mays
QFKPEEMTNIIK

SEQ ID NO: 174 - protein, 14 aa, Zea mays
EHLMCEIEGHHLSS

SEQ ID NO: 175 - protein, 18 aa, Zea mays
QGEPGAVIRGKKGSGGIT

SEQ ID NO: 176 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 177 - protein, 185 aa, Homo sapiens
MRLLILTCLVAVALARPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 178 - protein, 222 aa, Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 179 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 180 - protein, 226 aa, Homo sapiens
MKVLILACLVALALARETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 181 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 182 - protein, 182 aa, Homo sapiens
MKSFLLVVNALALTLPFLAVEVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 183 - protein, 79 aa, Homo sapiens
MKVTGIFLLSALALLSLSGNTGADSLGREAKCYNELNGCTKIYDPVCGTDGNTYPNECVLCFENRKRQTSILIQKSGPC

SEQ ID NO: 184 - protein, 100 aa, Bos taurus
MKMSRLCLSVALLVLLGTLAASTPGCDTSNQAKAQRPDFCLEPPYTGPCKARIIRYFYNAKAGLCQTFVYGGCRAKRNNFKSAEDCMRTCGGAIGPWENL

SEQ ID NO: 185 - protein, 178 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 186 - protein, 180 aa, Homo sapiens
MLCLLLTLGVALVCGVPAMDIPQTKQDLELPKLAGTWHSMAMATNNISLMATLKAPLRVHITSLLPTPEDNLEIVLHRWENNSCVEKKVLGEKTENPKKFKINYTVANEATLLDTDYDNFLFLCLQDTTTPIQSMMCQYLARVLVEDDEIMQGFIRAFRPLPRHLWYLLDLKQMEEPCRF

SEQ ID NO: 187 - protein, 710 aa, Homo sapiens
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 188 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 189 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 190 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 191 - protein, 609 aa, Homo sapiens [sequence omitted]

SEQ ID NO: 192 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 193 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 194 - protein, 423 aa, Homo sapiens
MAMVVSSWRDPQDDVAGGNPGGPNPAAQAARGGGGGAGEQQQQAGSGAPHTPQTPGQPGAPATPGTAGDKGQGPPGSGQSQQHIECVVCGDKSSGKHYGQFTCEGCKSFFKRSVRRNLTYTCRANRNCPIDQHHRNQCQYCRLKKCLKVGMRREAVQRGRMPPTQPNPGQYALTNGDPLNGHCYLSGYISLLLRAEPYPTSRYGSQCMQPNNIMGIENICELAARLLFSAVEWARNIPFFPDLQITDQVSLLRLTWSELFVLNAAQCSMPLHVAPLLAAAGLHASPMSADRVVAFMDHIRIFQEQVEKLKALHVDSAEYSCLKAIVLFTSDACGLSDAAHIESLQEKSQCALEEYVRSQYPNQPSRFGKLLLRLPSLRTVSSSVIEQLFFVRLVGKTPIETLIRDMLLSGSSFNWPYMSIQCS

SEQ ID NO: 195 - protein, 705 aa, Gallus gallus
MKLILCTVLSLGIAAVCFAAPPKSVIRWCTISSPEEKKCNNLRDLTQQERISLTCVQKATYLDCIKAIANNEADAISLDGGQAFEAGLAPYKLKPIAAEVYEHTEGSTTSYYAVAVVKKGTEFTVNDLQGKTSCHTGLGRSAGWNIPIGTLLHRGAIEWEGIESGSVEQAVAKFFSASCVPGATIEQKLCRQCKGDPKTKCARNAPYSGYSGAFHCLKDGKGDVAFVKHTTVNENAPDQKDEYELLCLDGSRQPVDNYKTCNWARVAAHAVVARDDNKVEDIWSFLSKAQSDFGVDTKSDFHLFGPPGKKDPVLKDLLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISSLKTCNPSDILQMCSFLEGK

SEQ ID NO: 196 - protein, 210 aa, Gallus gallus
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 197 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 198 - protein, 458 aa, Homo sapiens
MAWASRLGLLLALLLPVVGASTPGTVVRLNKAALSYVSEIGKAPLQRALQVTVPHFLDWSGEALQPTRIRILNVHVPRLHLKFIAGFGVRLLAAANFTFKVFRAPEPLELTLPVELLADTRVTQSSIRTPVVSISACSLFSGHANEFDGSNSTSHALLVLVQKHIKAVLSNKLCLSISNLVQGVNVHLGTLIGLNPVGPESQIRYSMVSVPTVTSDYISLEVNAVLFLLGKPIILPTDATPFVLPRHVGTEGSMATVGLSQQLFDSALLLLQKAGALNLDITGQLRSDDNLLNTSALGRLIPEVARQFPEPMPVVLKVRLGATPVAMLHTNNATLRLQPFVEVLATASNSAFQSLFSLDVVVNLRLQLSVSKVKLQGTTSVLGDVQLTVASSNVGFIDTDQVRTLMGTVFEKPLLDHLNALLAMGIALPGVVNLHYVAPEIFVYEGYVVISSGLFYQS

SEQ ID NO: 199 - protein, 439 aa, Gallus gallus
MGALLALLDPVQPTRAPDCGGILTPLGLSYLAEVSKPHAEVVLRQDLMAQRASDLFLGSMEPSRNRITSVKVADLWLSVIPEAGLRLGIEVELRVAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDISVVVHKDYVLVPCKLKLRSTMA

SEQ ID NO: 200 - protein, 662 aa, Homo sapiens
PSLRTIPVVRASNPAHNGRVCSTWGSFHYKTFDGDVFRFPGLCNYVFSEHCGAAYEDFNIQLRRSQESAAPTLSRVLMKVDGVVIQLTKGSVLVNGHPVLLPFSQSGVLIQQSSSYTKVEARLGLVLMWNHDDSLLLELDTKYANKTCGLCGDFNGMPVVSELLSHNTKLTPMEFGNLQKMDDPTDQCQDPVPEPPRNCSTGFGICEELLHGQLFSGCVALVDVGSYLEACRQDLCFCEDTDLLSCVCHTLAEYSRQCTHAGGLPQDWRGPDFCPQKCPNNMQYHECRSPCADTCSNQEHSRACEDHCVAGCFCPEGTVLDDIGQTGCVPVSKCACVYNGAAYAPGATYSTDCTNCTCSGGRWSCQEVPCPGTCSVLGGAHFSTFDGKQYTVHGDCSYVLTKPCDSSAFTVLAELRRCGLTDSETCLKSVTLSLDGAQTVVVIKASGEVFLNQIYTQLPISAANVTIFRPSTFFIIAQTSLGLQLNLQLVPTMQLFMQLAPKLRGQTCGLCGNFNSIQADDFRTLSGVVEATAAAFFNTFKTQAACPNIRNSFEDPCSLSVENEKYAQHWCSQLTDADGPFGRCHAAVKPGTYYSNCMFDTCNCERSEDCLCAALSSYVHACAAKGVQLGGWRDGVCTKPMTTCPKSMTYHYHVSTCQPTCR

SEQ ID NO: 201 - protein, 147 aa, Gallus gallus
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 202 - protein, 148 aa, Homo sapiens
MKALIVLGLVLLSVTVQGKVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 203 - protein, 110 aa, Homo sapiens
MSMTDLLNAEDIKKAVGAFSATDSFDHKKFFQMVGLKKKSADDVKKVFHMLDKDKSGFIEEDELGFILKGFSPDARDLSAKETKMLMAAGDKDGDGKIGVDEFSTLVAES

SEQ ID NO: 204 - protein, 109 aa, Salmo salar
MAMNSILNAADIKKALDAFAAADSFDHKKFFEMVGLKAKSAEDVKKAFLVLDADASGFIEEEELKFVLKGFASDGRDLTDKETKAFLNEADKDGDGMIGIDEFVALVHE

SEQ ID NO: 205 - protein, 108 aa, Esox lucius
AKDLLKADDIKKALDAVKAEGSFNHKKFFALVGLKAMSANDVKKVFKAIDADASGFIEEEELKFVLKSFAADGRDLTDAETKAFLKAADKDGDGKIGIDEFETLVHEA

SEQ ID NO: 206 - protein, 107 aa, Esox lucius
SFAGLKDADVAAALAACSAADSFKHKEFFAKVGLASKSLDDVKKAFYVIDQDKSGFIEEDELKLFLQNFSPSARALTDAETKAFLADGDKDGDGMIGVDEFAAMIKA

SEQ ID NO: 207 - protein, 109 aa, Homo sapiens
MSITDVLSADDIAAALQECRDPDTFEPQKFFQTSGLSKMSANQVKDVFRFIDNDQSGYLDEEELKFFLQKFESGARELTESETKSLMAAADNDGDGKIGAEEFQEMVHS

SEQ ID NO: 208 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 209 - protein, 432 aa, Thunnus albacares
MSILKIHAREIFDSRGNPTVEVDLYTKKGLFRAAVPSGASTGIYEALELRDNDKTRYMGKGVKRAVKYINEFLAPALCNQDVTVIEQEKIDKLMLDMDGTENKYKFGANAILGVSLAVCKAGAAEKGVPLYRHIADLAGNPEVILPVPAFNVINGGSHAGNKLAMQEFMILPVGASSFKDAMRIGAEVYHNLKNVIKEKYGKDATNVGDEGGFAPNILENKEALKLLKNAIGKAGYTDKIVIGMDVAASEFYKGGKYDLDFKSPDDPSRYIPSDKLADLYKGFVKDYPVVSIEDPFDQDDWEAWSKFTASTSIQVVGDDLTVTNPKRIAKGVAEKSCNCLLLKVNQIGSVTESLQACKMAQSSGWGVMVSHRSGETEDTLISDLVVGLCTGQIKTGAPCRSERLAKYNQLMRIEEELGAKAKFAGKNFRHPI

SEQ ID NO: 210 - protein, 434 aa, Homo sapiens
MAMQKIFAREILDSRGNPTVEVDLHTAKGRFRAAVPSGASTGIYEALELRDGDKGRYLGKGVLKAVENINNTLGPALLQKKLSVVDQEKVDKFMIELDGTENKSKFGANAILGVSLAVCKAGAAEKGVPLYRHIADLAGNPDLILPVPAFNVINGGSHAGNKLAMQEFMILPVGASSFKEAMRIGAEVYHHLKGVIKAKYGKDATNVGDEGGFAPNILENNEALELLKTAIQAAGYPDKVVIGMDVAASEFYRNGKYDLDFKSPDDPARHITGEKLGELYKSFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAIFAGRKFRNPKAK

SEQ ID NO: 211 - protein, 434 aa, Salmo salar
MSITKIHAREILDSRGNPTVEVDLYTAKGRFRAAVPSGASTGVHEALELRDGDKSRYLGKGTVKAVDHVNKDIAAKLIEKKFSVVDQEKIDHFMLELDGTENKSKFGANAILGVSLAVCKAGAAEKGVPLYRHIADLAGHKDVILPCPAFNVINGGSHAGNKLAMQEFMILPIGASNFHEAMRIGAEVYHNLKNVIKAKYGKDATNVGDEGGFAPNILENNEALELLKTAIEKAGYPDKIIIGMDVAASEFYKAGKYDLDFKSPDDPARYITGDQLGDLYKSFIKGYPVQSIEDPFDQDDWAAWTKFTAAVDIQVVGDDLTVTNPKRIQQAVEKKACNCLLLKVNQIGSVTESIKACKLAQSNGWGVMVSHRSGETEDTFIADLVVGLCTGQIKTGAPCRSERLAKYNQLMRIEEELGAKAKFAGKDYRHPKIN

SEQ ID NO: 212 - protein, 433 aa, Cyprinus carpio
MSISKIHAREILDSRGNPTVEVDLYTAKGRFRAAVPSGASTGVHEALELRDGDKTRYLGKGTQKAVDHVNKEIAPKLIEKKFSVVEQEKIDKFMLELDGTENKSKFGANAILGVSLAVCKAGAAEKGVPLYRHIADLAGNKDVILPVPAFNVINGGSHAGNKLAMQEFMILPVGAKNFHEAMRIGAEVYHNLKNVIKAKYGKDATNVGDEGGFAPNILENNEALELLKSAIEKAGYPDKIIIGMDVAASEFFKNGKYDLDFKSPDDPKRHITGDQLGDLYKSFIKNYPVQSIEDPFDQDDWENWSKFTGSVDIQVVGDDLTVTNPKRIQQACEKKACNCLLLKVNQIGSVTESIQACKLAQSNGWGVMVSHRSGETEDTFIADLVVGLCTGQIKTGAPCRSERLAKYNQLMRIEEELGDKAKFAGKDFRHPKL

SEQ ID NO: 213 - protein, 434 aa, Homo sapiens
MSIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 214 - protein, 434 aa, Salmo salar
MSIVSIIAREILDSRGNPTVEVDLCTEKGLFRAAVPSGASTGIYEALELRDGDKSRYKGKGVLKAVGHINDTLAPALIASGISVVEQEQLDNTMIEMDGTENKSQFGANAILGVSLAICKAGAAEKEVPLYRHIADLAGNTELVLPVPAFNVINGGSHAGNKLAMQEFMVLPVGAESFRDALRVGSELYHTLRGVIQEKYGQDATNVGDEGGFAPNILENSEALELIKTAIEKAGFTDKVVIGMDVAASEFYREGKYDLDFKSPPDAERHISGEELSDIYQGFVNDYPVVSIEDPFDQDDWAAWSRLTASVGIQVVGDDLTVTNPKRIEKAAEERACNCLLLKVNQIGSVTEAIQACKLAQANGWGVMVSHRSGETEDTFIADLVVGLCTGQIKTGAPCRSERLAKYNQLMRIEEELGDQARFAGYNFRNPTAL

SEQ ID NO: 215 - protein, 284 aa, Penaeus aztecus
MDAIKKKMQAMKLEKDNAMDRADTLEQQNKEANNRAEKSEEEVHNLQKRMQQLENDLDQVQESLLKANIQLVEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 216 - protein, 284 aa, Penaeus monodon; identical to SEQ ID NO: 215

SEQ ID NO: 217 - protein, 284 aa, Homarus americanus
MDAIKKKMQAMKLEKDNAMDRADTLEQQNKEANIRAEKTEEEIRITHKKMQQVENELDQVQEQLSLANTKLEEKEKALQNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLANKLKAAEARAEFAERSVQKLQKEVDRLEDELVNEKEKYKSITDELDQTFSELSGY

SEQ ID NO: 218 - protein, 285 aa, Homo sapiens
MMEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 219 - protein, 264 aa, Charybdis feriata
MDAIKKKMQAMKLEKDNAMDRADTLEQQNKEANLRAEKTEEEIRATQKKMQQVENELDQAQEQLSAANTKLDEKEKALQNAEGEVAALNRRIQLPEEDLERSEERLNTATTKLAEASQAADESERMRKVLENRSLSDEERMDALENQLKEARFLAEEADRKYDEVARKLAMVEADLERAEERAESGESKIVELEEELRVVGNNLKSLEVSEEKANQREETYKEQIKTLANKLKAAEARAEFAERSVQKLQKEVDRLEDELVNEK

SEQ ID NO: 220 - protein, 324 aa, Penaeus vannamei
KEVFDKLKDKRTSLGATLLDVIQSGVENLDSGVGIYAPDAEAYTLFAPLFDPIIEDYHVGFKQTDKHPNKDFGDVNSFVNVDPEGKFVISTRVRCGRSLQGYPFNPCLTESQYKEMEAKVSSTLSSLEGELKGTYYPLTGMSKEVQQKLIDDHFLFKEGDRFLQAANACRYWPAGRGIYHNDNKTFLVWVNEEDHLRIISMQMGGDLGQVFRRLTSAVNEIEKRIPFSHHDRLGFLTFCPTNLGTTVRASVHIKLPKLAANREKLEEVAGKYNLQVRGTRGEHTEAEGGIYDISNKRRMGLTEFQAVKEMQDGILELIKIEKEM

SEQ ID NO: 221 - protein, 328 aa, Homo sapiens
KDEDDVDQELANIDPTWIESPKTNGHIENGPFSLEQQLDDEDDDEEDCPNPEEYNLDEPNAESDYTYSSSYEQFNGELPNGRHKIPESQFPEFSTSLFSGSLEPVACGSVLSEGSPLTEQEESSPSHDRSRTVSASSTGDLPKAKTRAADLLVNPLDPRNADKIRVKIADLGNACWVHKHFTEDIQTRQYRSIEVLIGAGYSTPADIWSTACMAFELATGDYLFEPHSGEDYSRDEDHIAHIIELLGSIPRHFALSGKYSREFFNRRGELRHITKLKPWSLFDVLVEKYGWPHEDAAQFTDFLIPMLEMVPEKRASAGECLRHPWLNS

SEQ ID NO: 222 - protein, 325 aa, Limulus polyphemus
KDVFDSIKNKKTGMGATLLDVIQSGVENLDSGVGIYAPDAESYRTFGPLFDPIIDDYHGGFKLTDKHPPKEWGDINTLVDLDPGGQFIISTRVRCGRSLQGYPFNPCLTAEQYKEMEEKVSSTLSSMEDELKGTYYPLTGMSKATQQQLIDDHFLFKEGDRFLQTANACRYWPTGRGIFHNDAKTFLVWVNEEDHLRIISMQKGGDLKTVYKRLVTAVDNIESKLPFSHDDRFGFLTFCPTNLGTTMRASVHIQLPKLAKDRKVLEDIASKFNLQVRGTRGEHTESEGGVYDISNKRRLGLTEYQAVREMQDGILEMIKMEKAAA

SEQ ID NO: 223 - protein, 324 aa, Penaeus monodon
KAVFDQLKEKKTSLGATLLDVIQSGVENLDSGVGIYAPDAEAYTLFSPLFDPIIEDYHVGFKQTDKHPNKDFGDVNTFVNVDPEGKYVISTRVRCGRSMEGYPFNPCLTEAQYKEMEAKVSSTLSSLEGELKGTYYPLTGMSKEVQQKLIDDHFLFKEGDRFLQAANACRYWPAGRGIYHNDNKTFLVWVNEEDHLRIISMQMGGDLGQVFRRLTSAVNEIEKRIPFSHHDRLGFLTFCPTNLGTTVRASVHIKLPKLAANREKLEEVAGKYNLQVRGTRGEHTEAEGGIYDISNKRRMGLTEFQAVKEMQDGILELIKMEKEM

SEQ ID NO: 224 - protein, 324 aa, Homarus gammarus
KDIFDSLKAKKTSLGATLLDVIQSGVENLDSGVGIYAPDAEAYSLFAPLFDPIIEDYHKGFKQTDKHPAKDFGDVSKFINVDPEGTFVISTRVRCGRSMEGYPFNPCLTEAQYKEMEEKVSSTLSGLEGELKGSYFPLTGMTKEVQQKLIDDHFLFKEGDRFLQAANACRYWPAGRGIYHNDNKTFLVWCNEEDHLRIISMQMGGDLGQVYRRLVSAVNDIEKRVPFSHHDRLGFLTFCPTNLGTTVRASVHIKLPKLAANREKLEEVAAKFSLQVRGTRGEHTEAEGGIYDISNKRRMGLTEFQAVKEMQDGILELIKIEKEM

SEQ ID NO: 225 - protein, 564 aa, Carya illinoinensis
NDDEENPRDPREQYRQCQEHCRRQGQGQRQQQQCQSRCEERLEEEQRKQEERERRRGRDEDDQNPRDPEQRYEQCQQQCERQRRGQEQQLCRRRCEQQRQQEERERQRGRDRQDPQQQYHRCQRRCQTQEQSPERQRQCQQRCERQYKEQQGREWGPDQASPRRESRGREEEQQRHNPYYFHSQGLRSRHESGEGEVKYLERFTERTELLRGIENYRVVILEANPNTFVLPYHKDAESVIVVTRGRATLTFVSQERRESFNLEYGDVIRVPAGATEYVINQDSNERLEMVKLLQPVNNPGQFREYYAAGAQSTESYLRVFSNDILVAALNTPRDRLERFFDQQEQREGVIIRASQEKLRALSQHAMSAGQRPWGRRSSGGPISLKSQRSSYSNQFGQFFEACPEEHRQLQEMDVLVNYAEIKRGAMMVPHYNSKATVVVYVVEGTGRFEMACPHDVSSQSYEYKGRREQEEEESSTGQFQKVTARLARGDIFVIPAGHPIAITASQNENLRLVGFGINGKNNQRNFLAGQNNIINQLEREAKELSFNMPREEIEEIFERQVESY

SEQ ID NO: 226 - protein, 562 aa, Juglans regia
DDDEENPRDPREQYRQCQEYCRRQGQGQRQQQQCQIRCEERLEEDQRSQEERERRRGRDVDDQNPRDPEQRYEQCQQQCERQRRGQEQTLCRRRCEQRRQQEERERQRGRDRQDPQQQYHRCQRRCQIQEQSPERQRQCQQRCERQYKEQQGRERGPEASPRRESRGREEEQQRHNPYYFHSQSIRSRHESEEGEVKYLERFTERTELLRGIENYRVVILDANPNTSMLPHHKDAESVAVVTRGRATLTLVSQETRESFNLECGDVIRVPAGATVYVINQDSNERLEMVKLLQPVNNPGQFREYYAAGAKSPDQSYLRVFSNDILVAALNTPRDRLERFFDQQEQREGVIIRASQEKLRALSQHAMSAGQRPWGRRSSGGPISLKSESPSYSNQFGQFFEACPEEHRQLQEMDVLVNYAEIKRGAMMVPHYNSKATVVVYVVEGTGRYEMACPHVSSQSYEGQGRREQEEEESTGRFQKVTARLARGDIFVIPAGHPIAITASQNENLRLLGFDINGENNQRDFLAGQNNIINQLEREAKELSFNMPREEIEEIFESQMESY

SEQ ID NO: 227 - protein, 583 aa, Homo sapiens
EEEQQQQRLRREQQLRRKQEEERREQQEERREQQERREQQEERREQQLRREQEERREQQLRREQEEERREQQLRREQEEERREQQLRREQQLRREQQLRREQQLRREQQLRREQQLRREQQLRREQQLRREQQLRREQEEERHEQKHEQERREQRLKREQEERRDWLKREEETERHEQERRKQQLKRDQEEERRERWLKLEEEERREQQERREQQLRREQEERREQRLKRQEEEERLQQRLRSEQQLRREQEERREQLLKREEEKRLEQERREQRLKREQEERRDQLLKREEERRQQRLKREQEERLEQRLKREEVERLEQEERREQRLKREEPEEERRQQLLKSEEQEERRQQQLRREQQERREQRLKREEEEERLEQRLKREHEEERREQELAEEEQEQARERIKSRIPKWQWQLESEADARQSKVYSRPRKQEGQRRRQEQEEKRRRRESELQWQEEERAHRQQQEEEQRRDFTWQWQAEEKSERGRQRLSARPPLREQRERQLRAEERQQREQRFLPEEEEKEQRRRQRREREKELQFLEEEEQLQRRERAQQLQEEEDGLQEDQERRRSQEQRRDQKW

SEQ ID NO: 228 - protein, 410 aa, Anacardium occidentale
EHEEEEEEWGTGGVDEPSTHEPAEKHLSQCMRQCERQEGGQQKQLCRFRCQERYKKERGQHNYKREDDEDEDEDEAEEEDENPYVFEDEDFTTKVKTEQGKVVLLPKFTQKSKLLHALEKYRLAVLVANPQAFVVPSHMDADSIFFVSWGRGTITKILENKRESINVRQGDIVSISSGTPFYIANNDENEKLYLVQFLRPVNLPGHFEVFHGPGGENPESFYRAFSWEILEAALKTSKDTLEKLFEKQDQGTIMKASKEQIRAMSRRGEGPKIWPFTEESTGSFKLFKKDPSQSNKYGQLFEAERIDYPPLEKLDMVVSYANITKGGMSVPFYNSRATKIAIVVSGEGCVEIACPHLSSSKSSHPSYKKLRARIRKDTVFIVPAGHPFATVASGNENLEIVCFEVNAEGN

SEQ ID NO: 229 - protein, 417 aa, Pistacia vera
EEEEEEEWGSGRGRGDEFSTHEPGEKRLSQCMKQCERQDGGQQKQLCRFRCQEKYKKERREHSYSRDEEEEEEGDEEQEEEDENPYVFEDEHFTTRVKTEQGKVVVLPKFTKRSKLLRGLEKYRLAFLVANPQAFVVPNHMDADSIFFVSWGRGTITKIRENKRESMNVKQGDIIRIRAGTPFYIVNTDENEKLYIVKLLQPVNLPGHYEVFHGPGGENPESFYRAFSREVLEAALKTPRDKLEKLFEKQDEGAIVKASKEQIRAMSRRGEGPSIWPFTGKSTGTFNLFKKDPSQSNNYGQLFESEFKDYPPLQELDIMVSYVNITKGGMSGPFYNSRATKIAIVVSGEGRLEIACPHLSSSKNSGQEKSGPSYKKLSSSIRTDSVFVVPAGHPFVTVASGNQNLEILCFEVNAEGN

SEQ ID NO: 230 - protein, 546 aa, Homo sapiens
HTTDSSLEEKQRTLDSGTSEIVKSPRIECSKTRREMQSVVQLIMTRDSDGYENSTDGEMCDKDALEEDSESVSEIGSDEESENEITSVGRASGDDDGSEDDEEEDEDEEEDEDEDSEDDDKSDSGPDLARGKGNIETSSEDEDDTADLFPEESGFEHAWRELDKDAPRADEITRRLAVCNMDWDRLKAKDLLALFNSFKPKGGVIFSVKIYPSEFGKERMKEEQVQGPVELLSIPEDAPEKDWTSREKLRDYQFKRLKYYYAVVDCDSPETASKIYEDCDGLEFESSCSFIDLRFIPDDITFDDEPKDVASEVNLTAYKPKYFTSAAMGTSTVEITWDETDHERITMLNRKFKKEELLDMDFQAYLASSSEDEEEIEEELQGDDGVNVEEDGKTKKSQKDDEEQIAKYRQLLQVIQEKEKKGKENDMEMEIKWVPGLKESAEEMVKNKLEGKDKLTPWEQFLEKKKEKKRLKRKQKALAEEASEEELPSDVDLNDPYFAEEVKQIGINKKSVKSAKDGTSPEEEIEIERQKAEMALLMMDEDEDSK

SEQ ID NO: 231 - protein, 534 aa, Macadamia integrifolia
KEICEEEEEYNRQRDPQQQYEQCQKRCQRRETEPRHMQICQQRCERRYEKEKRKQQKRYEEQQREDEEKYEERMKEGDNKRDPQQREYEDCRRHCEQQEPRLQYQCQRRCQEQQRQHGRGGDLMNPQRGGSGRYEEGEEKQSDNPYYFDERSLSTRFRTEEGHISVLENFYGRSKLLRALKNYRLVLLEANPNAFVLPTHLDADAILLVIGGRGALKMIHRDNRESYNLECGDVIRIPAGTTFYLINRDNNERLHIAKFLQTISTPGQYKEFFPAGGQNPEPYLSTFSKEILEAALNTQTERLRGVLGQQREGVIIRASQEQIRELTRDDSESRRWHIRRGGESSRGPYNLFNKRPLYSNKYGQAYEVKPEDYRQLQDMDVSVFIANITQGSMMGPFFNTRSTKVVVVASGEADVEMACPHLSGRHGGRGGGKRHEEEEEVHYEQVRARLSKREAIVVLAGHPVVFVSSGNENLLLFAFGINAQNNHENFLAGRERNVLQQIEPQAMELAFAASRKEVEELFNSQDESIFFPGP

SEQ ID NO: 232 - protein, 627 aa, Homo sapiens
ANKERSEALRRQQLEQQQRENEEHKRQLLAERQKRIEEQKEQRRRLEEQQRREKELRKQQEREQRRHYEEQMRREEERRRAEHEQEYIRRQLEEEQRQLEILQQQLLHEQALLLEYKRKQLEEQRQAERLQRQLKQERDYLVSLQHQRQEQRPVEKKPLYHYKEGMSPSEKPAWAKEVEERSRLNRQSSPAMPHKVANRISDPNLPPRSESFSISGVQPARTPPMLRPVDPQIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPASYKKAIDEDLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSP

SEQ ID NO: 233 - protein, 497 aa, Prunus dulcis
QHEQEQQCKQRCQPQHGHRQQQQCQQECYEQIRQQREREEMQQMCQQSCEMQGSGGQQQQRQQCQRQCEQQVEQLKQCQLRCQMQGQGQQQEQCQRTCRQELEDQQRRQQQEQGPGGGDNKLEDETLNGEPRRRFEQCMQGCERQQQGQGEQEQCPRQCREQFDKEKRQYQQCKQSCEKSSQHDDEKRQCKQQCKQQISLQQGGGNQEEDDDALNGSGYGGAKERFEQCKLSCQSQQGQGQQEQCPKQCKQQFEHEKHQYKQCKRGCENQARDDVQKEQCKQQCTQQMSQQYGQQQEGSTGGGGLNQQQEEEGQMGSQGQSQSQSQSNNPFYFPSHRFQPRFQSNEGGLYVLERFTQRQSEVLRGIKNYRLAIFEARPNTFVLPHHCDAEAIYVVLSGQCTCTLLMQDRKESFNMEHGDVIRVPAGATTYLVNNNSDKTLRIAKLLQPVNNPGRFEEFFPAGSRNSESYFSVFSNDILESAFNTPREQLEHGFKQGQ

SEQ ID NO: 234 - protein, 622 aa, Homo sapiens
LREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKS

SEQ ID NO: 235 - protein, 490 aa, Cocos nucifera
MVSAMGDRTASAILALLLLSSWSLMVVMAYQGRGMEGREKRVEEKAPRSPEDRGLFILRRSKEVLKTDAGEVRLMMGYRYRGNHSPMHIGFITMEPNTLYIPQYIDASLILFVRRGEAKVGYIYKNKLVEKRLKSGYVDAIPAGSSFYIVNSDKSERLHIICSIDTLESMEYYGAYPQSFYVGGGVLPRSVLSGFDASTLSAAFNVSSDQLEMILNSQRGGPIIRLNGEAAEQRDYLSSIMQLKERMVQEKEMDSDDDDEAEQEEDDVWTWRCLLKSLLGKGDCRVQRKHKLAHSPGSYNLYDTEPSYQNDYGYSIAIDGHDYSALKHSNLGVYLVNLKAGAMLSPHVNPTATEYGIILRGSGTIQVVFPNGTAAMNAKVSEGDVFWIPRYFPFCQIASDGAPLEFFGFTTSARKNRPQFLVGKSSLLHSMRGPELAAAFGVSEEQLERIVKAQREAVILPTSPHREMEEINGKKWKGEEVLVMKRGLFA

SEQ ID NO: 236 - protein, 538 aa, Homo sapiens
MAGPPALPPPETAAAATTAAAASSSAASPHYQEWILDTIDSLRSRKARPDLERICRMVRRRHGPEPERTRAELEKLIQQRAVLRVSYKGSISYRNAARVQPPRRGATPPAPPRAPRGAPAAAAAAAPPPTPAPPPPPAPVAAAAPARAPRAAAAAATAPPSPGPAQPGPRAQRAAPLAAPPPAPAAPPAVAPPAGPRRAPPPAVAAREPPLPPPPQPPAPPQQQQPPPPQPQPPPEGGAVRAGGAARPVSLREVVRYLGGSGGAGGRLTRGRVQGLLEEEAAARGRLERTRLGALALPRGDRPGRAPPAASARPSRSKRGGEERVLEKEEEEDDDEDEDEEDDVSEGSEVPESDRPAGAQHHQLNGERGPQSAKERVKEWTPCGPHQGQDEGRGPAPGSGTRQVFSMAAMNKEGGTASVATGPDSPSPVPLPPGKPALPGADGTPFGCPPGRKEKPSDPVEWTVMDVVEYFTEAGFPEQATAFQEQEIDGKSLLLMQRTDVLTGLSIRLGPALKIYEHHIKVLQQGHFEDDDPDGFLG

SEQ ID NO: 237 - protein, 500 aa, Carya illinoinensis
MAKPILLSIYLCLIIVALFNGCLAQSGGRQQHKFGQCQLNRLDALEPTNRIEAEAGVIESWDPNHQQLQCAGVAVVRRTIEPNGLLLPHYSNAPQLVYIARGRGITGVLFPGCPETFEESQRQSQQGQRREFQQDRHQKIRHFREGDIIAFPAGVAHWCYNDGSSPVVAIFLLDTHNNANQLDQNPRNFYLAGNPDDEFRPQGQQEYEQHRRQQQHQQRRGEHGEQQRDLGNNVFSGFDAEFLADAFNVDTETARRLQSENDHRGSIVRVEGRQLQVIRPRWSREEQEHEERKERERERESESERRQSRRGGRDDNGLEETICTLSLRENIGDPSRADIYTEEAGRISTVNSHNLPILRWLQLSAERGALYSDALYVPHWNLNAHSVVYALRGRAEVQVVDNFGQTVFDDELREGQLLTIPQNFAVVKRARDEGFEWVSFKTNENAMVSPLAGRTSAIRALPEEVLVNAFQIPREDARRLKFNRQESTLVRSRSRSSRSE

SEQ ID NO: 238 - protein, 505 aa, Juglans nigra
MAKPILLSISLCLVALVNGCLAQSGGRQQPRFGECKLKRLVALEPSNRIEAEAGVIESWDPNNQQFQCAGVAVVRRTIEPNGLLLPQYSNAPQLLYIVKGRGITGVLFPGCPETFEESQQGQSRIRPSLRSASFQRDRHQKIRHFREGDVIAFPAGVAHWCYNDGDTPVVAVALMDTTNNANQLDQNPRNFYLAGNPDDEFRPQGQQEYEQHRRQQQHQQRHGEPGQQQRGSGNNVFSGFDADFLADAFNVDTETARRLQSENDHRRSIVRVEGRQLQVIRPRWSREEQEREERKERERERESESERRQSRRGGRDDNGLEETICTLRLRENIGDPSRADIYTEEAGRISTANSHTLPVLRWLQLSAERGALYSDALYVPHWNLNAHSVVYALRGRAEVQVVDNFGQTVFDDELREGQLLTIPQNFAVVKRARNEGFEWVSFKTNENAMVSPLAGRTSAIRALPEEVLANALQIPREDARRLKFNRQESTLVRSRPSSSRSSRSE

SEQ ID NO: 239 - protein, 529 aa, Castanea sativa
MAKLILLYTSLCLLVLFNECLATKTSGQQQQFNQCQLDRLDALEPNNRIKAEAGVIESWDPNDRQFQCVGVAVVRRTIEPNGLLLPQYDSAPQLIYIQSGYGILGAVLPGCPNTYQESQQQQHQQGQQPDQHQKIRTFRLGDIIALPAGVAYWLYNDGDSEVVALSLLDTNNQANQLDKNPRHFYLAGNPEDEFQLQGRSPRGQRRQQQQGQGRRERGQQQQGQGNNLFSGFRTEDLADAFNVDEETIRNLQGFQEDRKNIVKVKGRLQVARPPRSREERERLERQEREQEREDEREQQRESRRGGREEREREERQQEREEEREQERERRESQRGGRGRDNGIEETLCTLRLRENIHDPSRADVYNPQAGRISTLNSHNLPVLRWLQLSAEFGRLQRDAIYVPHWNRNAHSVIYVVKGRAQVQVVDDFGQAVFDDELQQGQILTVPQNFAVVKRASSSEGFEWVAFKTNDNAQISPLAGQNSVLRAIPADVLANAFQLSQEDVSELKSNLDQQEITIVRPYRSSSQRKA

SEQ ID NO: 240 - protein, 457 aa, Anacardium occidentale
LSVCFLILFHGCLASRQEWQQQDECQIDRLDALEPDNRVEYEAGTVEAWDPNHEQFRCAGVALVRHTIQPNGLLLPQYSNAPQLIYVVQGEGMTGISYPGCPETYQAPQQGRQQGQSGRFQDRHQKIRRFRRGDIIAIPAGVAHWCYNEGNSPVVTVTLLDVSNSQNQLDRTPRKFHLAGNPKDVFQQQQQHQSRGRNLFSGFDTELLAEAFQVDERLIKQLKSEDNRGGIVKVKDDELRVIRPSRSQSERGSESEEESEDEKRRWGQRDNGIEETICTMRLKENINDPARADIYTPEVGRLTTLNSLNLPILKWLQLSVEKGVLYKNALVLPHWNLNSHSIIYGCKGKGQVQVVDNFGNRVFDGEVREGQMLVVPQNFAVVKRAREERFEWISFKTNDRAMTSPLAGRTSVLGGMPEEVLANAFQISREDARKIKFNNQQTTLTSGESSHHMRDDA

SEQ ID NO: 241 - protein, 472 aa, Pistacia vera
MGYSSLLSFSLGFLLLFHCSFAQIEQVVNSQQRQQQQRFQTQCQIQNLNALEPKRRIESEAGVTEFWDQNEEQLQCANVAVFRHTIQSRGLLVPSYNNAPELVYVVQGSGIHGAVFPGCPETFQEESQSQSRSQHSRSERSQQSGEQHQKVRHIREGDIIALPAGVAHWIYNNGQSKLVLVALADVGNSENQLDQYLRKFVLGGSPQQEIQGGGQSWSQSRSSRKGQQSNNILSAFDEEILAQSLNIDTQLVKKLQREEKQRGIIVRVKEDLQVLSPQRQEKEYSDNGLEETFCTMTLKLNINDPSRADVYNPRGGRVTSINALNLPILRFLQLSVEKGVLYQNAIMAPHWNMNAHSIVYITRGNGRMQIVSENGESVFDEEIREGQLVVVPQNFAVVKRASSDGFEWVSFKTNGLAKISQLAGRISVMRGLPLDVIQNSFDISREDAWNLKESRSEMTIFAPGSRSQRQRN

SEQ ID NO: 242 - protein, 449 aa, Prunus dulcis
PQLIYIVQGRGVLGAVFSGCPETFEESQQSSQQGRQQEQEQERQQQQQGEQGRQQGQQEQQQERQGRQQGRQQQEEGRQQEQQQGQQGRPQQQQQFRQFDRHQKTRRIREGDVVAIPAGVAYWSYNDGDQELVAVNLFHVSSDHNQLDQNPRKFYLAGNPENEFNQQGQSQPRQQGEQGRPGQHQQPFGRPRQQEQQGSGNNVFSGFNTQLLAQALNVNEETARNLQGQNDNRNQIIRVRGNLDFVQPPRGRQEREHEERQQEQLQQERQQQGGQLMANGLEETFCSLRLKENIGNPERADIFSPRAGRISTLNSHNLPILRFLRLSAERGFFYRNGIYSPHWNVNAHSVVYVIRGNARVQVVNENGDAILDQEVQQGQLFIVPQNHGVIQQAGNQGFEYFAFKTEENAFINTLAGRTSFLRALPDEVLANAYQISREQARQLKYNRQE

SEQ ID NO: 243 - protein, 140 aa, Homo sapiens
MAGWQSYVDNLMCDGCCQEAAIVGYCDAKYVWAATAGGVFQSITPIEIDMIVGKDREGFFTNGLTLGAKKCSVIRDSLYVDGDCTMDIRTKSQGGEPTYNVAVGRAGRVLVFVMGKEGVHGGGLNKKAYSMAKYLRDSGF

SEQ ID NO: 244 - protein, 133 aa, Corylus avellana
MSWQTYVDEHLMCDIDGQGQQLAASAIVGHDGSVWAQSSSFPQLKPEEITGIMKDFDEPGHLAPTGLHLGGTKYMVIQGEAGAVIRGKKGSGGITIKKTGQALVFGIYEEPVTPGQCNMVVERLGDYLIDQGL

SEQ ID NO: 245 - protein, 147 aa, Corylus avellana
MARLATLAALFAALLLVAHAAAFRTTITTVDVDEDIVNQQGRRGESCREQAQRQQNLNQCQRYMRQQSQYGSYDGSNQQQQQELEQCCQQLRQMDERCRCEGLRQAVMQQQGEMRGEEMREVMETARDLPNQCRLSPQRCEIRSARF

SEQ ID NO: 246 - protein, 450 aa, Homo sapiens
MSDFSEELKGPVTDDEEVETSVLSGAGMHFPWLQTYVETVAIGGKRRKDFAQTTSACLSFIQEALLKHQWQQAAEYMYSYFQTLEDSDSYKRQAAPEIIWKLGSEILFYHPKSNMESFNTFANRMKNIGVMNYLKISLQHALYLLHHGMLKDAKRNLSEAETWRHGENTSSREILINLIQAYKGLLQYYTWSEKKMELSKLDKDDYAYNAVAQDVFNHSWKTSANISALIKIPGVWDPFVKSYVEMLEFYGDRDGAQEVLTNYAYDEKFPSNPNAHIYLYNFLKRQKAPRSKLISVLKILYQIVPSHKLMLEFHTLLRKSEKEEHRKLGLEVLFGVLDFAGCTKNITAWKYLAKYLKNILMGNHLAWVQEEWNSRKNWWPGFHFSYFWAKSDWKEDTALACEKAFVAGLLLGKGCRYFRYILKQDHQILGKKIKRMKRSVKKYSIVNPRL

SEQ ID NO: 247 - protein, 138 aa, Anacardium occidentale
MAKFLLLLSAFAVLLLVANASIYRAIVEVEEDSGREQSCQRQFEEQQRFRNCQRYVKQEVQRGGRYNQRQESLRECCQELQEVDRRCRCQNLEQMVRQLQQQEQIKGEEVRELYETASELPRICSISPSQGCQFQSSY

SEQ ID NO: 248 - protein, 239 aa, Homo sapiens
GGGAAVPGAPAAVVVAPPGYVVVQELMVLPAVAAPAVVAIPGPAGSAALTPARQRRRRRARDRPTICGECGKGFSRSTDLVRHQATHTGERPHRCGECGKGFSQHSNLVTHQRIHTGEKPYACSYCAKRFSESSALVQHQRTHTGERPYACGDCGKRFSVSSNLLRHRRTHSGERPYVCEDCGERFRHKVQIRRHERQLHGAGRSRGLGLLRASRPAALGGPARAEQAATATAPADKAL

SEQ ID NO: 249 - protein, 115 aa, Corylus avellana
MGSLKLVCAVLLCMMVAAPVARASLTCPQIKGNLTPCVLYLKNGGVLPPSCCKGVRAVNDASRTTSDRQSACNCLKDTAKGIAGLNPNLAAGLPGKCGVNIPYKISPSTNCNNVK

SEQ ID NO: 250 - protein, 292 aa, Homo sapiens
MAASTMSIRSSAYSDSWQVDDCPESCCEPPCCATSCCAPAPCLTLVCTPVSRVSSPCCQVTCEPSPCQSGCTSSCTPSCCQQSSCQPAYCTSSPCQQACCVPVCCKPVCCVPVCCGASSCCQQSSYQPACCASSSCQPACCVPVCCKPVCCAPTCSEDSYSCCQQSSCQPACCTSSPCQQSYCVPVCCKPVCCKPICCVPVCSGASSLCCQQSGCQPACCTTSCCRPSSSVSLLCRPVCRPACCVPVSSCCAPTSSRQPSYCRQASCVSLLCRPVCSRPACYSFSSGQKSSC

SEQ ID NO: 251 - protein, 517 aa, Pisum sativum
MAKLLALSLSFCFLLLGGCFALREQPQQNECQLERLDALEPDNRIESEGGLIETWNPNNKQFRCAGVALSRATLQRNALRRPYYSNAPQEIFIQQGNGYFGMVFPGCPETFEEPQESEQGEGRRYRDRHQKVNRFREGDIIAVPTGIVFWMYNDQDTPVIAVSLTDIRSSNNQLDQMPRRFYLAGNHEQEFLQYQHQQGGKQEQENEGNNIFSGFKRDYLEDAFNVNRHIVDRLQGRNEDEEKGAIVKVKGGLSIISPPEKQARHQRGSRQEEDEDEEKQPRHQRGSRQEEEEDEDEERQPRHQRRRGEEEEEDKKERGGSQKGKSRRQGDNGLEETVCTAKLRLNIGPSSSPDIYNPEAGRIKTVTSLDLPVLRWLKLSAEHGSLHKNAMFVPHYNLNANSIIYALKGRARLQVVNCNGNTVFDGELEAGRALTVPQNYAVAAKSLSDRFSYVAFKTNDRAGIARLAGTSSVINNLPLDVVAATFNLQRNEARQLKSNNPFKFLVPARESENRASA

SEQ ID NO: 252 - protein, 496 aa, Cicer arietinum
MAKLLALSLSFCFLLFGTCFALRDQPQQNECQLEHLNALKPDNRIKSEGGLIETWNPSNKQFACAGVALSRATLQPNSLLQTFLHQRSPEIFIQQGNGYFGMVFPGCVETFEEPRESEQGEGSKFSDSHQKVNRFREGDIIAVPTGVVFWMFNDQDTPVIAVSLIDTSSFQNQLDQMPRRFYLAGNHEQEFLRYQQEGSEEEENEGGNIFSGFKRDFLEDALNVNRRIVNKLQGRNEDEEKGAIVKVKGGLSITTPPEKEPRQKRGSRQEEDEDEDEKRQPHRHSRQDEDEDEKRQPHHHSRGGSKSQRDNGFEETICTARLHQNIGSSSSPDIYNPQAGRIKTVTSFDLQALRFLKLSAEFGSLHKNAMFVPHYNLNANSILYALKGRARLLYALNCKGNSVFDGELEAGRALIVPQNFAIAAKSLSDRFSYVAFKTNDRALINVCQKKLLQLLSIWKEMRPGSSSSTAPFHFLFHPAVTQTTKQQLDLVPNQYE

SEQ ID NO: 253 - protein, 352 aa, Glycine max
VVAISPLDTSNFNNQLDQNPRVFYLAGNPDIEHPETMQQQQQQKSHGGRKQGQHRQQEEEGGSVLSGFSKHFLAQSFNTNEDTAEKLRSPDDERKQIVTVEGGLSVISPKWQEQEDEDEDEDEEYGRTPSYPPRRPSHGKHEDDEDEDEEEDQPRPDHPPQRPSRPEQQEPRGRGCQTRNGVEENICTMKLHENIARPSRADFYNPKAGRISTLNSLTLPALRQFGLSAQYVVLYRNGIYSPDWNLNANSVTMTRGKGRVRVVNCQGNAVFDGELRRGQLLVVPQNPAVAEQGGEQGLEYVVFKTHHNAVSSYIKDVFRVIPSEVLSNSYNLGQSQVRQLKYQGNSGPLVNP

SEQ ID NO: 254 - protein, 9 aa, Homo sapiens
YCQQYDNLP

SEQ ID NO: 255 - protein, 313 aa, Arachis hypogaea
VVAISVLHTNSLHNQLDQFPRRFNLAGKQEQEFLRYQQRSGRQSPKGEEQEQEQENEGGNVFSGFSTEFLSHGFQVNEDIVRNLRGENEREEQGAIVTVKGGLSILVPPEWRQSYQQPGRGDKDFNNGIEETICTATVKMNIGKSTSADIYNPQAGSVRTVNELDLPILNRLGLSAEYGSIHRDAMFVPHYNMNANSMIYALHGGAHVQVVDCNGNRVFDEELQEGQSLVVPQNFAVAAKSQSEHFLYVAFKTNSRASISNLAGKNSYMWNLPEDVVANSYGLQYEQARQLKNNNPFTFLVPPQDSQMIRTVA

SEQ ID NO: 256 - protein, 286 aa, Homo sapiens
PQCPTLESQEGENSEEKGDSSKEDPKETVALAFVRENPGAQNGLQNAQQQGKKKRKKKRLVINLSSCRYESVRRAAQQYGFREGGEDDDWTLYWTDYSVSLERVMEMKSYQKINHFPGMSEICRKDLLARNMSRMLKMFPKDFRFFPRTWCLPADWGDLQTYSRSRKNKTYICKPDSGCQGKGIFITRTVKEIKPGEDMICQLYISKPFIIDGFKFDLRIYVLVTSCDPLRIFVYNEGLARFATTSYSRPCTDNLDDICMHLTNYSINKHSSNFSRDAHSGSKRKL

SEQ ID NO: 257 - protein, 566 aa, Arachis hypogaea
RCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 258 - protein, 172 aa, Arachis hypogaea
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 259 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 260 - protein, 119 aa, Arachis hypogaea
RQQPEENACQFQRLNAQRPDNRIESEGGYIETWNPNNQEFECAGVALSRLVLRRNALRRPFYSNAPQEIFIQQGRGYFGLIFPGCPRHYEEPHTQGRRSQSQRPPRRLQGEDQSQQQRD

SEQ ID NO: 261 - protein, 361 aa, Homo sapiens
MWVLTPAAFAGKLLSVFRQPLSSLWRSLVPLFCWLRATFWLLATKRRKQQLVLRGPDETKEEEEDPPLPTTPTSVNYHFTRQCNYKCGFCFHTAKTSFVLPLEEAKRGLLLLKEAGMEKINFSGGEPFLQDRGEYLGKLVRFCKVELRLPSVSIVSNGSLIRERWFQNYGEYLDILAISCDSFDEEVNVLIGRGQGKKNHVENLQKLRRWCRDYRVAFKINSVINRFNVEEDMTEQIKALNPVRWKVFQCLLIEGENCGEDALREAERFVIGDEEFERFLERHKEVSCLVPESNQKMKDSYLILDEYMRFLNCRKGRKDPSKSILDVGVEEAIKFSGFDEKMFLKRGGKYIWSKADLKLDW

SEQ ID NO: 262 - protein, 141 aa, Glycine max
MASTHKVFAVTILLLAVNMASPSSSSSSSNTSLVARLKLDGESSNCWDSLFELQACTGEVITFFLNGETYLGPSCCQAIRIVGHDCWPDMIASLGFTTEEGDVLQGYCDSEKEQLHSPPSPTSPTTVVHDFDDATTKVLLP

SEQ ID NO: 263 - protein, 158 aa, Glycine max
MTKFTILLISLLFCIAHTCSASKWQHQQDSCRKQLQGVNLTPCEKHIMEKIQGRGDDDDDDDDDNHILRTMRGRINYIRRNEGKDEDEEEEGHMQKCCTEMSELRSPKCQCKALQKIMENQSEELEEKQKKKMEKELINLATMCRFGPMIQCDLSSDD

SEQ ID NO: 264 - protein, 848 aa, Triticum aestivum
MAKRLVLFVAVVVALVALTVAEGEASEQLQCERELQELQERELKACQQVMDQQLRDISPECHPVVVSPVAGQYEQQIVVPPKGGSFYPGETTPPQQLQQRIFWGIPALLKRYYPSVTCPQQVSYYPGQASPQRPGQGQQPGQGQQGYYPTSPQQPGQWQQPEQGQPRYYPTSPQQSGQLQQPAQGQQPGQGQQGQQPGQGQPGYYPTSSQLQPGQLQQPAQGQQGQQPGQAQQGQQPGQGQQPGQGQQGQQPGQGQQPGQGQQGQQLGQGQQGYYPTSLQQSGQGQPGYYPTSLQQLGQGQSGYYPTSPQQPGQGQQPGQLQQPAQGQQPGQGQQGQQPGQGQQGQQPGQGQQPGQGQPGYYPTSPQQSGQGQPGYYPTSSQQPTQSQQPGQGQQGQQVGQGQQAQQPGQGQQPGQGQPGYYPTSPQQSGQGQPGYYLTSPQQSGQGQQPGQLQQSAQGQKGQQPGQGQQPGQGQQGQQPGQGQQGQQPGQGQPGYYPTSPQQSGQGQQPGQWQQPGQGQPGYYPTSPLQPGQGQPGYDPTSPQQPGQGQQPGQLQQPAQGQQGQQLAQGQQGQQPAQVQQGQRPAQGQQGQQPGQGQQGQQLGQGQQGQQPGQGQQGQQPAQGQQGQQPGQGQQGQQPGQGQQGQQPGQGQQPGQGQPWYYPTSPQESGQGQQPGQWQQPGQGQPGYYLTSPLQLGQGQQGYYPTSLQQPGQGQQPGQWQQSGQGQHWYYPTSPQLSGQGQRPGQWLQPGQGQQGYYPTSPQQPGQGQQLGQWLQPGQGQQGYYPTSLQQTGQGQQSGQGQQGYYSSYHVSVEHQAASLKVAKAQQLAAQLPAMCRLEGGDALSASQ

SEQ ID NO: 265 - protein, 737 aa, Secale cereale
MAKRLVLFATVVIALVALTAAEGEASGQLQCEHELQESSLEACRQVVDQQLVGRLPWSTGLHMRCCQQLRDVSAKCRHVAVSQVARQYEQTTVPPKGGSFYPSETTPLQQLQQGIFWGTSSQTVQGYYPSVTSPQQGSYYPGRASPQQPGQGQQPGKWQEPGQGQQGYYPTSLQQPGQGQQEHYPASQQQPGQGQQGHYPASLQQPGQGQQTGQPGQIQQPGQGQQIGQGQQPEQEQQPGQGQQGYYPTPPQQPGQGHQPGQGQQEYYPTSLQQPGQGQPGQMQQPGQGQQTGQGQQPEQEQQPGQGQQGYYPASLQEPGQGQQAHYPASQQQPGQGQQGHYPASPQQPGQGRQGHYPASLQQPGQGQQTEQPGQGQQPEQGQQPEQEQQPGQGQQGYYPTSPQQPGQGQQPGQGQQGYYSTSLQQPGQGQQGHYPTSLQQPRQRQQPGQGQQIGQGQQPEQQQQPGQGQQGYYPTYPQQPGQGQQPGQGQQGYYSTSLQQPGQGQQGHYPTSLQQPGQGQPGQRQQPGQGQQIGQGQQPEPEQQPGQGQQGHYPASLQQPGQGQQTEQPGQGQQQGQRQHPEQEQQPGQGQQGYYPTSQQQPGQGQQPGQWQQPGQGKQGHCPTSRQQPGQAQQPGQGRQIGQAQKPGQGQQGHYPTSLQQPGQLQQSGQGQQPGQGHQPGQGQQSGQDQQGHDSPCHVSAEQQATSPKVAKAQQPVAQLPAMCRMEAGDALSATQ

SEQ ID NO: 266 - protein, 220 aa, Avena sativa
MKTFLIFALLAMAATMATAQFDPSEQYQPYPEQQQPILQQQQMLLQQQQQMLLQQQPLLQVLQQQLNPCRQFLVQQCSPVAVVPFLRSQILQQSSCQVMRQQCCRQLEQIPEQLRCPAIHSVVQAIIMQQQQFFQPQMQQQFFQPQMQQVTQGIFQPQMQQVTQGIFQPQLQQVTQGIFQPQMQGQIEGMRAFALQALPAMCDVYVPPHCPVATAPLGGF

SEQ ID NO: 267 - protein, 196 aa, Zea mays
MRVLLVALALLALAASATSTHTSGGCGCQPPPPVHLPPPVHLPPPVHLPPPVHLPPPVHLPPPVHLPPPVHVPPPVHLPPPPCHYPTQPPRPQPHPQPHPCPCQQPHPSPCQLQGTCGVGSTPILGQCVEFLRHQCSPTATPYCSPQCQSLRQQCCQQLRQVEPQHRYQAIFGLVLQSILQQQPQSGQVAGLLAAQ

SEQ ID NO: 268 - protein, 160 aa, Homo sapiens
PPPPPSSYMTGMSTTSSYMSGEGYQSLQSMMKTEGPSYGALPPAYGPPAHLPYHPHVYPPNPPPPPVPPPPASFPPPAIPPPTPGYPPPPPTYNPNFPPPPPRLPPTHAVPPHPPPGLGLPPASYPPPAVPPGGQPPVPPPIPPPGMPPVGGLGRAAWMR

SEQ ID NO: 269 - protein, 214 aa, Secale cereale
MKTLLILTILAMATTIATANMQVDPSGQVQWPQQQPFPQPQQPFPQQPQLPFPQQPQQPLPQPQQPQQPFPQPQQPQQPFPQSQQQFPQPQQPQQSIPQQQQPLIESSLQQQMNPCKNFLLQQCIPVSLVSSLVSLILPRSDCQVMQQQCCQQLAQIPQHLHRAAIHSVVHSIIMQQQQQQQQGLQILRPLFQIVQGQSIIQQQLAQLEAIRSL

SEQ ID NO: 270 - protein, 243 aa, Hordeum brachyantherum
MKILLILTILAMAITITTANVQVDPSGQVQWPQQQPFPQQPQQTFPQPQQTFPHQPQQQFPQPQQPQQPFPQPQQPQQPFPQPQQPQQPFPQPQQPQQPFPQSQQPQQPFPQPQQQFPQPQQPQQSLLQQQQPMIQSFLQQQMNPCKNFLLQQCNPVSLVSSIVSIILPRSDCQVMQQQCCQQLAQIPQQLQCAAIHNVVHSIIMQQEQQQGVQILRPLFQIVQGLGIIQPQKLAQLEAIRSL

SEQ ID NO: 271 - protein, 301 aa, Triticum aestivum
MKTLLIVTILAMATTIATANMQVDPGYQVHWPQQQPFLQPQQPFCQQPQQTIPQPHQTFHHQPQQTFPQPQQTYPHQPQQQFPQTQQPQQPFPQPQQTFPQQPQLPFPQQPQQPFPQPQQPQQQFPQSQQPQQPFPQPQQQFPQPQQPQQSIPQQQQPLIQSSLQQQMNPCKNFLLQQCNPVSLVSSLVSLILPRSDCQVMQQQCCQQLAQIPHHLQCAAIHSVAHSVIMQQQQQQGIQILRPLFQIVQGQSIIQQQPAQLEVIRSLVLKTLPTMCNVYVRPDCSNIRTPFASTIAGIGGQ

SEQ ID NO: 272 - protein, 282 aa, Triticum aestivum
MKTFLILALLAIVATTATSAVRVPVPQLQPQNPSQQQPQEQVPLMQQQQQFPGQQEQFPPQQPYPHQQPFPSQQPYPQPQPFPPQLPYPQTQPFPPQQPYPQPQPQYPQPQQPISQQQAQQQQQQQQTLQQILQQQLIPCRDVVLQQHNIAHASSQVLQQSSYQQLQQLCCQQLFQIPEQSRCQAIHNVVHAIILHHHQQQQQQPSSQVSYQQPQEQYPSGQVSFQSSQQNPQAQGSVQPQQLPQFQEIRNLALQTLPAMCNVYIPPYCSTTIAPFGIFGTN

SEQ ID NO: 273 - protein, 438 aa, Homo sapiens
GNILPPEVRGNLMAAGQNLQSSERSEMIATWSPAVRTLRNITNNADIQQMNRPSNVAHILQTLSAPTKNLEQQVNHSQQGHTNANAVLFSQVKVTPETHMLQQQQQAQQQQQQHPVLHLQPQQIMQLQQQQQQQISQQPYPQQPPHPFSQQQQQQQQAHPHQFSQQQLQFPQQQLHPPQQLHRPQQQLQPFQQQHALQQQFHQLQQHQLQQQQLAQLQQQHSLLQQQQQQQIQQQQLQRMHQQQQQQQMQSQTAPHLSQTSQALQHQVPPQQPPQQQQQQQPPPSPQQHQLFGHDPAVEIPEEGFLLGCVFAIADYPEQMSDKQLLATWKRIIQAHGGTVDPTFTSRCTHLLCESQVSSAYAQAIRERKRCVTAHWLNTVLKKKKMVPPHRALHFPVAFPPGGKPCSQHIISVTGFVDSDRDDLKLMAYLAGAKYTGY

SEQ ID NO: 274 - protein, 519 aa, Homo sapiens
MFLLPLPAAGRVVVRRLAVRRFGSRSLSTADMTKGLVLGIYSKEKEDDVPQFTSAGENFDKLLAGKLRETLNISGPPLKAGKTRTFYGLHQDFPSVVLVGLGKKAAGIDEQENWHEGKENIRAAVAAGCRQIQDLELSSVEVDPCGDAQAAAEGAVLGLYEYDDLKQKKKMAVSAKLYGSGDQEAWQKGVLFASGQNLARQLMETPANEMTPTRFAEIIEKNLKSASSKTEVHIRPKSWIEEQAMGSFLSVAKGSDEPPVFLEIHYKGSPNANEPPLVFVGKGITFDSGGISIKASANMDLMRADMGGAATICSAIVSAAKLNLPINIIGLAPLCENMPSGKANKPGDVVRAKNGKTIQVDNTDAEGRLILADALCYAHTFNPKVILNAATLTGAMDVALGSGATGVFTNSSWLWNKLFEASIETGDRVWRMPLFEHYTRQVVDCQLADVNNIGKYRSAGACTAAAFLKEFVTHPKWAHLDIAGVMTNKDEVPYLRKGMTGRPTRTLIEFLLRFSQDNA

SEQ ID NO: 275 - protein, 598 aa, Oryza sativa
MATAASTSAAAVAAASRLLVRRAPPRLLRRLPRAALAASRPSPPSSSSYGAAAVALGRQPLGHRARMGHTAAAAAAAGPALGLTKPNAVEPPQVSFAAKDVEFSEWKGDILAIAVTENDLVKGSDSKFENAVLKKLDGQLGGLLSEASAEEDFTGKAGQSVVLRLPGQGFKRVGLIGLGQNAPSTTTACKGIGESVASVAKSAQASSAAIVFASVGGIQEDFKLTAAAAIASGTVLGLHEDSRYKSESKKVHLKQVDLIGFGSGPEVDQKLKYANDLSSGVIFGKELVNSPANVLTPAVLAEEASNIASTYSDVFTATILDVEKCKELKMGSYLGVAAASANPPHFIHLCYKPPGGNAKRKLAIVGKGLTFDSGGYNIKTGPGCSIELMKFDMGGSAAVFGAAKALGQIKPPGVEVHFIVAACENMISGTGMRPGDIVTASNGKTIEVNNTDAEGRLTLADALVYACNQGVDKIIDLATLTGACVVALGPSIAGIFTPSDELAKEVAAASEISGEKFWRMPLEESYWESMKSGVADMVNTGGRQGGSITAALFLKQFVDEKVQWMHIDMAGPVWNDKKRAATGFGVSTLVEWVLKNSS

SEQ ID NO: 276 - protein, 131 aa, Zea mays
MSWQTYVDEHLMCEIEGHHLSSAAIVGHDGAVWAQSTAFPQFKPEEMTNIIKDFDEPGFLAPIGLFLGPTKYMVIQGEPGAVIRGKKGSGGITVKKTGQALVIGIYDEPMTPGQCNMVVERLGDYLVEQGL

SEQ ID NO: 277 - protein, 9 aa, Unknown
  Present in Penaeus vannamei, Limulus polyphemus, Penaeus monodon and Homarus gammarus
GYPFNPCLT

SEQ ID NO: 278 - protein, 13 aa, Unknown
  Present in Penaeus vannamei, Limulus polyphemus, Penaeus monodon and Homarus gammarus
GFLTFCPTNLGTT

SEQ ID NO: 279 - protein, 11 aa, Unknown
  Present in Penaeus vannamei, Limulus polyphemus, Penaeus monodon and Homarus gammarus
QVRGTRGEHTE

SEQ ID NO: 280 - protein, 31 aa, Unknown
  Present in Carya illinoinensis and Juglans regia
GAMMVPHYNSKATVVVYVVEGTGRFEMACPH

SEQ ID NO: 281 - protein, 19 aa, Prunus dulcis
KEKRQYQQCKQSCEKSSQH

SEQ ID NO: 282 - protein, 538 aa, Homo sapiens
SSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYY

SEQ ID NO: 283 - protein, 359 aa, Pisum sativum
VIAVSLTDIRSSNNQLDQMPRRFYLAGNHEQEFLQYQHQQGGKQEQENEGNNIFSGFKRDYLEDAFNVNRHIVDRLQGRNEDEEKGAIVKVKGGLSIISPPEKQARHQRGSRQEEDEDEEKQPRHQRGSRQEEEEDEDEERQPRHQRRRGEEEEEDKKERGGSQKGKSRRQGDNGLEETVCTAKLRLNIGPSSSPDIYNPEAGRIKTVTSLDLPVLRWLKLSAEHGSLHKNAMFVPHYNLNANSIIYALKGRARLQVVNCNGNTVFDGELEAGRALTVPQNYAVAAKSLSDRFSYVAFKTNDRAGIARLAGTSSVINNLPLDVVAATFNLQRNEARQLKSNNPFKFLVPARESENRASA

SEQ ID NO: 284 - protein, 338 aa, Cicer arietinum
VIAVSLIDTSSFQNQLDQMPRRFYLAGNHEQEFLRYQQEGSEEEENEGGNIFSGFKRDFLEDALNVNRRIVNKLQGRNEDEEKGAIVKVKGGLSITTPPEKEPRQKRGSRQEEDEDEDEKRQPHRHSRQDEDEDEKRQPHHHSRGGSKSQRDNGFEETICTARLHQNIGSSSSPDIYNPQAGRIKTVTSFDLQALRFLKLSAEFGSLHKNAMFVPHYNLNANSILYALKGRARLLYALNCKGNSVFDGELEAGRALIVPQNFAIAAKSLSDRFSYVAFKTNDRALINVCQKKLLQLLSIWKEMRPGSSSSTAPFHFLFHPAVTQTTKQQLDLVPNQYE

SEQ ID NO: 285 - protein, 392 aa, Homo sapiens
QFLQQCCSREMRIEEAKGFRAVQLKKTETYEKENCGGFRLIYPSLNSEKYEKFFQDNNSLFQNTVASRAREEYARQLIQELRLKREKKPFQMKKKVEMQGESAGEQVRKKGMRGWQQKQQQKDKAATQASKQYIQPLTLVSYTPDLLLSVRGERKNETDSSLNQEAPTEEASSVFPKLTSAKPFSSLPDLRNINLSSSKLEPSKPNFSIKEAKSASAVNVFTGTVHLTSVETTPESTTQLSISPKSPPTLAVTASSEYSGPETDRVVSFKCKKQQTPPHLTQKKMLKSFLPTKSKSFWESPNTNWTLLKSDMNKPHLISELLTKLQLSGKLSFFPAHYNPKLGMNNLSQNPSLPGECHSRSDSSGEKRQLDVSSLLLQSPQSYNVTLRDLLV

SEQ ID NO: 286 - protein, 101 aa, Homo sapiens
MSPLLRSICDITEIFNQYVSHDCDGAALTKKDLKNLLEREFGAVLRRPHDPKTVDLILELLDLDSNGRVDFNEFLLFIFKVAQACYYALGQATGLDEEKRA

SEQ ID NO: 287 - protein, 180 aa, Homo sapiens
ERDRKFLEEEQQLRRQERDRKFREQELRSQEPERKFLEEEQQLHRQQRQRKFLQEEQQLRRQERGQQRRQDRDRKFREEEQLRQEREEQQLSRQERDRKFRLEEQKVRRQEQERKFMEDEQQLRRQEGQQQLRQERDRKFREDEQLLQEREEQQLHRQERDRKFLEEEPQLRRQEREQQL

SEQ ID NO: 288 - protein, 223 aa, Homo sapiens
LERKFREEEQLRQETEQEQLRRQERYRKILEEEQLRPEREEQQLRRQERDRKFREEEQLRQEREEQQLRSQESDRKFREEEQLRQEREEQQLRPQQRDGKYRWEEEQLQLEEQEQRLRQERDRQYRAEEQFATQEKSRREEQELWQEEEQKRRQERERKLREEHIRRQQKEEQRHRQVGEIKSQEGKGHGRLLEPGTHQFASVPVRSSPLYEYIQEQRSQYRP

SEQ ID NO: 289 - protein, 339 aa, Arachis hypogaea
FPRRFNLAGNTEQEFLRYQQQSRQSRRRSLPYSPYSPQSQPRQEEREFSPRGQHSRRERAGQEEENEGGNIFSGFTPEFLEQAFQVDDRQIVQNLRGETESEEEGAIVTVRGGLRILSPDRKRRADEEEEYDEDEYEYDEEDRRRGRGSRGRGNGIEETICTASAKKNIGRNRSPDIYNPQAGSLKTANDLNLLILRWLGPSAEYGNLYRNALFVAHYNTNAHSIIYRLRGRAHVQVVDSNGNRVYDEELQEGHVLVVPQNFAVAGKSQSENFEYVAFKTDSRPSIANLAGENSVIDNLPEEVVANSYGLQREQARQLKNNNPFKFFVPPSQQSPRAVA

SEQ ID NO: 290 - protein, 510 aa, Homo sapiens
MLHQPTPGNRGLTARMEVGPATETFVLELQCLEDGGPGPDTLSGGSGGSESQEEEEPQERNSSPQRPAVSAPVGASEIAEETRPGQRELQLQQLEQQPEPQQQPQHEQLQQPQPHLELQQQPQQDGQQQLSQLQQEKHQSVHHQELKPELQLMHQQQQLQPQQVQEQQRLQQQQEQLQTQQAQEQQVLQQQEQLQQQVQEQQLLQQQQEQLQQQQLLQQQEQLQQQQFQQQQEQLQQQQQLLLLQQQGQLQQQLLQQQQAQLQQQLLEQQQAQLQQQLLLQQQEQLQQQQQQQLLQQQQEQLQQQQLQPPPLEPEEEEEVELELMPVDLGSEQELEQQRQELERQQELERQQEQRQLQLKLQEELQQLEQQLEQQQQQLEQQEVQLELTPVELGAQQQEVQLELTPVQPELQLELVPAAGGGGAAVPGAPAAVVVAPPGYVVVQELMVLPAVAAPAVVAIPGPAGSAALTPARQRRRRRARDRPTICGECGKGFSRSTDLVRHQATHTGE

SEQ ID NO: 291 - protein, 16 aa, Bos taurus
RFFVAPFPEVFGKEKV

SEQ ID NO: 292 - protein, 13 aa, Bos taurus
KHIQKEDVPSERY

SEQ ID NO: 293 - protein, 21 aa, Bos taurus
KHPIKHQGLPQEVLNENLLRF

SEQ ID NO: 294 - protein, 17 aa, Bos taurus
KHQGLPQEVLNENLLRF

SEQ ID NO: 295 - protein, 29 aa, Bos taurus
KEGIHAQQKEPMIGVNQELAYFYPELFRQ

SEQ ID NO: 296 - protein, 10 aa, Bos taurus
KEKVNELSKD

SEQ ID NO: 297 - protein, 14 aa, Bos taurus
RFFVAPFPEVFGKE

SEQ ID NO: 298 - protein, 15 aa, Bos taurus
RLHSMKEGIHAQQKE

SEQ ID NO: 299 - protein, 29 aa, Bos taurus
KEGIHAQQKEPMIGVNQELAYFYPELFRQ

SEQ ID NO: 300 - protein, 12 aa, Unknown
  Present in Bos taurus and Ovis aries
RYLGYLEQLLRL

SEQ ID NO: 301 - protein, 9 aa, Unknown
  Present in Bos taurus and Ovis aries
KEDVPSERY

SEQ ID NO: 302 - protein, 7 aa, Unknown
  Present in Bos taurus and Ovis aries
KTTMPLW

SEQ ID NO: 303 - protein, 7 aa, Unknown
  Present in Bos taurus and Ovis aries
KTTMPLW

SEQ ID NO: 304 - protein, 10 aa, Bos taurus
KFALPQYLKT

SEQ ID NO: 305 - protein, 13 aa, Bos taurus
RNAVPITPTLNRE

SEQ ID NO: 306 - protein, 11 aa, Bos taurus
KAMKPWIQPKT

SEQ ID NO: 307 - protein, 10 aa, Bos taurus
KTKVIPYVRY

SEQ ID NO: 308 - protein, 11 aa, Bos taurus
KAMKPWIQPKT

SEQ ID NO: 309 - protein, 11 aa, Bos taurus
KTKVIPYVRYL

SEQ ID NO: 310 - protein, 12 aa, Unknown
  Present in Bos taurus and Ovis aries
KITVDDKHYQKA

SEQ ID NO: 311 - protein, 13 aa, Unknown
  Present in Bos taurus and Ovis aries
KALNEINQFYQKF

SEQ ID NO: 312 - protein, 15 aa, Unknown
  Present in Bos taurus, Ovis aries and Sus scrofa
KLTEEEKNRLNFLKK

SEQ ID NO: 313 - protein, 9 aa, Bos taurus
KAVPYPQRD

SEQ ID NO: 314 - protein, 10 aa, Bos taurus
KVKEAMAPKH

SEQ ID NO: 315 - protein, 8 aa, Bos taurus
RGPFPIIV

SEQ ID NO: 316 - protein, 16 aa, Bos taurus
KVLPVPQKAVPYPQRD

SEQ ID NO: 317 - protein, 9 aa, Unknown
  Present in Bos taurus and Ovis aries
KVLPVPQKA

SEQ ID NO: 318 - protein, 10 aa, Unknown
  Present in Bos taurus and Ovis aries
KHKEMPFPKY

SEQ ID NO: 319 - protein, 10 aa, Unknown
  Present in Bos taurus and Ovis aries
KHKEMPFPKY

SEQ ID NO: 320 - protein, 20 aa, Bos taurus
RSPAQILQWQVLSNTVPAKS

SEQ ID NO: 321 - protein, 10 aa, Bos taurus
RFFSDKIAKY

SEQ ID NO: 322 - protein, 36 aa, Bos taurus
RYPSYGLNYYQQKPVALINNQFLPYPYYAKPAAVRS

SEQ ID NO: 323 - protein, 12 aa, Unknown
  Present in Bos taurus and Ovis aries
KYIPIQYVLSRY

SEQ ID NO: 324 - protein, 15 aa, Bos taurus
KAGLCQTFVYGGCRA

SEQ ID NO: 325 - protein, 13 aa, Bos taurus
RNNFKSAEDCMRT

SEQ ID NO: 326 - protein, 11 aa, Bos taurus
RIIRYFYNAKA

SEQ ID NO: 327 - protein, 16 aa, Bos taurus
RTPEVDDEALEKFDKA

SEQ ID NO: 328 - protein, 22 aa, Bos taurus
RVYVEELKPTPEGDLEILLQKW

SEQ ID NO: 329 - protein, 13 aa, Bos taurus
RTPEVDDEALEKF

SEQ ID NO: 330 - protein, 9 aa, Unknown
  Present in Bos taurus and Ovis aries
KALPMHIRL

SEQ ID NO: 331 - protein, 12 aa, Unknown
  Present in Bos taurus and Ovis aries
KVLVLDTDYKKY

SEQ ID NO: 332 - protein, 10 aa, Unknown
  Present in Bos taurus and Ovis aries
KTKIPAVFKI

SEQ ID NO: 333 - protein, 9 aa, Unknown
  Present in Bos taurus and Ovis aries
KALPMHIRL

SEQ ID NO: 334 - protein, 19 aa, Bos taurus
KLGSVYTEGGFVEGVNKKL

SEQ ID NO: 335 - protein, 26 aa, Bos taurus
KRAISQSGVGLCPWAIQQDPLFWAKR

SEQ ID NO: 336 - protein, 23 aa, Bos taurus
RCMLDRNEDMLITGGRHPFLARY

SEQ ID NO: 337 - protein, 22 aa, Bos taurus
RNQPEPTVEEIEDAFQGNLCRC

SEQ ID NO: 338 - protein, 23 aa, Bos taurus
RCMLDRNEDMLITGGRHPFLARY

SEQ ID NO: 339 - protein, 23 aa, Bos taurus
RCMLDRNEDMLITGGRHPFLARY

SEQ ID NO: 340 - protein, 26 aa, Bos taurus
KLGCGEGGCGACTVMLSKYDRLQDKI

SEQ ID NO: 341 - protein, 23 aa, Bos taurus
RVFVQKEILDQFTEEVVKQTQRI

SEQ ID NO: 342 - protein, 19 aa, Bos taurus
RVFVQKEILDQFTEEVVKQ

SEQ ID NO: 343 - protein, 14 aa, Bos taurus
KEILDQFTEEVVKQ

SEQ ID NO: 344 - protein, 23 aa, Bos taurus
RVIATFTCSGEKEVNLAVQDAKA

SEQ ID NO: 345 - protein, 10 aa, Bos taurus
KALGGEDVRV

SEQ ID NO: 346 - protein, 17 aa, Bos taurus
KTPIVGQPSIPGGPVRL

SEQ ID NO: 347 - protein, 12 aa, Unknown
  Present in Bos taurus and Ovis aries
KHTLNQIDSVKV

SEQ ID NO: 348 - protein, 15 aa, Unknown
  Present in Bos taurus and Ovis aries
RGYKHTLNQIDSVKV

SEQ ID NO: 349 - protein, 18 aa, Bos taurus
KSPPFFEDLTLDLQPPKS

SEQ ID NO: 350 - protein, 16 aa, Bos taurus
RADSLKKNQDLEFERN

SEQ ID NO: 351 - protein, 23 aa, Bos taurus
KTVDNFVALATGEKGFGYKDSKF

SEQ ID NO: 352 - protein, 10 aa, Bos taurus
KGFGYKDSKF

SEQ ID NO: 353 - protein, 13 aa, Bos taurus
RVYVVDVATEPRA

SEQ ID NO: 354 - protein, 15 aa, Bos taurus
RLVGQIFLGGSIVKG

SEQ ID NO: 355 - protein, 12 aa, Bos taurus
KLSISETYDLKS

SEQ ID NO: 356 - protein, 10 aa, Unknown
  Present in Bos taurus and Ovis aries
KAALTIDEKG

SEQ ID NO: 357 - protein, 17 aa, Unknown
  Present in Bos taurus and Ovis aries
KSVLGDVGITEVFSDRA

SEQ ID NO: 358 - protein, 12 aa, Bos taurus
KQIPLTCIVDKR

SEQ ID NO: 359 - protein, 11 aa, Bos taurus
KVTISCSGGRS

SEQ ID NO: 360 - protein, 14 aa, Bos taurus
RFLEDYFDGNLKRY

SEQ ID NO: 361 - protein, 14 aa, Bos taurus
RAIQAAFFYLEPRH

SEQ ID NO: 362 - protein, 29 aa, Bos taurus
KYVRPGGGFTPNFQLFEKGDVNGEKEQKF

SEQ ID NO: 363 - protein, 13 aa, Bos taurus
KYHALYINALQKL

SEQ ID NO: 364 - protein, 11 aa, Bos taurus
KTPTLEKQGKK

SEQ ID NO: 365 - protein, 10 aa, Bos taurus
KLAVPIILRV

SEQ ID NO: 366 - protein, 12 aa, Bos taurus
KVLSGSIEKAKQ

SEQ ID NO: 367 - protein, 10 aa, Bos taurus
RTVSISPTKK

SEQ ID NO: 368 - protein, 15 aa, Bos taurus
KLVEFPLVAAWYQRI

SEQ ID NO: 369 - protein, 11 aa, Bos taurus
KLQHFFIGNRK

SEQ ID NO: 370 - protein, 22 aa, Bos taurus
REVSNKIVGYLDEEGVLDQNRS

SEQ ID NO: 371 - protein, 9 aa, Bos taurus
RATDLVPRI

SEQ ID NO: 372 - protein, 9 aa, Bos taurus
RFEILPTRS

SEQ ID NO: 373 - protein, 12 aa, Unknown
  Present in Bos taurus and Ovis aries
RGSPAANVGVKV

SEQ ID NO: 374 - protein, 11 aa, Unknown
  Present in Bos taurus and Ovis aries
KSSELVSANRL

SEQ ID NO: 375 - protein, 19 aa, Unknown
  Present in Bos taurus and Rattus norvegicus
KLLSTLCSADVCQCAEGKC

SEQ ID NO: 376 - protein, 17 aa, Unknown
  Present in Bos taurus and Ovis aries
KECHLAQVPSHAVVARS

SEQ ID NO: 377 - protein, 15 aa, Oryza sativa
RWCAVAGGRLDSGKQ

SEQ ID NO: 378 - protein, 16 aa, Oryza sativa
KLFNIIEPDVAVFGKK

SEQ ID NO: 379 - protein, 14 aa, Unknown
  Present in Oryza sativa and Gloeobacter violaceus
KTIEVDNTDAEGRL

SEQ ID NO: 380 - protein, 11 aa, Unknown
  Present in various nonhuman organisms
RHPGWQGTLKA

SEQ ID NO: 381 - protein, 9 aa, Unknown
  Present in various nonhuman organisms
KGLTSLLRS

SEQ ID NO: 382 - protein, 13 aa, Unknown
  Present in various nonhuman organisms
KAMIAYWTNFART

SEQ ID NO: 383 - protein, 13 aa, Unknown
  Present in various nonhuman organisms
KAMIAYWTNFART

SEQ ID NO: 384 - protein, 18 aa, Unknown
  Present in various nonhuman organisms
KEVVLEEGTIAFKNWVKT

SEQ ID NO: 385 - protein, 10 aa, Unknown
  Present in various nonhuman organisms
RLGGAEIART

SEQ ID NO: 386 - protein, 11 aa, Unknown
  Present in various nonhuman organisms
KIFERCELART

SEQ ID NO: 387 - protein, 14 aa, Unknown
  Present in various nonhuman organisms
KFESNFNTQATNRN

SEQ ID NO: 388 - protein, 15 aa, Unknown
  Present in various nonhuman organisms
KKEVVLEEGTIAFKN

SEQ ID NO: 389 - protein, 10 aa, Unknown
  Present in various nonhuman organisms
RADLSGITKE

SEQ ID NO: 390 - protein, 9 aa, Unknown
  Present in various nonhuman organisms
RIEYDPNRS

SEQ ID NO: 391 - protein, 13 aa, Unknown
  Present in various nonhuman organisms
RLDSPATPERIRN

SEQ ID NO: 392 - protein, 18 aa, Unknown
  Present in various nonhuman organisms
RNTDGSTDYGILQINSRW

SEQ ID NO: 393 - protein, 20 aa, Unknown
  Present in various nonhuman organisms
RSALFAQINQGESITHALKH

SEQ ID NO: 394 - protein, 11 aa, Unknown
  Present in various nonhuman organisms
RYEVPLETPRV

SEQ ID NO: 395 - protein, 23 aa, Bos taurus
RVFVQKEILDQFTEEVVKQTQRI

SEQ ID NO: 396 - protein, 19 aa, Bos taurus
RVFVQKEILDQFTEEVVKQ

SEQ ID NO: 397 - protein, 14 aa, Bos taurus
KEILDQFTEEVVKQ

SEQ ID NO: 398 - protein, 23 aa, Bos taurus
RVIATFTCSGEKEVNLAVQDAKA

SEQ ID NO: 399 - protein, 14 aa, Bos taurus
RAIQAAFFYLEPRH

SEQ ID NO: 400 - protein, 12 aa, Bos taurus
KLSISETYDLKS

SEQ ID NO: 401 - protein, 10 aa, Unknown
  Present in Bos taurus and Ovis aries
KAALTIDEKG

SEQ ID NO: 402 - protein, 17 aa, Unknown
  Present in Bos taurus and Ovis aries
KSVLGDVGITEVFSDRA

SEQ ID NO: 403 - protein, 10 aa, Bos taurus
KALGGEDVRV

SEQ ID NO: 404 - protein, 17 aa, Bos taurus
KTPIVGQPSIPGGPVRL

SEQ ID NO: 405 - protein, 12 aa, Unknown
  Present in Bos taurus and Ovis aries
KHTLNQIDSVKV

SEQ ID NO: 406 - protein, 15 aa, Unknown
  Present in Bos taurus and Ovis aries
RGYKHTLNQIDSVKV

SEQ ID NO: 407 - protein, 24 aa, Bos taurus
AQFVPLPVSVSVEFAVAATDCIAK

SEQ ID NO: 408 - protein, 10 aa, Bos taurus
KLAVPIILRV

SEQ ID NO: 409 - protein, 9 aa, Bos taurus
GGVSLPEWV

SEQ ID NO: 410 - protein, 9 aa, Bos taurus
LDQWLCEKL

SEQ ID NO: 411 - protein, 13 aa, Bos taurus
NICNISCDKFLDD

SEQ ID NO: 412 - protein, 9 aa, Bos taurus
ENLLRFFVA

SEQ ID NO: 413 - protein, 8 aa, Bos taurus
EKVNELSK

SEQ ID NO: 414 - protein, 12 aa, Bos taurus
FFVAPFPEVFGK

SEQ ID NO: 415 - protein, 11 aa, Bos taurus
HIQKEDVPSER

SEQ ID NO: 416 - protein, 15 aa, Bos taurus
HQGLPQEVLNENLLR

SEQ ID NO: 417 - protein, 10 aa, Bos taurus
YLGYLEQLLR

SEQ ID NO: 418 - protein, 16 aa, Bos taurus
RFFVAPFPEVFGKEKV

SEQ ID NO: 419 - protein, 13 aa, Bos taurus
KHIQKEDVPSERY

SEQ ID NO: 420 - protein, 21 aa, Bos taurus
KHPIKHQGLPQEVLNENLLRF

SEQ ID NO: 421 - protein, 17 aa, Bos taurus
KHQGLPQEVLNENLLRF

SEQ ID NO: 422 - protein, 29 aa, Bos taurus
KEGIHAQQKEPMIGVNQELAYFYPELFRQ

SEQ ID NO: 423 - protein, 10 aa, Bos taurus
KEKVNELSKD

SEQ ID NO: 424 - protein, 14 aa, Bos taurus
RFFVAPFPEVFGKE

SEQ ID NO: 425 - protein, 15 aa, Bos taurus
RLHSMKEGIHAQQKE

SEQ ID NO: 426 - protein, 29 aa, Bos taurus
KEGIHAQQKEPMIGVNQELAYFYPELFRQ

SEQ ID NO: 427 - protein, 12 aa, Unknown
  Present in Bos taurus and Ovis aries
RYLGYLEQLLRL

SEQ ID NO: 428 - protein, 9 aa, Unknown
  Present in Bos taurus and Ovis aries
KEDVPSERY

SEQ ID NO: 429 - protein, 7 aa, Unknown
  Present in Bos taurus and Ovis aries
KTTMPLW

SEQ ID NO: 430 - protein, 7 aa, Unknown
  Present in Bos taurus and Ovis aries
KTTMPLW

SEQ ID NO: 431 - protein, 10 aa, Bos taurus
KFALPQYLKT

SEQ ID NO: 432 - protein, 13 aa, Bos taurus
RNAVPITPTLNRE

SEQ ID NO: 433 - protein, 11 aa, Bos taurus
KAMKPWIQPKT

SEQ ID NO: 434 - protein, 10 aa, Bos taurus
KTKVIPYVRY

SEQ ID NO: 435 - protein, 11 aa, Bos taurus
KAMKPWIQPKT

SEQ ID NO: 436 - protein, 11 aa, Bos taurus
KTKVIPYVRYL

SEQ ID NO: 437 - protein, 12 aa, Unknown
  Present in Bos taurus and Ovis aries
KITVDDKHYQKA

SEQ ID NO: 438 - protein, 13 aa, Unknown
  Present in Bos taurus and Ovis aries
KALNEINQFYQKF

SEQ ID NO: 439 - protein, 15 aa, Unknown
  Present in Bos taurus, Ovis aries and Sus scrofa
KLTEEEKNRLNFLKK

SEQ ID NO: 440 - protein, 11 aa, Unknown
  Present in Bos taurus and Ovis aries
KSSELVSANRL

SEQ ID NO: 441 - protein, 10 aa, Bos taurus
HEQGMDQDKN

SEQ ID NO: 442 - protein, 14 aa, Bos taurus
SSLSDIDQENNNNK

SEQ ID NO: 443 - protein, 18 aa, Bos taurus
TLQIAEIKDNSGPRSNED

SEQ ID NO: 444 - protein, 20 aa, Bos taurus
VYVEQLKPTPEGDLEILLQK

SEQ ID NO: 445 - protein, 11 aa, Bos taurus
DLKLVEQQNPK

SEQ ID NO: 446 - protein, 10 aa, Bos taurus
LPQNIPPLTQ

SEQ ID NO: 447 - protein, 8 aa, Bos taurus
QPLPPTVM

SEQ ID NO: 448 - protein, 12 aa, Bos taurus
PVVVPPFLQPEV

SEQ ID NO: 449 - protein, 9 aa, Bos taurus
KAVPYPQRD

SEQ ID NO: 450 - protein, 10 aa, Bos taurus
KVKEAMAPKH

SEQ ID NO: 451 - protein, 8 aa, Bos taurus
RGPFPIIV

SEQ ID NO: 452 - protein, 16 aa, Bos taurus
KVLPVPQKAVPYPQRD

SEQ ID NO: 453 - protein, 9 aa, Unknown
  Present in Bos taurus and Ovis aries
KVLPVPQKA

SEQ ID NO: 454 - protein, 10 aa, Unknown
  Present in Bos taurus and Ovis aries
KHKEMPFPKY

SEQ ID NO: 455 - protein, 10 aa, Unknown
  Present in Bos taurus and Ovis aries
KHKEMPFPKY

SEQ ID NO: 456 - protein, 7 aa, Bos taurus
VALNKLK

SEQ ID NO: 457 - protein, 7 aa, Bos taurus
ALPMHIR

SEQ ID NO: 458 - protein, 8 aa, Bos taurus
IDALNENK

SEQ ID NO: 459 - protein, 8 aa, Bos taurus
LIVTQTMK

SEQ ID NO: 460 - protein, 14 aa, Bos taurus
LSFNPTQLEEQCHI

SEQ ID NO: 461 - protein, 8 aa, Bos taurus
TKIPAVFK

SEQ ID NO: 462 - protein, 11 aa, Bos taurus
TPEVDDEALEK

SEQ ID NO: 463 - protein, 14 aa, Bos taurus
TPEVDDEALEKFDK

SEQ ID NO: 464 - protein, 10 aa, Bos taurus
VLVLDTDYKK

SEQ ID NO: 465 - protein, 20 aa, Bos taurus
VYVEELKPTPEGDLEILLQK

SEQ ID NO: 466 - protein, 9 aa, Bos taurus
WENDECAQK

SEQ ID NO: 467 - protein, 10 aa, Bos taurus
WENDECAQKK

SEQ ID NO: 468 - protein, 20 aa, Bos taurus
SLAMAASDISLLDAQSAPLR

SEQ ID NO: 469 - protein, 16 aa, Bos taurus
RTPEVDDEALEKFDKA

SEQ ID NO: 470 - protein, 22 aa, Bos taurus
RVYVEELKPTPEGDLEILLQKW

SEQ ID NO: 471 - protein, 13 aa, Bos taurus
RTPEVDDEALEKF

SEQ ID NO: 472 - protein, 9 aa, Unknown
  Present in Bos taurus and Ovis aries
KALPMHIRL

SEQ ID NO: 473 - protein, 12 aa, Unknown
  Present in Bos taurus and Ovis aries
KVLVLDTDYKKY

SEQ ID NO: 474 - protein, 10 aa, Unknown
  Present in Bos taurus and Ovis aries
KTKIPAVFKI

SEQ ID NO: 475 - protein, 9 aa, Unknown
  Present in Bos taurus and Ovis aries
KALPMHIRL

SEQ ID NO: 476 - protein, 19 aa, Bos taurus
KLGSVYTEGGFVEGVNKKL

SEQ ID NO: 477 - protein, 26 aa, Bos taurus
KRAISQSGVGLCPWAIQQDPLFWAKR

SEQ ID NO: 478 - protein, 14 aa, Bos taurus
NVTRQAYWQIHMDQ

SEQ ID NO: 479 - protein, 11 aa, Bos taurus
NIIKSGSDEVQ

SEQ ID NO: 480 - protein, 19 aa, Unknown
  Present in Bos taurus and Rattus norvegicus
KLLSTLCSADVCQCAEGKC

SEQ ID NO: 481 - protein, 11 aa, Bos taurus
KVTISCSGGRS

SEQ ID NO: 482 - protein, 18 aa, Bos taurus
KSPPFFEDLTLDLQPPKS

SEQ ID NO: 483 - protein, 16 aa, Bos taurus
RADSLKKNQDLEFERN

SEQ ID NO: 484 - protein, 9 aa, Bos taurus
EKESLGWQK

SEQ ID NO: 485 - protein, 13 aa, Bos taurus
KYHALYINALQKL

SEQ ID NO: 486 - protein, 12 aa, Bos taurus
KVLSGSIEKAKQ

SEQ ID NO: 487 - protein, 15 aa, Bos taurus
VNLLVDRQWQAVRNR

SEQ ID NO: 488 - protein, 15 aa, Bos taurus
QNLAFVSMLNDIAAP

SEQ ID NO: 489 - protein, 10 aa, Bos taurus
RTVSISPTKK

SEQ ID NO: 490 - protein, 29 aa, Bos taurus
KYVRPGGGFTPNFQLFEKGDVNGEKEQKF

SEQ ID NO: 491 - protein, 15 aa, Bos taurus
KLVEFPLVAAWYQRI

SEQ ID NO: 492 - protein, 14 aa, Bos taurus
NGEGQVLFETEISR

SEQ ID NO: 493 - protein, 14 aa, Bos taurus
VSITCSGSSSNIGR

SEQ ID NO: 494 - protein, 11 aa, Bos taurus
KLQHFFIGNRK

SEQ ID NO: 495 - protein, 14 aa, Bos taurus
IKVMNDLSPKSNLR

SEQ ID NO: 496 - protein, 9 aa, Bos taurus
ESTVATLED

SEQ ID NO: 497 - protein, 7 aa, Bos taurus
IESPPEI

SEQ ID NO: 498 - protein, 11 aa, Bos taurus
SCQAQPTTMAR

SEQ ID NO: 499 - protein, 20 aa, Bos taurus
RSPAQILQWQVLSNTVPAKS

SEQ ID NO: 500 - protein, 10 aa, Bos taurus
RFFSDKIAKY

SEQ ID NO: 501 - protein, 36 aa, Bos taurus; identical to SEQ ID NO: 322

SEQ ID NO: 502 - protein, 12 aa, Unknown
  Present in Bos taurus and Ovis aries
KYIPIQYVLSRY

SEQ ID NO: 503 - protein, 22 aa, Bos taurus
REVSNKIVGYLDEEGVLDQNRS

SEQ ID NO: 504 - protein, 13 aa, Bos taurus
NLLFNDNTECLAK

SEQ ID NO: 505 - protein, 17 aa, Unknown
  Present in Bos taurus and Ovis aries
KECHLAQVPSHAVVARS

SEQ ID NO: 506 - protein, 8 aa, Bos taurus
ESPQTHYY

SEQ ID NO: 507 - protein, 10 aa, Bos taurus
GSNFQLDQLQ

SEQ ID NO: 508 - protein, 9 aa, Bos taurus
VAVVKKGSN

SEQ ID NO: 509 - protein, 8 aa, Bos taurus
YEELGTEY

SEQ ID NO: 510 - protein, 23 aa, Bos taurus
EKQLPNGDWPQENISGVFNKSCA

SEQ ID NO: 511 - protein, 11 aa, Bos taurus
KLLNNITNDLR

SEQ ID NO: 512 - protein, 12 aa, Bos taurus
KQIPLTCIVDKR

SEQ ID NO: 513 - protein, 10 aa, Bos taurus
QMERALLENE

SEQ ID NO: 514 - protein, 11 aa, Bos taurus
IQQNSSTTEKI

SEQ ID NO: 515 - protein, 10 aa, Bos taurus
KFNITDTLMQ

SEQ ID NO: 516 - protein, 16 aa, Bos taurus
EHLYQENQYLEQENTQ

SEQ ID NO: 517 - protein, 20 aa, Bos taurus
QEELENRTSETNTPQGNQEY

SEQ ID NO: 518 - protein, 14 aa, Bos taurus
NKHSNLIESQENSK

SEQ ID NO: 519 - protein, 15 aa, Bos taurus
KAGLCQTFVYGGCRA

SEQ ID NO: 520 - protein, 13 aa, Bos taurus
RNNFKSAEDCMRT

SEQ ID NO: 521 - protein, 11 aa, Bos taurus
RIIRYFYNAKA

SEQ ID NO: 522 - protein, 23 aa, Bos taurus
KTVDNFVALATGEKGFGYKDSKF

SEQ ID NO: 523 - protein, 10 aa, Bos taurus
KGFGYKDSKF

SEQ ID NO: 524 - protein, 9 aa, Bos taurus
RATDLVPRI

SEQ ID NO: 525 - protein, 14 aa, Bos taurus
RFLEDYFDGNLKRY

SEQ ID NO: 526 - protein, 9 aa, Bos taurus
RFEILPTRS

SEQ ID NO: 527 - protein, 13 aa, Bos taurus
RVYVVDVATEPRA

SEQ ID NO: 528 - protein, 15 aa, Bos taurus
RLVGQIFLGGSIVKG

SEQ ID NO: 529 - protein, 10 aa, Bos taurus
CASFRENVLR

SEQ ID NO: 530 - protein, 8 aa, Bos taurus
AEFVEVTK

SEQ ID NO: 531 - protein, 13 aa, Bos taurus
DAFLGSFLYEYSR

SEQ ID NO: 532 - protein, 8 aa, Bos taurus
DLGEEHFK

SEQ ID NO: 533 - protein, 10 aa, Bos taurus
DTHKSEIAHR

SEQ ID NO: 534 - protein, 10 aa, Bos taurus
DVCKNYQEAK

SEQ ID NO: 535 - protein, 10 aa, Bos taurus
FKDLGEEHFK

SEQ ID NO: 536 - protein, 11 aa, Bos taurus
HLVDEPQNLIK

SEQ ID NO: 537 - protein, 10 aa, Bos taurus
LVNELTEFAK

SEQ ID NO: 538 - protein, 8 aa, Bos taurus
QNCDQFEK

SEQ ID NO: 539 - protein, 12 aa, Bos taurus
RHPEYAVSVLLR

SEQ ID NO: 540 - protein, 12 aa, Bos taurus
SLHTLFGDELCK

SEQ ID NO: 541 - protein, 13 aa, Bos taurus
TCVADESHAGCEK

SEQ ID NO: 542 - protein, 9 aa, Bos taurus
GKYLYEIAR

SEQ ID NO: 543 - protein, 10 aa, Bos taurus
KQTALVELLK

SEQ ID NO: 544 - protein, 11 aa, Bos taurus
KTPTLEKQGKK

SEQ ID NO: 545 - protein, 12 aa, Unknown
  Present in Bos taurus and Ovis aries
RGSPAANVGVKV

SEQ ID NO: 546 - protein, 8 aa, Bos taurus
HHIELRWK

SEQ ID NO: 547 - protein, 15 aa, Bos taurus
QKYGVVKENVIDLTK

SEQ ID NO: 548 - protein, 23 aa, Bos taurus
RCMLDRNEDMLITGGRHPFLARY

SEQ ID NO: 549 - protein, 22 aa, Bos taurus
RNQPEPTVEEIEDAFQGNLCRC

SEQ ID NO: 550 - protein, 23 aa, Bos taurus
RCMLDRNEDMLITGGRHPFLARY

SEQ ID NO: 551 - protein, 23 aa, Bos taurus
RCMLDRNEDMLITGGRHPFLARY

SEQ ID NO: 552 - protein, 26 aa, Bos taurus
KLGCGEGGCGACTVMLSKYDRLQDKI

SEQ ID NO: 553 - protein, 12 aa, Bos taurus
NGNNPNCCMNQK

SEQ ID NO: 554 - protein, 12 aa, Zea mays
QFKPEEMTNIIK

SEQ ID NO: 555 - protein, 12 aa, Triticum aestivum
QQQTLQQILQQQ

SEQ ID NO: 556 - protein, 13 aa, Triticum aestivum
QVLQQSSYQQLQQ

SEQ ID NO: 557 - protein, 8 aa, Unknown
  Present in Arachis hypogaea and Arachis duranensis
RPSHQQPR

SEQ ID NO: 558 - protein, 10 aa, Arachis hypogaea
QQWELQGDRR

SEQ ID NO: 559 - protein, 13 aa, Arachis hypogaea
ANLRPCEQHLMQK

SEQ ID NO: 560 - protein, 29 aa, Arachis hypogaea
CCNELNEFENNQRCMCEALQQIMENQSDR

SEQ ID NO: 561 - protein, 9 aa, Arachis hypogaea
NLPQQCGLR

SEQ ID NO: 562 - protein, 10 aa, Arachis hypogaea
CDLEVESGGR

SEQ ID NO: 563 - protein, 15 aa, Oryza sativa
RWCAVAGGRLDSGKQ

SEQ ID NO: 564 - protein, 16 aa, Oryza sativa
KLFNIIEPDVAVFGKK

SEQ ID NO: 565 - protein, 14 aa, Unknown
  Present in Oryza sativa and Gloeobacter violaceus
KTIEVDNTDAEGRL

SEQ ID NO: 566 - protein, 11 aa, Unknown
  Present in various nonhuman organisms
RHPGWQGTLKA

SEQ ID NO: 567 - protein, 9 aa, Unknown
  Present in various nonhuman organisms
KGLTSLLRS

SEQ ID NO: 568 - protein, 13 aa, Unknown
  Present in various nonhuman organisms
KAMIAYWTNFART

SEQ ID NO: 569 - protein, 13 aa, Unknown
  Present in various nonhuman organisms
KAMIAYWTNFART

SEQ ID NO: 570 - protein, 18 aa, Unknown
  Present in various nonhuman organisms
KEVVLEEGTIAFKNWVKT

SEQ ID NO: 571 - protein, 10 aa, Unknown
  Present in various nonhuman organisms
RLGGAEIART

SEQ ID NO: 572 - protein, 11 aa, Unknown
  Present in various nonhuman organisms
KIFERCELART

SEQ ID NO: 573 - protein, 14 aa, Unknown
  Present in various nonhuman organisms
KFESNFNTQATNRN

SEQ ID NO: 574 - protein, 15 aa, Unknown
  Present in various nonhuman organisms
KKEVVLEEGTIAFKN

SEQ ID NO: 575 - protein, 10 aa, Unknown
  Present in various nonhuman organisms
RADLSGITKE

SEQ ID NO: 576 - protein, 9 aa, Unknown
  Present in various nonhuman organisms
RIEYDPNRS

SEQ ID NO: 577 - protein, 13 aa, Unknown
  Present in various nonhuman organisms
RLDSPATPERIRN

SEQ ID NO: 578 - protein, 18 aa, Unknown
  Present in various nonhuman organisms
RNTDGSTDYGILQINSRW

SEQ ID NO: 579 - protein, 20 aa, Unknown
  Present in various nonhuman organisms
RSALFAQINQGESITHALKH

SEQ ID NO: 580 - protein, 11 aa, Unknown
  Present in various nonhuman organisms
RYEVPLETPRV

SEQ ID NO: 581 - protein, 7 aa, Unknown
  Present in various nonhuman organisms
EQVQELR

SEQ ID NO: 582 - protein, 10 aa, Unknown
  Present in various nonhuman organisms
MQDQLDQVQK

SEQ ID NO: 583 - protein, 13 aa, Unknown
  Present in various nonhuman organisms
KELKKVEADGEND

SEQ ID NO: 584 - protein, 11 aa, Unknown
  Present in various nonhuman organisms
QIANSDEVEKI

SEQ ID NO: 585 - protein, 13 aa, Unknown
  Present in various nonhuman organisms
KCAADESAENCDK

SEQ ID NO: 586 - protein, 13 aa, Unknown
  Present in various nonhuman organisms
KEPERNECFLQHK

SEQ ID NO: 587 - protein, 15 aa, Unknown
  Present in various nonhuman organisms
PCFSALQVDETYVPK

SEQ ID NO: 588 - protein, 12 aa, Unknown
  Present in various nonhuman organisms
YICENQDSISTK

SEQ ID NO: 589 - protein, 12 aa, Unknown
  Present in various nonhuman organisms
SALQVDETYVPK

SEQ ID NO: 590 - protein, 14 aa, Unknown
  Present in various nonhuman organisms
KEQVARFTAGTNPK